(19) United States
(12) Patent Application Publication
(10) Pub. No.: US 2024/0252640 A1
(43) Pub. Date: Aug. 1, 2024

(54) ANTIGEN RECOGNIZING RECEPTORS TARGETING DLL3 AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Janneke E. Jaspers, New York, NY (US); Marjan ZAMAN, New York, NY (US); Renier J. BRENTJENS, Short Hills, NJ (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 18/591,403

(22) Filed: Feb. 29, 2024

Related U.S. Application Data
(63) Continuation of application No. PCT/US2022/042429, filed on Sep. 2, 2022.
(60) Provisional application No. 63/240,189, filed on Sep. 2, 2021.

(51) Int. Cl. A61K 39/00; A61P 35/00; C07K 14/705; C07K 14/725; C07K 16/18; C12N 5/0783

(52) U.S. Cl. CPC A61K 39/4631; A61K 39/4611; A61K 39/4643; A61P 35/00; C07K 14/7051; C07K 14/70521; C07K 16/18; C12N 5/0636; A61K 2039/572; C07K 2317/622; C07K 2319/02; C07K 2319/03; C12N 2510/00

(57) ABSTRACT

The presently disclosed subject matter provides for antigen-recognizing receptors that specifically target DLL3 and cells comprising such DLL3-targeted antigen-recognizing receptors. The presently disclosed subject matter further provides uses of the DLL3-targeted antigen-recognizing receptors for treatment.

### CROSS REFERENCE TO RELATED APPLICATIONS

This application is a continuation of International Application No. PCT/US22/042429 filed on Sep. 2, 2022, which claims priority to U.S. Provisional Patent Application No. 63/240,189, filed on Sep. 2, 2021, the contents of which are incorporated by reference in their entireties, and to each of which priority is claimed.

### SEQUENCE LISTING

The present application contains a Sequence Listing which has been submitted via EFS-Web and is hereby incorporated by reference in its entirety. Said Sequence Listing, created on Feb. 26, 2024, is named 0727341566.xml and is 264,849 bytes in size.

### 1. INTRODUCTION

The presently disclosed subject matter provides methods and compositions for immunotherapies. It relates to antigen-recognizing receptors (e.g., chimeric antigen receptors (CARs)) that specifically target DLL3, cells comprising such receptors, and methods of using such cells for treatments.

### 2. BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed chimeric antigen receptors (CARs), specific to selected antigens. Targeted T cell therapy using CARs has shown recent clinical success in treating hematologic malignancies and solid tumors.

DLL3 is selectively expressed in high grade pulmonary neuroendocrine tumors of the lung (LU-NETs) and other neuroendocrine cancers. Lu-NETs embrace a heterogeneous family of neoplasms classified into four histological variants, namely typical carcinoid (TC), atypical carcinoid (AC), large cell neuroendocrine carcinoma (LCNEC) and small cell lung carcinoma (SCLC). Increased expression of DLL3 was observed in SCLC and LCNEC patient-derived xenograft tumors and was also confirmed in primary tumors. See Saunders et al., Sci Translational Medicine (302): 302ra136 (2015). Both SCLC and pulmonary LCNEC are high-grade and poor-prognosis tumors, with higher incidence in smokers. Pulmonary LCNEC exhibits biologically aggressive behavior, similarly to SCLC. Stage by stage, survival curves of pulmonary LCNEC and SCLC overlap, and in addition, survival is lower than other NSCLCs. Prognosis is poor even in patients with potentially resectable stage I lung cancer with 5-year survival rates ranging from 27% to 67%. See Iyoda A. et al., J Thorac Cardiovasc Surg. 138:446-453 (2009).

Given the significant role for DLL3 in various diseases or disorders, immunotherapies (e.g., CARs) targeting DLL3, are desired.

### 3. SUMMARY OF THE INVENTION

The presently disclosed subject matter provides antigen-recognizing receptors that specifically target DLL3 and cells comprising such DLL3-targeted antigen-recognizing receptors. The presently disclosed subject matter further provides uses of the DLL3-targeted antigen-recognizing receptors for treatment.

The presently disclosed subject matter provides an antigen-recognizing receptor, comprising an extracellular antigen-binding domain, a transmembrane domain, and an intracellular signaling domain, wherein the extracellular antigen-binding domain specifically binds to DLL3. In certain embodiments, the extracellular antigen-binding domain is a single-chain variable fragment (scFv). In certain embodiments, the extracellular antigen-binding domain is a human scFv. In certain embodiments, the extracellular antigen-binding domain is a Fab, which is optionally crosslinked. In certain embodiments, the extracellular antigen-binding domain is a F(ab)₂. In certain embodiments, one or more of the scFv, Fab and F(ab)₂ are comprised in a fusion protein with a heterologous sequence to form the extracellular antigen-binding domain.

In certain embodiments, the extracellular antigen-binding domain comprises:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof;
(b) a heavy chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof;
(c) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof;
(d) a heavy chain variable region comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof;
(e) a heavy chain variable region comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof;
(f) a heavy chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof;
(g) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof;
(h) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 64 or a conservative modification thereof;
(i) a heavy chain variable region comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof;
(j) a heavy chain variable region comprising a CDR1 comprising the amino acid sequence set forth in SEQ ID NO: 21 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof;
(k) a heavy chain variable region comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof;
(l) a heavy chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof;
(m) a heavy chain variable region comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof;
(n) a heavy chain variable region comprising a CDR1 comprising the amino acid sequence set forth in SEQ ID NO: 95 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof;
(o) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof;
(p) a heavy chain variable region comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof;
(q) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof;
(r) a heavy chain variable region comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof;
(s) a heavy chain variable region comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof;
(t) a heavy chain variable region comprising a CDR1 comprising the amino acid sequence set forth in SEQ ID NO: 96 or a conservative modification thereof, a CDR2 comprising the amino acid sequence set forth in SEQ ID NO: 167 or a conservative modification thereof, and a CDR3 comprising the amino acid sequence set forth in SEQ ID NO: 168 or a conservative modification thereof;
(u) a heavy chain variable region comprising a CDR1 comprising the amino acid sequence set forth in SEQ ID NO: 21 or a conservative modification thereof, a CDR2 comprising the amino acid sequence set forth in SEQ ID NO: 176 or a conservative modification thereof, and a CDR3 comprising the amino acid sequence set forth in SEQ ID NO: 177 or a conservative modification thereof;
(v) a heavy chain variable region comprising a CDR1 comprising the amino acid sequence set forth in SEQ ID NO: 184 or a conservative modification thereof, a CDR2 comprising the amino acid sequence set forth in SEQ ID NO: 185 or a conservative modification thereof, and a CDR3 comprising the amino acid sequence set forth in SEQ ID NO: 186 or a conservative modification thereof;
(w) a heavy chain variable region comprising a CDR1 comprising the amino acid sequence set forth in SEQ ID NO: 11 or a conservative modification thereof, a CDR2 comprising the amino acid sequence set forth in SEQ ID NO: 194 or a conservative modification thereof, and a CDR3 comprising the amino acid sequence set forth in SEQ ID NO: 195 or a conservative modification thereof; or
(x) a heavy chain variable region comprising a CDR1 comprising the amino acid sequence set forth in SEQ ID NO: 201 or a conservative modification thereof, a CDR2 comprising the amino acid sequence set forth in SEQ ID NO: 202 or a conservative modification thereof, and a CDR3 comprising the amino acid sequence set forth in SEQ ID NO: 203 or a conservative modification thereof.

In certain embodiments, the extracellular antigen-binding domain comprises: a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof.

In certain embodiments, the extracellular antigen-binding domain comprises: a heavy chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof.

In certain embodiments, the extracellular antigen-binding domain comprises: a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof.

In certain embodiments, the extracellular antigen-binding domain comprises:
(a) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof;
(b) a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising SEQ ID NO: 16 or a conservative modification thereof;
(c) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof;
(d) a light chain variable region comprising a CDR1 comprising the amino acid sequence set forth in SEQ ID NO: 31 or a conservative modification thereof, a CDR2 comprising SEQ ID NO: 32 or a conservative modification thereof, and a CDR3 comprising the amino acid sequence set forth in SEQ ID NO: 33 or a conservative modification thereof;
(e) a light chain variable region comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof and a CDR3 comprising the amino acid sequence set forth in SEQ ID NO: 41 or a conservative modification thereof;
(f) a light chain variable region comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof;
(g) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(h) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof;
(i) a light chain variable region comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof;
(j) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof;
(k) a light chain variable region comprising a CDR1 comprising the amino acid sequence set forth in SEQ ID NO: 90 or a conservative modification thereof, a CDR2 comprising the amino acid sequence set forth in SEQ ID NO: 280 or a conservative modification thereof, and a CDR3 comprising the amino acid sequence set forth in SEQ ID NO: 91 or a conservative modification thereof;
(l) a light chain variable region comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof;
(m) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification thereof;
(n) a light chain variable region comprising a CDR1 comprising the amino acid sequence set forth in SEQ ID NO: 117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification thereof;
(o) a light chain variable region comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification thereof;
(p) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification thereof;
(q) a light chain variable region comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification thereof;
(r) a light chain variable region comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification thereof;
(s) a light chain variable region comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(t) a light chain variable region comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification thereof;
(u) a light chain variable region comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification thereof;
(v) a light chain variable region comprising a CDR1 comprising the amino acid sequence set forth in SEQ ID NO: 178 or a conservative modification thereof, a CDR2 comprising the amino acid sequence set forth in SEQ ID NO: 50 or a conservative modification thereof, and a CDR3 comprising the amino acid sequence set forth in SEQ ID NO: 179 or a conservative modification thereof;
(w) a light chain variable region comprising a CDR1 comprising the amino acid sequence set forth in SEQ ID NO: 187 or a conservative modification thereof, a CDR2 comprising the amino acid sequence set forth in SEQ ID NO: 188 or a conservative modification thereof, and a CDR3 comprising the amino acid sequence set forth in SEQ ID NO: 189 or a conservative modification thereof;
(x) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 196 or a conservative modification thereof; or
(y) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 204 or a conservative modification thereof.

In certain embodiments, the extracellular antigen-binding domain comprises: a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof.

In certain embodiments, the extracellular antigen-binding domain comprises: a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising SEQ ID NO: 16 or a conservative modification thereof;

In certain embodiments, the extracellular antigen-binding domain comprises: a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof.

In certain embodiments, the extracellular antigen-binding domain comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
(c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
(d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
(f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
(h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
(i) a heavy chain variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
(j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 280, and a CDR3 comprising the amino acid sequence set forth in SEQ ID NO: 91;
(l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
(m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
(o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
(p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
(q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
(r) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
(s) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
(t) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(u) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
(v) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
(w) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171;
(x) a heavy chain variable region comprising a CDR1 comprising the amino acid sequence set forth in SEQ ID NO: 21, a CDR2 comprising the amino acid sequence set forth in SEQ ID NO: 176 and a CDR3 comprising the amino acid sequence set forth in SEQ ID NO: 177; and a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 79;
(y) a heavy chain variable region comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186; and a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189;
(z) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196; or
(aa) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

In certain embodiments, the extracellular antigen-binding domain comprises: a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the extracellular antigen-binding domain comprises: a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the extracellular antigen-binding domain comprises: a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23.

In certain embodiments, the extracellular antigen-binding domain comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205. In certain embodiments, the extracellular antigen-binding domain comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205. In certain embodiments, the extracellular antigen-binding domain comprises a heavy chain variable region comprising the amino acid sequence set forth in SEQ ID NO: 7. In certain embodiments, the extracellular antigen-binding domain comprises a heavy chain variable region comprising the amino acid sequence set forth in SEQ ID NO: 17. In certain embodiments, the extracellular antigen-binding domain comprises a heavy chain variable region comprising the amino acid sequence set forth in SEQ ID NO: 24.

In certain embodiments, the extracellular antigen-binding domain comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206. In certain embodiments, the extracellular antigen-binding domain comprises a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206. In certain embodiments, the extracellular antigen-binding domain comprises a light chain variable region comprising the amino acid sequence set forth in SEQ ID NO: 8. In certain embodiments, the extracellular antigen-binding domain comprises a light chain variable region comprising the amino acid sequence set forth in SEQ ID NO: 18. In certain embodiments, the extracellular antigen-binding domain comprises a light chain variable region comprising the amino acid sequence set forth in SEQ ID NO: 25.

In certain embodiments, the extracellular antigen-binding domain comprises: (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

In certain embodiments, the extracellular antigen-binding domain comprises: (a) a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and (b) a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206. In certain embodiments, the extracellular antigen-binding domain comprises: (a) a heavy chain variable region comprising the amino acid sequence set forth in SEQ ID NO: 7, SEQ ID NO: 17, or SEQ ID NO: 24; and (b) a light chain variable region comprising the amino acid sequence set forth in SEQ ID NO: 8, SEQ ID NO: 18, or SEQ ID NO: 25.

In certain embodiments, the extracellular antigen-binding domain comprises:
(a) a heavy chain variable region comprising the amino acid sequence set forth in SEQ ID NO: 7, and a light chain variable region comprising the amino acid sequence set forth in SEQ ID NO: 8;
(b) a heavy chain variable region comprising the amino acid sequence set forth in SEQ ID NO: 17, and a light chain variable region comprising the amino acid sequence set forth in SEQ ID NO: 18;
(c) a heavy chain variable region comprising the amino acid sequence set forth in SEQ ID NO: 24, and a light chain variable region comprising the amino acid sequence set forth in SEQ ID NO: 25;
(d) a heavy chain variable region comprising the amino acid sequence set forth in SEQ ID NO: 34, and a light chain variable region comprising the amino acid sequence set forth in SEQ ID NO: 35;
(e) a heavy chain variable region comprising the amino acid sequence set forth in SEQ ID NO: 42, and a light chain variable region comprising the amino acid sequence set forth in SEQ ID NO: 43;
(f) a heavy chain variable region comprising the amino acid sequence set forth in SEQ ID NO: 52, and a light chain variable region comprising the amino acid sequence set forth in SEQ ID NO: 53;
(g) a heavy chain variable region comprising the amino acid sequence set forth in SEQ ID NO: 60, and a light chain variable region comprising the amino acid sequence set forth in SEQ ID NO: 61;
(h) a heavy chain variable region comprising the amino acid sequence set forth in SEQ ID NO: 66, and a light chain variable region comprising the amino acid sequence set forth in SEQ ID NO: 67;
(i) a heavy chain variable region comprising the amino acid sequence set forth in SEQ ID NO: 76, and a light chain variable region comprising the amino acid sequence set forth in SEQ ID NO: 77;
(j) a heavy chain variable region comprising the amino acid sequence set forth in SEQ ID NO: 83, and a light chain variable region comprising the amino acid sequence set forth in SEQ ID NO: 84;
(k) a heavy chain variable region comprising the amino acid sequence set forth in SEQ ID NO: 92, and a light chain variable region comprising the amino acid sequence set forth in SEQ ID NO: 93;
(l) a heavy chain variable region comprising the amino acid sequence set forth in SEQ ID NO: 102, and a light chain variable region comprising the amino acid sequence set forth in SEQ ID NO: 103;
(m) a heavy chain variable region comprising the amino acid sequence set forth in SEQ ID NO: 108, and a light chain variable region comprising the amino acid sequence set forth in SEQ ID NO: 109;
(n) a heavy chain variable region comprising the amino acid sequence set forth in SEQ ID NO: 108, and a light chain variable region comprising the amino acid sequence set forth in SEQ ID NO: 113;
(o) a heavy chain variable region comprising the amino acid sequence set forth in SEQ ID NO: 119, and a light chain variable region comprising the amino acid sequence set forth in SEQ ID NO: 120;
(p) a heavy chain variable region comprising the amino acid sequence set forth in SEQ ID NO: 126, and a light chain variable region comprising the amino acid sequence set forth in SEQ ID NO: 127;
(q) a heavy chain variable region comprising the amino acid sequence set forth in SEQ ID NO: 131, and a light chain variable region comprising the amino acid sequence set forth in SEQ ID NO: 132;
(r) a heavy chain variable region comprising the amino acid sequence set forth in SEQ ID NO: 141, and a light chain variable region comprising the amino acid sequence set forth in SEQ ID NO: 142;
(s) a heavy chain variable region comprising the amino acid sequence set forth in SEQ ID NO: 147, and a light chain variable region comprising the amino acid sequence set forth in SEQ ID NO: 148;
(t) a heavy chain variable region comprising the amino acid sequence set forth in SEQ ID NO: 153, and a light chain variable region comprising the amino acid sequence set forth in SEQ ID NO: 154;
(u) a heavy chain variable region comprising the amino acid sequence set forth in SEQ ID NO: 157, and a light chain variable region comprising the amino acid sequence set forth in SEQ ID NO: 158;
(v) a heavy chain variable region comprising the amino acid sequence set forth in SEQ ID NO: 163, and a light chain variable region comprising the amino acid sequence set forth in SEQ ID NO: 164;
(w) a heavy chain variable region comprising the amino acid sequence set forth in SEQ ID NO: 172, and a light chain variable region comprising the amino acid sequence set forth in SEQ ID NO: 173;
(x) a heavy chain variable region comprising the amino acid sequence set forth in SEQ ID NO: 180, and a light chain variable region comprising the amino acid sequence set forth in SEQ ID NO: 181;
(y) a heavy chain variable region comprising the amino acid sequence set forth in SEQ ID NO: 190, and a light chain variable region comprising the amino acid sequence set forth in SEQ ID NO: 191;
(z) a heavy chain variable region comprising the amino acid sequence set forth in SEQ ID NO: 197, and a light chain variable region comprising the amino acid sequence set forth in SEQ ID NO: 198; or
(aa) a heavy chain variable region comprising the amino acid sequence set forth in SEQ ID NO: 205, and a light chain variable region comprising the amino acid sequence set forth in SEQ ID NO: 206.

In certain embodiments, the extracellular antigen-binding domain comprises: a heavy chain variable region comprising the amino acid sequence set forth in SEQ ID NO: 7, and a light chain variable region comprising the amino acid sequence set forth in SEQ ID NO: 8.

In certain embodiments, the extracellular antigen-binding domain comprises: a heavy chain variable region comprising the amino acid sequence set forth in SEQ ID NO: 17, and a light chain variable region comprising the amino acid sequence set forth in SEQ ID NO: 18.

In certain embodiments, the extracellular antigen-binding domain comprises: a heavy chain variable region comprising the amino acid sequence set forth in SEQ ID NO: 24, and a light chain variable region comprising the amino acid sequence set forth in SEQ ID NO: 25.

In certain embodiments, the extracellular antigen-binding domain comprises a linker between a heavy chain variable region and a light chain variable region of the extracellular antigen-binding domain. In certain embodiments, the linker comprises or consists of the amino acid sequence set forth in SEQ ID NO: 209, SEQ ID NO: 210, SEQ ID NO: 211, SEQ ID NO: 212, SEQ ID NO: 213, or SEQ ID NO: 214.

In certain embodiments, the extracellular antigen-binding domain comprises a signal peptide that is covalently joined to the 5′ terminus of the extracellular antigen-binding domain. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof. In certain embodiments, the intracellular signaling domain comprises a CD3ζ polypeptide. In certain embodiments, the CD3ζ polypeptide comprises or consists of the amino acid sequence set forth in SEQ ID NO: 221.

In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide. In certain embodiments, the CD28 polypeptide comprises or consists of amino acids 180 to 220 of SEQ ID NO: 7. In certain embodiments, the CD28 polypeptide comprises a mutated YMNM motif. In certain embodiments, the CD28 polypeptide comprises or consists of the amino acid sequence set forth in SEQ ID NO: 275, SEQ ID NO: 276, SEQ ID NO: 277, SEQ ID NO: 278, or SEQ ID NO: 279.

In certain embodiments, the antigen-recognizing receptor is a chimeric antigen receptor (CAR), or a T-cell like fusion protein. In certain embodiments, the antigen-recognizing receptor is a CAR.

In certain embodiments, the antigen-recognizing receptor is recombinantly expressed. In certain embodiments, the antigen-recognizing receptor is expressed from a vector. In certain embodiments, the vector is a γ-retroviral vector.

The presently disclosed subject matter provides cells comprises a presently disclosed antigen-recognizing receptor. In certain embodiments, the cell is transduced with the antigen-recognizing receptor. In certain embodiment, the antigen-recognizing receptor is constitutively expressed on the surface of the cell. In certain embodiment, the cell further comprises an exogenous IL-18 polypeptide. In certain embodiment, the exogenous IL-18 polypeptide is a human IL-18 polypeptide.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, and a stem cell from which lymphoid cells may be differentiated. In certain embodiments, the cell is a T cell. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell.

The presently disclosed subject matter further provides nucleic acids that encode a presently disclosed antigen-recognizing receptor. The presently disclosed subject matter further provides vectors comprising the presently disclosed nucleic acid molecules. In certain embodiments, the vector is a viral vector. In certain embodiments, the vector is a γ-retroviral vector.

In addition, the presently disclosed subject matter provides host cells expressing the nucleic acid molecule disclosed herein. In certain embodiments, the host cell is a T cell.

The presently disclosed subject matter further provides compositions comprising the cells disclosed herein. In certain embodiments, the composition is a pharmaceutical composition further comprising a pharmaceutically acceptable carrier.

The presently disclosed subject matter also provide lipid nanoparticles comprising nucleic acids that encode a presently disclosed antigen-recognizing receptor. Further, the presently disclosed subject matter provides composition comprising the lipid nanoparticles disclosed herein.

In certain embodiments, the composition is a pharmaceutical composition further comprising a pharmaceutically acceptable carrier.

The presently disclosed subject matter further provides methods of treating or ameliorating a disease or disorder in a subject. In certain embodiments, the method comprises administering to the subject the presently disclosed cells, or the compositions.

In certain embodiments, the disease or disorder expresses DLL3. In certain embodiments, the disease or disorder is associated with overexpression of DLL3. In certain embodiments, the disease or disorder is tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer (including typical carcinoid tumors, and atypical carcinoid tumors), large cell neuroendocrine carcinoma, and small-cell lung cancer. In certain embodiments, the tumor is small-cell lung cancer.

The presently disclosed subject matter further provides kits for treating or ameliorating a disease or disorder in a subject, comprising the presently disclosed cells, the nucleic acids, or the compositions. In certain embodiments, the kit further comprises written instructions for using the presently disclosed cell or composition for treating or ameliorating a disease or disorder in a subject.

In addition, the presently disclosed subject matter provides methods of producing a DLL3-targeted antigen-recognizing receptor, comprising introducing into the cell a nucleic acid that encodes the antigen-recognizing receptor.

Finally, the presently disclosed subject matter provides cells and/or compositions disclosed herein for use in treating or ameliorating a disease or disorder in a subject. In certain embodiments, the disease or disorder expresses DLL3. In certain embodiments, the disease or disorder is associated with overexpression of DLL3. In certain embodiments, the disease or disorder is tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer (including typical carcinoid tumors, and atypical carcinoid tumors), large cell neuroendocrine carcinoma, and small-cell lung cancer. In certain embodiments, the tumor is small-cell lung cancer.

### 4. BRIEF DESCRIPTION OF THE FIGURES

The following Detailed Description, given by way of example, but not intended to limit the invention to specific embodiments described, may be understood in conjunction with the accompanying drawings.

FIGS. 1A-1C depict overview of representative chimeric antigen receptors (CARs) disclosed herein. FIG. 1A shows a schematic overview of domains of the CARs disclosed herein, which are co-expressed with a truncated EGFR domain. FIG. 1B shows a schematic overview of the extracellular antigen-binding domain of three representative CARs disclosed herein, with a CD8 signal peptide and Flag tag for detection. VH: heavy chain; VL: light chain. FIG. 1C shows expression of the indicated CARs on transduced T cells measured by flow cytometry. Grey: untransduced T cells.

FIGS. 2A-2C depict functional activity of T cell expressing DLL3-targeted BBz CARs disclosed herein. FIG. 2A shows cytotoxicity of human T cells expressing antigen-recognizing receptors disclosed herein against DLL3⁺ small cell lung cancer cell lines H82 and H69. FIG. 2B shows a long-term proliferation assay. FIG. 2C shows secretion of pro-inflammatory cytokines by stimulated human T cells expressing DLL3-targeted CARs disclosed herein.

FIGS. 3A and 3B depict in vivo activity of T cell expressing DLL3-targeted 28z CARs disclosed herein. FIG. 3A shows tumor growth of metastatic H82-GFP-luciferase in NSG mice receiving the cells disclosed herein. FIG. 3B shows survival curve of NSG mice receiving the cells disclosed herein.

FIGS. 4A-4C depict activity of T cells expressing DLL3-targeted 28z CARs disclosed herein including IL-18 polypeptide. FIG. 4A shows a schematic overview of the retroviral vector with truncated EGFRt, 2J8-28z CAR, and exogenous IL-18 polypeptide. FIG. 4B shows CAR T cell proliferation in co-cultures with H82 or H69 SCLC cells (E:T ratio of 1:5). FIG. 4C shows average radiance of H82 tumor or H69 tumor in mice receiving T cells expressing DLL3-targeted 28z CARs disclosed herein including IL-18 polypeptide.

FIGS. 5A and 5B depict activity of T cells expressing DLL3-targeted 28z CARs disclosed herein including CD28 mutant polypeptide designated as “2J8-28YSNVz”. FIG. 5A shows effects T cells expressing the 2J8-28YSNVz CAR disclosed herein in mice having H82 tumor. FIG. 5B shows average radiance of metastatic SHP-77 SCLC tumors in mice receiving T cells expressing the 2J8-28YSNVz CAR disclosed herein and including exogenous IL-18 polypeptide. Cross indicates death due to GvHD.

### 5. DETAILED DESCRIPTION OF THE INVENTION

The presently disclosed subject matter provides antigen-recognizing receptors (e.g., chimeric antigen receptors (CARs)) that specifically target DLL3. The presently disclosed subject matter further provides cells comprising such receptors. The cells can be immunoresponsive cells, e.g., genetically modified immunoresponsive cells (e.g., T cells or NK cells). The presently disclosed subject matter also provides methods of using such cells for treatments, e.g., for treating and or ameliorating a disease or disorder associated with DLL3.

Non-limiting embodiments of the present disclosure are described by the present specification and Examples.

For purposes of clarity of disclosure and not by way of limitation, the detailed description is divided into the following subsections:
5.1. Definitions;
5.2. DLL3;
5.3. Antigen-Recognizing Receptors;
5.4. Cells;
5.5. Compositions and Vectors;
5.6. Polypeptides;
5.7. Formulations and Administration;
5.8. Methods of Treatment;
5.9. Kits; and
5.10. Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an exogenous CAR binds to an antigen, a formation of an immunological synapse occurs that includes clustering of many molecules near the bound receptor (e.g. CD4 or CD8, CD3γ/δ/εζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., DLL3). In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the IMGT numbering system. In certain embodiments, the CDR regions are delineated using the IMGT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises or consists of the amino acid sequence set forth in SEQ ID NO: 209, which is provided below:

[SEQ ID NO: 209]
GGGGSGGGGSGGGSGGGGS

In certain embodiments, the linker comprise or consists of the amino acid sequence set forth in SEQ ID NO: 210, which is provided below:

[SEQ ID NO: 210]
GSGGGGSGGGGS

In certain embodiments, the linker comprises or consists of the amino acid sequence set forth in SEQ ID NO: 211, which is provided below:

[SEQ ID NO: 211]
GGGGSGGGGSGGGGSGGGSGGGGS

In certain embodiments, the linker comprises or consists of the amino acid sequence set forth in SEQ ID NO: 212, which is provided below:

[SEQ ID NO: 212]
GGGGSGGGGSGGGGSGGGGSGGGSGGGGS

In certain embodiments, the linker comprises or consists of the amino acid sequence set forth in SEQ ID NO: 213, which is provided below:

[SEQ ID NO: 213]
GGGGS

In certain embodiments, the linker comprises or consists of the amino acid sequence set forth in SEQ ID NO: 214, which is provided below:

[SEQ ID NO: 214]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) (2008); 27(6):455-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife et al., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chem (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “a conservative sequence modification” refers to an amino acid modification that does not significantly affect or alter the binding characteristics of the presently disclosed DLL3-targeted CAR (e.g., the extracellular antigen-binding domain) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “recognize” is meant selectively binds to a target. A T cell that recognizes a tumor can expresses a receptor (e.g., a CAR) that binds to a tumor antigen.

By “signal sequence” or “leader sequence” is meant a peptide sequence (e.g., 5, 10, 15, 20, 25 or 30 amino acids) present at the N-terminus of newly synthesized proteins that directs their entry to the secretory pathway

By “specifically binds” or “specifically binds to” or “specifically target” is meant a polypeptide or a fragment thereof that recognizes and/or binds to a biological molecule of interest (e.g., a polypeptide, e.g., a DLL3 polypeptide), but which does not substantially recognize and/or bind other molecules in a sample, for example, a biological sample, which naturally includes a presently disclosed polypeptide (e.g., a DLL3 polypeptide).

As used herein, the term “derivative” refers to a compound that is derived from some other compound and maintains its general structure. For example, but without any limitation, trichloromethane (chloroform) is a derivative of methane.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter.

### 5.2. DLL3

DLL3 is selectively expressed in high grade pulmonary neuroendocrine tumors of the lung (LU-NETs) and other neuroendocrine cancers. Lu-NETs embrace a heterogeneous family of neoplasms classified into four histological variants, namely typical carcinoid (TC), atypical carcinoid (AC), large cell neuroendocrine carcinoma (LCNEC) and small cell lung carcinoma (SCLC). Increased expression of DLL3 was observed in SCLC and LCNEC patient-derived xenograft tumors and was also confirmed in primary tumors. See Saunders et al., Sci Translational Medicine (302): 302ra136 (2015). Both SCLC and pulmonary LCNEC are high-grade and poor-prognosis tumors, with higher incidence in smokers. Pulmonary LCNEC exhibits biologically aggressive behavior, similarly to SCLC. Stage by stage, survival curves of pulmonary LCNEC and SCLC overlap, and in addition, survival is lower than other NSCLCs. Prognosis is poor even in patients with potentially resectable stage I lung cancer with 5-year survival rates ranging from 27% to 67%. See Iyoda A. et al., J Thorac Cardiovasc Surg. 138:446-453 (2009).

Delta is one of the Drosophila ligands of Notch that activate signaling in adjacent cells. Humans have four known Notch receptors (NOTCH1 to NOTCH4), and three homologs of Delta, termed delta-like ligands: DLL1, DLL3 and DLL4. It has been reported that unlike DLL1 and DLL4, DLL3 inhibits Notch signaling rather than activating it.

DLL3 (also known as Delta-like 3 or SCDO1) is a member of the Delta-like family of Notch DSL ligands. Aberrant DLL3 expression (genotypic and/or phenotypic) is associated with various tumorigenic cell subpopulations such as cancer stem cells and tumor initiating cells.

In certain embodiments, the antigen recognizing receptor binds to human DLL3. In certain embodiments, the human DLL3 comprises or consists of the amino acid sequence with a UniProt Reference No: Q9NYJ7-1 (SEQ ID NO: 215) or a fragment thereof. SEQ ID NO: 215 is provided below. In certain embodiments, the DLL3 comprises an extracellular domain, a transmembrane domain, and a cytoplasmic domain. In certain embodiments, the extracellular domain comprises or consists of amino acids 27 to 492 of SEQ ID NO: 215. In certain embodiments, the transmembrane domain comprises or consists of amino acids 493 to 513 of SEQ ID NO: 215. In certain embodiments, the cytoplasmic domain comprises or consists of amino acids 514 to 618 of SEQ ID NO: 215.

In certain embodiments, the extracellular domain of DLL3 comprises a DSL domain, an EGF-like 1 domain, an EGF-like 2 domain, an EGF-like 3 domain, an EGF-like 4 domain, and EGF-like 5 domain, and an EGF-like 6 domain. In certain embodiments, the DSL domain comprises or consists of amino acids 176 to 215 of SEQ ID NO: 215. In certain embodiments, the EGF-like 1 domain comprises or consists of amino acids 216 to 249 of SEQ ID NO: 215. In certain embodiments, the EGF-like 2 domain comprises or consists of amino acids 274 to 310 of SEQ ID NO: 215. In certain embodiments, the EGF-like 3 domain comprises or consists of amino acids 312 to 351 of SEQ ID NO: 215. In certain embodiments, the EGF-like 4 domain comprises or consists of amino acids 353 to 389 of SEQ ID NO: 215. In certain embodiments, the EGF-like 5 domain comprises or consists of amino acids 391 to 427 of SEQ ID NO: 215. In certain embodiments, the EGF-like 6 domain comprises or consists of amino acids 429 to 465 of SEQ ID NO: 215.

[SEQ ID NO: 215]
MVSPRMSGLL SQTVILALIF LPQTRPAGVF ELQIHSFGPG
PGPGAPRSPC SARLPCRLFF RVCLKPGLSE EAAESPCALG
AALSARGPVY TEQPGAPAPD LPLPDGLLQV PERDAWPGTF
SFIIETWREE LGDQIGGPAW SLLARVAGRR RLAAGGPWAR
DIQRAGAWEL RESYRARCEP PAVGTACTRL CRPRSAPSRC
GPGLRPCAPL EDECEAPLVC RAGCSPEHGF CEQPGECRCL
EGWTGPLCTV PVSTSSCLSP RGPSSATTGC LVPGPGPCDG
NPCANGGSCS ETPRSFECTC PRGFYGLRCE VSGVTCADGP
CENGGLCVGG ADPDSAYICH CPPGFQGSNC EKRVDRCSLQ
PCRNGGLCLD LGHALRCRCR AGFAGPRCEH DLDDCAGRAC
ANGGTCVEGG GAHRCSCALG FGGRDCRERA DPCAARPCAH
GGRCYAHFSG LVCACAPGYM GARCEFPVHP DGASALPAAP
PGLRPGDPQR YLLPPALGLL VAAGVAGAAL LLVHVRRRGH
SQDAGSRLLA GTPEPSVHAL PDALNNLRTQ EGSGDGPSSS
VDWNRPEDVD PQGIYVISAP SIYAREVATP LFPPLHTGRA
GQRQHLLFPY PSSILSVK

In certain embodiments, the DLL3 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 215 or a fragment thereof.

In certain embodiments, the antigen recognizing receptor binds to a portion of human DLL3. In certain embodiments, the antigen recognizing receptor binds to the extracellular domain of DLL3. In certain embodiments, the antigen recognizing receptor binds to amino acids 27 to 492 of SEQ ID NO: 215.

In certain embodiments, the antigen recognizing receptor binds to EGF-like 3 domain of DLL3. In certain embodiments, the antigen recognizing receptor binds to amino acids 312 to 351 of SEQ ID NO: 215. In certain embodiments, the antigen recognizing receptor binds to EGF-like 4 domain of DLL3. In certain embodiments, the antigen recognizing receptor binds to amino acids 353 to 389 of SEQ ID NO: 215. In certain embodiments, the antigen recognizing receptor binds to EGF-like 5 domain of DLL3. In certain embodiments, the antigen recognizing receptor binds to amino acids 391 to 427 of SEQ ID NO: 215. In certain embodiments, the antigen recognizing receptor binds to EGF-like 6 domain of DLL3. In certain embodiments, the antigen recognizing receptor binds to amino acids 429 to 465 of SEQ ID NO: 215.

### 5.3. Antigen-Recognizing Receptors

The presently disclosed antigen-recognizing receptors specifically target or binds to DLL3. In certain embodiments, the antigen-recognizing receptor is a chimeric antigen receptor (CAR). In certain embodiments, the antigen-recognizing receptor is a TCR like fusion molecule.

The presently disclosed subject matter also provides nucleic acid molecules that encode the presently disclosed antigen-recognizing receptors. In certain embodiments, the nucleic acid molecule comprises a nucleotide sequence that encodes a polypeptide of a DLL3-targeted antigen recognizing receptor disclosed herein.

### 5.3.1. Extracellular Antigen-Binding Domains

In certain embodiments, the extracellular antigen-binding domain of the antigen-recognizing receptor binds to DLL3.

In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the scFv is identified by screening scFv phage library with an antigen-Fc fusion protein.

In certain embodiments, the extracellular antigen-binding domain is a Fab. In certain embodiments, the Fab is crosslinked. In certain embodiments, the extracellular antigen-binding domain is a F(ab)₂.

Any of the foregoing molecules may be comprised in a fusion protein with a heterologous sequence to form the extracellular antigen-binding domain. In certain embodiments, the extracellular antigen-binding domain (embodied, for example, an scFv) binds to DLL3 (e.g., human DLL3) with a binding affinity, for example with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10⁻¹² M or less, or about 1×10⁻¹² M or less. In certain embodiments, extracellular antigen-binding domain (embodied, for example, an scFv) binds to DLL3 (e.g., human DLL3) with a binding affinity, for example with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less, about 5×10⁻¹² M or less, or about 1×10⁻¹² M or less. In certain embodiments, extracellular antigen-binding domain (embodied, for example, an scFv) binds to DLL3 (e.g., human DLL3) with a binding affinity, for example with a dissociation constant (K_{D}) of about 5×10⁻⁹ M or less. In certain embodiments, extracellular antigen-binding domain (embodied, for example, an scFv) binds to DLL3 (e.g., human DLL3) with a binding affinity, for example with a dissociation constant (K_{D}) of about 1×10⁻⁹ M or less. In certain embodiments, extracellular antigen-binding domain (embodied, for example, an scFv) binds to DLL3 (e.g., human DLL3) with a binding affinity, for example with a dissociation constant (K_{D}) of about 3.5×10⁻⁹ M. In certain embodiments, extracellular antigen-binding domain (embodied, for example, an scFv) binds to DLL3 (e.g., human DLL3) with a binding affinity, for example with a dissociation constant (K_{D}) of about 1.5×10⁻⁹ M. In certain embodiments, extracellular antigen-binding domain (embodied, for example, an scFv) binds to DLL3 (e.g., human DLL3) with a binding affinity, for example with a dissociation constant (K_{D}) of about 1×10⁻¹² M.

Binding of the extracellular antigen-binding domain can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the DLL3-targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In certain embodiments, the DLL3-targeted human scFv is labeled with GFP.

In certain embodiments, the CDRs are identified according to the IMGT numbering system.

In certain embodiments, the extracellular antigen-binding domain (e.g., an scFv) comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOs: 1-3 are provided in Table 1.

In certain embodiments, the extracellular antigen-binding domain (e.g., an scFv) comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOs: 4-6 are provided in Table 1.

In certain embodiments, the extracellular antigen-binding domain (e.g., an scFv) comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and a V_{L} Comprising a CDR1 comprising the amino acid sequence set forth in SEQ ID NO: 4 or a conservative modification thereof, a CDR2 comprising the amino acid sequence set forth in SEQ ID NO: 5 or a conservative modification, and a CDR3 comprising the amino acid sequence set forth in SEQ ID NO: 6 or a conservative modification thereof.

In certain embodiments, the extracellular antigen-binding domain (e.g., an scFv) comprises a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 7. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 7. In certain embodiments, the extracellular antigen-binding domain comprises a V_{H} comprising the amino sequence set forth in SEQ ID NO: 7. An exemplary nucleotide sequence encoding the amino acid sequence of SEQ ID NO: 7 is set forth in SEQ ID NO: 9. SEQ ID NOs: 7 and 9 are provided in Table 1 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 8. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 8. In certain embodiments, the extracellular antigen-binding domain comprises a V_{L} comprising the amino sequence set forth in SEQ ID NO: 8. An exemplary nucleotide sequence encoding the amino acid sequence of SEQ ID NO: 8 is set forth in SEQ ID NO: 10. SEQ ID NOs: 8 and 10 are provided in Table 1 below.

In certain embodiments, the extracellular antigen-binding domain (e.g., an scFv) comprises a V_{H} comprising the amino acid sequence set forth in SEQ ID NO: 7, and a V_{L} comprising the amino acid sequence set forth in SEQ ID NO: 8. In certain embodiments, the extracellular antigen-binding domain is an scFv. In certain embodiments, the scFv is designated as “J8”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}.

TABLE 1

| CDRs | 1 | 2 | 3 |
|---|---|---|---|
| V_{H} | GYTFTIY | NPSGGS | NDYVDYVTTDYYGMDV |
| | [SEQ ID | [SEQ ID | [SEQ ID |
| | NO: 1] | NO: 2] | NO: 3] |
| V_{L} | RASQGISNYLA | AASSLQS | QQYNSSPYT |
| | [SEQ ID NO: 4] | [SEQ ID NO: 5] | [SEQ ID NO: 6] |

| | |
|---|---|
| Full V_{H} | QVHLVQSGAEVKKPGASVKVSCKASGYTFTIYYIHWVRQAPGQGLEWMGIINPS |
| | GGSTSYAQKFQGRVTMTRDTSTSTVYMELSSLRSEDTAVYYCSGNDYVDYVTTD |
| | YYGMDVWGQGTTVTVSS [SEQ ID NO: 7] |
| Full V_{L} | DIQMTQSPSSLSASVGDRVTITCRASQGISNYLAWFQQKPGKAPKSLIYAASSL |
| | QSGVPSKFSGSGSGTDFTLTISSLQPEDFATYYCQQYNSSPYTFGQGTKLEIK |
| | [SEQ ID NO: 8] |
| DNA for | CAGGTGCATCTGGTGCAGTCTGGGGCTGAGGTGAAGAAGCCTGGGGCCTCAGTG |
| Full V_{H} | AAGGTTTCTTGCAAGGCATCTGGATACACCTTCACCATCTACTATATTCACTGG |
| | GTGCGACAGGCCCCTGGACAGGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTATATTACTGTTCGGGTAATGACTACGTTGACTACGTAACTACTGAC |
| | TACTACGGTATGGACGTCTGGGGCCAAGGGACCACGGTCACCGTCTCCTCA |
| | [SEQ ID NO: 9] |
| DNA for | GACATCCAGATGACCCAGTCTCCATCCTCACTGTCTGCATCTGTAGGAGATAGA |
| Full V_{L} | GTCACCATCACTTGTCGGGCGAGTCAGGGCATTAGCAATTATTTAGCCTGGTTT |
| | CAGCAGAAACCAGGGAAAGCCCCTAAGTCCCTGATCTATGCTGCATCCAGTTTG |
| | CAAAGTGGGGTCCCATCAAAGTTCAGCGGCAGTGGATCTGGGACAGATTTCACA |
| | CTCACCATCAGCAGCCTGCAGCCTGAAGATTTTGCAACTTATTACTGCCAACAG |
| | TATAATAGTTCCCCGTACACTTTTGGCCAGGGGACCAAGCTGGAGATCAAA |
| | [SEQ ID NO: 10] |

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOs: 11-13 are provided in Table 2.

In certain embodiments, the extracellular antigen-binding domain (e.g., an scFv) comprises a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOs: 14-16 are provided in Table 2.

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; and a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 146 or a conservative modification thereof.

In certain embodiments, the extracellular antigen-binding domain (e.g., a scFv) comprises a V_{H} comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and a V_{L} comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 17. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 17. In certain embodiments, the extracellular antigen-binding domain comprises a V_{H} comprising the amino sequence set forth in SEQ ID NO: 17. An exemplary nucleotide sequence encoding the amino acid sequence of SEQ ID NO: 17 is set forth in SEQ ID NO: 19. SEQ ID NO: 17 and 19 are provided in Table 2 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 18. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 18. In certain embodiments, the extracellular antigen-binding domain comprises a V_{L} comprising the amino sequence set forth in SEQ ID NO: 18. An exemplary nucleotide sequence encoding the amino acid sequence of SEQ ID NO: 18 is set forth in SEQ ID NO: 20. SEQ ID NO: 16 and 20 are provided in Table 2 below.

In certain embodiments, the extracellular antigen-binding domain (e.g., an scFv) comprises a V_{H} comprising the amino acid sequence set forth in SEQ ID NO: 17, and a V_{L} comprising the amino acid sequence set forth in SEQ ID NO: 18. In certain embodiments, the extracellular antigen-binding domain is an scFv. In certain embodiments, the scFv is designated as “L22”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 2

| CDRs | 1 | 2 | 3 |
|---|---|---|---|
| V_{H} | GFTFSSY | SGRGGD | ERGLGFDY |
| | [SEQ ID NO: 11] | [SEQ ID NO: 12] | [SEQ ID NO: 13] |
| V_{L} | RSSQSLLDSDDGNTYLD | TLSYRAS | MQHLEFPLT |
| | [SEQ ID NO: 14] | [SEQ ID NO: 15] | [SEQ ID NO: 16] |

| | |
|---|---|
| Full V_{H} | EVQLLESGGGLVQPGGSLRLSCAASGFTFSSYAMGWVRQTPGKGLEWVSTISGR |
| | GGDTYYADSVKGRFTISRDNSKNTLYLQMNSLRAEDTAIYYCAKERGLGEDYWG |
| | QGTLVTVSS |
| | [SEQ ID NO: 17] |
| Full V_{L} | DIVMTQTPLSLPVTPGEPASISCRSSQSLLDSDDGNTYLDWYLQKPGQSPQLLI |
| | YTLSYRASGVPDRESGSGSGTDFTLKISRVEAEDVGVYYCMQHLEFPLTFGGGT |
| | KVEIK [SEQ ID NO: 18] |
| DNA for | GAGGTGCAGCTGTTGGAGTCTGGGGGAGGCTTGGTACAGCCTGGGGGGTCCCTG |
| Full V_{H} | AGACTCTCCTGTGCAGCCTCTGGATTCACCTTTAGCAGCTATGCCATGGGCTGG |
| | GTCCGCCAGACTCCAGGGAAGGGGCTGGAGTGGGTCTCAACTATTAGTGGTCGT |
| | GGTGGTGACACATACTACGCAGACTCCGTGAAGGGCCGGTTCACCATCTCCAGA |
| | GACAATTCCAAGAACACGCTGTATCTGCAAATGAACAGCCTGAGAGCCGAGGAC |
| | ACGGCCATATATTACTGTGCGAAAGAGAGGGGACTGGGGTTTGACTACTGGGGC |
| | CAGGGAACCCTGGTCACCGTCTCCTCA |
| | [SEQ ID NO: 19] |
| DNA for | GATATTGTGATGACCCAGACTCCACTCTCCCTGCCCGTCACCCCTGGAGAGCCG |
| Full V_{L} | GCCTCCATCTCCTGCAGGTCTAGTCAGAGCCTCTTGGATAGTGATGATGGAAAC |
| | ACCTATTTGGACTGGTACCTGCAGAAGCCAGGGCAGTCTCCACAGCTCCTGATC |
| | TATACGCTTTCCTATCGGGCCTCTGGAGTCCCAGACAGGTTCAGTGGCAGTGGG |
| | TCAGGCACTGATTTCACACTGAAAATCAGCAGGGTGGAGGCTGAGGATGTTGGA |
| | GTTTATTACTGCATGCAACATCTAGAGTTTCCGCTCACTTTCGGCGGAGGGACC |
| | AAGGTGGAGATCAAA |
| | [SEQ ID NO: 20] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof. SEQ ID NOS: 21, 2, and 22 are provided in Table 3.

In certain embodiments, the extracellular antigen-binding domain (e.g., an scFv) comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof. SEQ ID NOs: 4, 5, and 23 are provided in Table 3.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 2, a CDR3 comprising the amino acid sequence set forth in SEQ ID NO: 22; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 24. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 24. In certain embodiments, the extracellular antigen-binding domain comprises a V_{H} comprising the amino sequence set forth in SEQ ID NO: 24. An exemplary nucleotide sequence encoding the amino acid sequence of SEQ ID NO: 24 is set forth in SEQ ID NO: 26. SEQ ID NO: 24 and 26 are provided in Table 3 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 25. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 25. In certain embodiments, the extracellular antigen-binding domain comprises a V_{L} comprising the amino sequence set forth in SEQ ID NO: 25. An exemplary nucleotide sequence encoding the amino acid sequence of SEQ ID NO: 25 is set forth in SEQ ID NO: 27. SEQ ID NO: 25 and 27 are provided in Table 3 below.

In certain embodiments, the extracellular antigen-binding domain (e.g., an scFv) comprises a V_{H} comprising the amino acid sequence set forth in SEQ ID NO: 24, and a V_{L} comprising the amino acid sequence set forth in SEQ ID NO: 25. In certain embodiments, the extracellular antigen-binding domain is an scFv. In certain embodiments, the scFv is designated as “B2”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 3

| CDRs | 1 | 2 | 3 |
|---|---|---|---|
| V_{H} | GYTFTSY | NPSGGS | GDYDILTDYYYNMDV |
| | [SEQ ID NO: 21] | [SEQ ID NO: 2] | [SEQ ID NO: 22] |
| V_{L} | RASQGISNYLA | AASSLQS | LQHNSYPYT |
| | [SEQ ID NO: 4] | [SEQ ID NO: 5] | [SEQ ID NO: 23] |

| | |
|---|---|
| Full V_{H} | QVQLVQSGAEVKKPGASVKVSCKASGYTFTSYYMHWVRQAPGQGLEWMGIINPS |
| | GGSTSYAQKFRGRVTMTRDTSTSTVYMELSSLRSEDTAVYYCARGDYDILTDYY |
| | YNMDVWGQGTTVTVSS |
| | [SEQ ID NO: 24] |
| Full V_{L} | DIQMTQSPSAMSASVGDRVTITCRASQGISNYLAWFQQKPGKVPKRLIYAASSL |
| | QSGVPSRESGSGSGTEFTLTISSLQPEDFATYYCLQHNSYPYTFGQGTKLEIK |
| | [SEQ ID NO: 25] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCGGGGCAGAGTCACCATGACCAGG |
| | GACACGTCCACGAGCACAGTCTACATGGAGCTGAGCAGCCTGAGATCTGAGGAC |
| | ACGGCCGTTTATTACTGTGCGAGAGGGGATTACGATATTTTGACGGACTACTAC |
| | TACAATATGGACGTCTGGGGCCAAGGGACCACGGTCACCGTCTCCTCA |
| | [SEQ ID NO: 26] |
| DNA for | GACATCCAGATGACCCAGTCTCCATCTGCCATGTCTGCATCTGTAGGAGACAGA |
| Full V_{L} | GTCACCATCACTTGTCGGGCGAGTCAGGGCATTAGCAATTATTTAGCCTGGTTT |
| | CAGCAGAAACCAGGGAAAGTCCCTAAGCGCCTGATCTATGCTGCATCCAGTTTG |
| | CAAAGTGGGGTCCCATCAAGGTTCAGCGGCAGTGGATCTGGGACAGAATTCACT |
| | CTCACAATCAGCAGCCTGCAGCCTGAAGATTTTGCAACTTATTACTGTCTACAG |
| | CATAATAGTTACCCGTACACTTTCGGCCAGGGGACCAAGCTGGAGATCAAA |
| | [SEQ ID NO: 27] |

In certain embodiments, the extracellular antigen-binding domain (e.g., an scFv) comprises a V_{H} comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof. SEQ ID NOs: 28-30 are provided in Table 4.

In certain embodiments, the extracellular antigen-binding domain (e.g., an scFv) comprises a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID NOs: 31-33 are provided in Table 4.

In certain embodiments, the extracellular antigen-binding domain (e.g., an scFv) comprises a V_{H} comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof; and a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof.

In certain embodiments, the extracellular antigen-binding domain (e.g., an scFv) comprises a V_{H} comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a V_{L} comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 34. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 34. In certain embodiments, the extracellular antigen-binding domain comprises a V_{H} comprising the amino sequence set forth in SEQ ID NO: 34. An exemplary nucleotide sequence encoding the amino acid sequence of SEQ ID NO: 34 is set forth in SEQ ID NO: 36. SEQ ID NO: 34 and 36 are provided in Table 4 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 35. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 35. In certain embodiments, the extracellular antigen-binding domain comprises a V_{L} comprising the amino sequence set forth in SEQ ID NO: 35. An exemplary nucleotide sequence encoding the amino acid sequence of SEQ ID NO: 35 is set forth in SEQ ID NO: 37. SEQ ID NO: 35 and 37 are provided in Table 4 below.

In certain embodiments, the extracellular antigen-binding domain (e.g., an scFv) comprises a V_{H} comprising the amino acid sequence set forth in SEQ ID NO: 34, and a V_{L} comprising the amino acid sequence set forth in SEQ ID NO: 35. In certain embodiments, the extracellular antigen-binding domain is an scFv. In certain embodiments, the scFv is designated as “A18”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}—V_{H}.

TABLE 4

| CDRs | 1 | 2 | 3 |
|---|---|---|---|
| V_{H} | GFSLSTSGM | DWDDN | IRGYSGSYDAFDI |
| | [SEQ ID NO: 28] | [SEQ ID NO: 29] | [SEQ ID NO: 30] |
| V_{L} | RSSQSLLHSNGYNYLD | LGSNRAP | MQALQTPFT |
| | [SEQ ID NO: 31] | [SEQ ID NO: 32] | [SEQ ID NO: 33] |

| | |
|---|---|
| Full V_{H} | QVTLRESGPALVKSTQTLTLTCTFSGFSLSTSGMCVSWIRQTPGKALEWLALID |
| | WDDNKYYSTSLKTRLIISKDTSKNQVVLTMTNMEPVDTATYYCARIRGYSGSYD |
| | AFDIWGQGTMVTVSS |
| | [SEQ ID NO: 34] |
| Full V_{L} | DIVMTQSPLSLPVTPGEPASISCRSSQSLLHSNGYNYLDWYLQKPGQSPHILLY |
| | LGSNRAPGVPDRFSGSGSGTDFTLKISRVEAEDVGVYYCMQALQTPFTFGPGTK |
| | VDIK |
| | [SEQ ID NO: 35] |
| DNA for | CAGGTCACCTTGAGGGAGTCTGGTCCTGCGCTGGTGAAATCCACACAGACCCTC |
| Full V_{H} | ACACTGACCTGCACCTTCTCTGGGTTCTCACTCAGTACTAGTGGAATGTGTGTG |
| | AGCTGGATCCGTCAGACCCCAGGGAAGGCCCTGGAGTGGCTTGCACTCATTGAT |
| | TGGGATGATAATAAATACTACAGCACATCTCTGAAGACCAGGCTCATCATCTCC |
| | AAGGACACCTCCAAAAACCAGGTGGTCCTTACAATGACCAATATGGAACCTGTG |
| | GACACAGCCACGTATTATTGTGCACGGATACGGGGGTATAGTGGGAGCTACGAT |
| | GCTTTTGATATCTGGGGCCAAGGGACAATGGTCACCGTCTCTTCA |
| | [SEQ ID NO: 36] |
| DNA for | GATATTGTGATGACTCAGTCTCCACTCTCCCTGCCCGTCACCCCTGGAGAGCCG |
| Full V_{L} | GCCTCCATCTCCTGCAGGTCTAGTCAGAGCCTCCTGCATAGTAATGGATACAAC |
| | TATTTGGATTGGTACCTGCAGAAGCCAGGGCAGTCTCCACACATCCTGCTCTAT |
| | TTGGGTTCTAATCGGGCCCCCGGGGTCCCTGACAGGTTCAGTGGCAGTGGATCA |
| | GGCACAGATTTTACACTGAAAATCAGCAGAGTGGAGGCTGAGGATGTTGGGGTT |
| | TATTACTGCATGCAAGCTCTACAAACTCCATTCACTTTCGGCCCTGGGACCAAA |
| | GTGGATATCAAA |
| | [SEQ ID NO: 37] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof. SEQ ID NOs: 21, 38, and 39 are provided in Table 5.

In certain embodiments, the extracellular antigen-binding domain (e.g., an scFv) comprises a V_{L} comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof. SEQ ID NOs: 40, 5, and 41 are provided in Table 5.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof; and a V_{L} comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39; and a V_{L} comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 42. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 42. In certain embodiments, the extracellular antigen-binding domain comprises a V_{H} comprising the amino sequence set forth in SEQ ID NO: 42. An exemplary nucleotide sequence encoding the amino acid sequence of SEQ ID NO: 42 is set forth in SEQ ID NO: 44. SEQ ID NO: 42 and 44 is provided in Table 5 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 43. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 43. In certain embodiments, the extracellular antigen-binding domain comprises a V_{L} comprising the amino sequence set forth in SEQ ID NO: 43. An exemplary nucleotide sequence encoding the amino acid sequence of SEQ ID NO: 43 is set forth in SEQ ID NO: 45. SEQ ID NO: 43 and 45 are provided in Table 5 below.

In certain embodiments, the extracellular antigen-binding domain (e.g., an scFv) comprises a V_{H} comprising the amino acid sequence set forth in SEQ ID NO: 42, and a V_{L} comprising the amino acid sequence set forth in SEQ ID NO: 43. In certain embodiments, the extracellular antigen-binding domain is an scFv. In certain embodiments, the scFv is designated as “E9”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 5

| CDRs | 1 | 2 | 3 |
|---|---|---|---|
| V_{H} | GYTFTSY | NSSGGS | GYCTNGVYYDEDEDY |
| | [SEQ ID NO: 21] | [SEQ ID NO: 38] | [SEQ ID NO: 39] |
| V_{L} | RASQGISHYLA | AASSLQS | QQYNSYPLT |
| | [SEQ ID NO: 40] | [SEQ ID NO: 5] | [SEQ ID NO: 41] |

| | |
|---|---|
| Full V_{H} | QVQLVQSGAEVKKPGASVKVSCKASGYTFTSYYMHWVRQAPGQGLEWMGVINSS |
| | GGSTSYAQKFQGRVTMTRDTSTSTVYMDLSSLRSEDTAVYYCARGYCTNGVYYD |
| | EDFDYWGQGTLVTVSS |
| | [SEQ ID NO: 42] |
| Full V_{L} | DIQMTQSPSSLSASVGDRVTITCRASQGISHYLAWFQQKPGKAPTSLIYAASSL |
| | QSGVPSKFSGSGSGTDFTLTISSLQPEDFATYYCQQYNSYPLTFGGGTKVEIK |
| | [SEQ ID NO: 43] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAGTAATCAATTCTAGT |
| | GGTGGTAGCACAAGCTACGCACAGAAGTTCCAGGGCAGAGTCACCATGACCAGG |
| | GACACGTCCACGAGCACAGTCTACATGGACCTGAGCAGCCTGAGATCTGAGGAC |
| | ACGGCCGTGTATTACTGTGCGAGAGGGTATTGTACTAATGGTGTTTACTATGAT |
| | GAGGACTTTGACTACTGGGGCCAGGGAACCCTGGTCACCGTCTCCTCA |
| | [SEQ ID NO: 44] |
| DNA for | GACATCCAGATGACCCAGTCTCCATCCTCACTGTCTGCATCTGTAGGAGACAGA |
| Full V_{L} | GTCACCATCACTTGTCGGGCGAGTCAGGGCATTAGCCATTATTTAGCCTGGTTT |
| | CAGCAGAAACCAGGGAAAGCCCCTACGTCCCTGATCTATGCTGCATCCAGTTTG |
| | CAAAGTGGGGTCCCATCAAAGTTCAGCGGCAGTGGATCTGGGACAGATTTCACT |
| | CTCACCATCAGCAGCCTGCAGCCTGAAGATTTTGCAACTTATTACTGCCAACAG |
| | TATAATAGTTACCCTCTCACTTTCGGCGGAGGGACCAAGGTGGAGATCAAA |
| | [SEQ ID NO: 45] |

In certain embodiments, the extracellular antigen-binding domain (e.g., an scFv) comprises a V_{H} comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof. SEQ ID NOs: 46-48 are provided in Table 6.

In certain embodiments, the extracellular antigen-binding domain (e.g., an scFv) comprises a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 49 or a conservative modification thereof. SEQ ID NOs: 49-51 are provided in Table 6.

In certain embodiments, the extracellular antigen-binding domain (e.g., an scFv) comprises a V_{H} comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof; and a V_{L} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof.

In certain embodiments, the extracellular antigen-binding domain (e.g., an scFv) comprises a V_{H} comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a V_{L} comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 52. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 52. In certain embodiments, the extracellular antigen-binding domain comprises a V_{H} comprising the amino sequence set forth in SEQ ID NO: 52. An exemplary nucleotide sequence encoding the amino acid sequence of SEQ ID NO: 52 is set forth in SEQ ID NO: 54. SEQ ID NO: 52 and 54 are provided in Table 6 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 53. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 51. In certain embodiments, the extracellular antigen-binding domain comprises a V_{L} comprising the amino sequence set forth in SEQ ID NO: 53. An exemplary nucleotide sequence encoding the amino acid sequence of SEQ ID NO: 53 is set forth in SEQ ID NO: 55. SEQ ID NO: 53 and 55 are provided in Table 6 below.

In certain embodiments, the extracellular antigen-binding domain (e.g., an scFv) comprises a V_{H} comprising the amino acid sequence set forth in SEQ ID NO: 52, and a V_{L} comprising the amino acid sequence set forth in SEQ ID NO: 53. In certain embodiments, the extracellular antigen-binding domain is an scFv. In certain embodiments, the scFv is designated as “G3”. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 6

| CDRs | 1 | 2 | 3 |
|---|---|---|---|
| V_{H} | GGSISSY | YTSGS | EDWESWTEDI |
| | [SEQ ID NO: 46] | [SEQ ID NO: 47] | [SEQ ID NO: 48] |
| V_{L} | RSGQSLVYSDGDTYLN | KVSNRDS | MQGTHWPLS |
| | [SEQ ID NO: 49] | [SEQ ID NO: 50] | [SEQ ID NO: 51] |

| | |
|---|---|
| Full V_{H} | QVQLQESGPGLVKPSETLFLTCTVSGGSISSYYWTWIRQPAGKGLEWIGRIYTS |
| | GSTNYNPSLKSRVTMSVDTSKNQFSLKLSSVTAADTAVYYCAREDWESWTEDIW |
| | GQGTMVTVSS |
| | [SEQ ID NO: 52] |
| Full V_{L} | DVVMTQSPLSLPVTLGQPASISCRSGQSLVYSDGDTYLNWFQQRPGQSPRRLIY |
| | KVSNRDSGVPDRFSGSGSGTDFTLKISRVEAEDVGVYYCMQGTHWPLSFGGGTK |
| | VEIK |
| | [SEQ ID NO: 53] |
| DNA for | CAGGTGCAGCTGCAGGAGTCGGGCCCAGGACTGGTGAAGCCTTCGGAGACCCTG |
| Full V_{H} | TTCCTCACCTGCACTGTCTCTGGTGGCTCCATCAGTAGTTACTACTGGACCTGG |
| | ATCCGGCAGCCCGCCGGGAAGGGACTGGAGTGGATTGGGCGTATCTATACCAGT |
| | GGGAGCACCAACTACAACCCCTCCCTCAAGAGTCGAGTCACCATGTCAGTAGAC |
| | ACGTCCAAGAACCAGTTCTCCCTGAAGCTGAGCTCTGTGACCGCCGCGGACACG |
| | GCCGTGTATTACTGTGCGAGAGAGGACTGGGAATCGTGGACTTTTGATATCTGG |
| | GGCCAAGGGACAATGGTCACCGTCTCTTCA |
| | [SEQ ID NO: 54] |
| DNA for | GATGTTGTGATGACTCAGTCTCCACTCTCCCTGCCCGTCACCCTTGGACAGCCG |
| Full V_{L} | GCCTCCATCTCCTGCAGGTCTGGTCAAAGCCTCGTTTACAGTGATGGAGACACC |
| | TACTTGAATTGGTTTCAGCAGAGGCCAGGCCAATCTCCAAGGCGCCTAATTTAT |
| | AAGGTTTCTAACCGGGACTCTGGGGTCCCAGACAGATTCAGCGGCAGTGGGTCA |
| | GGCACTGATTTCACACTGAAAATCAGCAGGGTGGAGGCTGAGGATGTTGGGGTT |
| | TATTACTGCATGCAAGGTACACACTGGCCGCTCTCTTTCGGCGGAGGGACCAAG |
| | GTGGAGATCAAA |
| | [SEQ ID NO: 55] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof. SEQ ID NOs: 21, 2, and 56 are provided in Table 7.

In certain embodiments, the extracellular antigen-binding domain (e.g., an scFv) comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 58 or a conservative modification thereof. SEQ ID NOs: 57-59 are provided in Table 7.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof.

In certain embodiments, the extracellular antigen-binding domain (e.g., an scFv) comprises a V_{H} comprising a CDR1 comprising the amino acid sequence set forth in SEQ ID NO: 214, a CDR2 comprising the amino acid sequence set forth in SEQ ID NO: 2, and a CDR3 comprising the amino acid sequence set forth in SEQ ID NO: 56; and a V_{L} Comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 60. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 59. In certain embodiments, the extracellular antigen-binding domain comprises a V_{H} comprising the amino sequence set forth in SEQ ID NO: 60. An exemplary nucleotide sequence encoding the amino acid sequence of SEQ ID NO: 60 is set forth in SEQ ID NO: 62. SEQ ID NO: 60 and 62 are provided in Table 7 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 61. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 61. In certain embodiments, the extracellular antigen-binding domain comprises a V_{L} comprising the amino sequence set forth in SEQ ID NO: 61. An exemplary nucleotide sequence encoding the amino acid sequence of SEQ ID NO: 61 is set forth in SEQ ID NO: 63. SEQ ID NO: 61 and 63 are provided in Table 7 below.

In certain embodiments, the extracellular antigen-binding domain (e.g., an scFv) comprises a V_{H} comprising the amino acid sequence set forth in SEQ ID NO: 60, and a V_{L} comprising the amino acid sequence set forth in SEQ ID NO: 61. In certain embodiments, the extracellular antigen-binding domain is an scFv. In certain embodiments, the scFv is designated as “M11”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 7

| CDRs | 1 | 2 | 3 |
|---|---|---|---|
| V_{H} | GYTFTSY | NPSGGS | QLELLGNAFDI |
| | [SEQ ID NO: 21] | [SEQ ID NO: 2] | [SEQ ID NO: 56] |
| V_{L} | RASQSVSSSYLA | GASSRAT | QQYGSSPYT |
| | [SEQ ID NO: 57] | [SEQ ID NO: 58] | [SEQ ID NO: 59] |

| | |
|---|---|
| Full V_{H} | QVQLVQSGAEVKKPGASVKVSCKASGYTFTSYYIHWVRQAPGQGLEWMGIINPS |
| | GGSKSYAQKFQGRVTMTRDTSTGTVYMELSSLRSEDTAVYYCAGQLELLGNAFD |
| | IWGQGTMVTVSS |
| | [SEQ ID NO: 60] |
| Full V_{L} | EIVLTQSPGTLSLSPGERATLSCRASQSVSSSYLAWYQQKPGQAPRLLIYGASS |
| | RATGIPDRESGSGSGTDFTLTISRLEPEDFAVYYCQQYGSSPYTFGQGTKLEIK |
| | [SEQ ID NO: 61] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATACACTGG |
| | GTGCGACAGGCCCCTGGACAAGGGCTTGAGTGGATGGGAATAATCAACCCTAGT |
| | GGTGGTAGTAAAAGCTACGCACAGAAGTTCCAGGGCAGAGTCACCATGACCAGG |
| | GACACGTCCACGGGAACAGTCTATATGGAGCTGAGCAGCCTGAGATCTGAGGAC |
| | ACGGCCGTGTATTACTGTGCGGGACAACTGGAACTTTTGGGGAATGCTTTTGAT |
| | ATCTGGGGCCAAGGGACAATGGTCACCGTCTCTTCA |
| | [SEQ ID NO: 62] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCGTACACTTTTGGCCAGGGGACCAAGCTGGAGATCAAA |
| | [SEQ ID NO: 63] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 64 or a conservative modification thereof. SEQ ID NOs: 21, 2, and 64 are provided in Table 8.

In certain embodiments, the extracellular antigen-binding domain (e.g., an scFv) comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof. SEQ ID NOs: 4, 5, and 65 are provided in Table 8.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 64 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 66. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 66. In certain embodiments, the extracellular antigen-binding domain comprises a V_{H} comprising the amino sequence set forth in SEQ ID NO: 66. An exemplary nucleotide sequence encoding the amino acid sequence of SEQ ID NO: 66 is set forth in SEQ ID NO: 68. SEQ ID NO: 66 and 68 are provided in Table 8 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 67. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 67. In certain embodiments, the extracellular antigen-binding domain comprises a V_{L} comprising the amino sequence set forth in SEQ ID NO: 67. An exemplary nucleotide sequence encoding the amino acid sequence of SEQ ID NO: 67 is set forth in SEQ ID NO: 69. SEQ ID NO: 67 and 69 are provided in Table 8 below.

In certain embodiments, the extracellular antigen-binding domain (e.g., an scFv) comprises a V_{H} comprising the amino acid sequence set forth in SEQ ID NO: 66, and a V_{L} comprising the amino acid sequence set forth in SEQ ID NO: 67. In certain embodiments, the extracellular antigen-binding domain is an scFv. In certain embodiments, the scFv is designated as “024”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 8

| CDRs | 1 | 2 | 3 |
|---|---|---|---|
| V_{H} | GYTFTSY | NPSGGS | ERQQLDYYYFAMDV |
| | [SEQ ID NO: 21] | [SEQ ID NO: 2] | [SEQ ID NO: 64] |
| V_{L} | RASQGISNYLA | AASSLQS | QQYNSYPPT |
| | [SEQ ID NO: 4] | [SEQ ID NO: 5] | [SEQ ID NO: 65] |

| | |
|---|---|
| Full V_{H} | QVQLVQSGAEVKKPGASVKVSCKASGYTFTSYYLHWVRQAPGHGLEWMAIINPSGGSTS |
| | YAQKFLGRVTMTRDTSTNTVYMELSSLRSEDTAVYYCARERQQLDYYYFAMDVWGQGTT |
| | VTVSS |
| | [SEQ ID NO: 66] |
| Full V_{L} | DIQMTQSPSSLSASVGDRVTITCRASQGISNYLAWFQQKPGKAPKSLIYAASSLQSGVP |
| | SKFSGSGSGTDFTLTISSLQPEDFATYYCQQYNSYPPTFGQGTKVEIK |
| | [SEQ ID NO: 67] |
| DNA for | CAGGTGCAGCTGGTGCAGTCTGGGGCTGAGGTGAAGAAGCCTGGGGCCTCAGTGAAGGT |
| Full V_{H} | TTCCTGCAAGGCATCTGGATACACCTTCACCAGCTACTATTTGCACTGGGTGCGACAGG |
| | CCCCTGGACACGGGCTTGAGTGGATGGCAATAATCAACCCTAGTGGTGGTAGTACAAGC |
| | TACGCACAGAAGTTCCTGGGCAGAGTCACCATGACCAGGGACACGTCCACGAACACAGT |
| | CTACATGGAGCTGAGCAGCCTGAGATCTGAGGACACGGCCGTGTATTACTGTGCGAGAG |
| | AACGGCAGCAGCTGGACTACTACTACTTCGCTATGGACGTCTGGGGCCAAGGGACCACG |
| | GTCACCGTCTCCTCA |
| | [SEQ ID NO: 68] |
| DNA for | GACATCCAGATGACCCAGTCTCCATCCTCACTGTCTGCATCTGTAGGAGACAGAGTCAC |
| Full V_{L} | CATCACTTGTCGGGCGAGTCAGGGCATTAGCAATTATTTAGCCTGGTTTCAGCAGAAAC |
| | CAGGGAAAGCCCCTAAGTCCCTGATCTATGCTGCATCCAGTTTGCAAAGTGGGGTCCCA |
| | TCAAAGTTCAGCGGCAGTGGATCTGGGACAGATTTCACTCTCACCATCAGCAGCCTGCA |
| | GCCTGAAGATTTTGCAACTTATTACTGCCAACAGTATAATAGTTACCCTCCGACGTTCG |
| | GCCAAGGGACCAAGGTGGAAATCAAA |
| | [SEQ ID NO: 69] |

In certain embodiments, the extracellular antigen-binding domain (e.g., an scFv) comprises a V_{H} comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof. SEQ ID NOs: 70-72 are provided in Table 9.

In certain embodiments, the extracellular antigen-binding domain (e.g., an scFv) comprises a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof. SEQ ID NOs: 73-75 are provided in Table 9.

In certain embodiments, the extracellular antigen-binding domain (e.g., an scFv) comprises a V_{H} comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof; and a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof.

In certain embodiments, the extracellular antigen-binding domain (e.g., an scFv) comprises a V_{H} comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a V_{L} comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 76. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 76. In certain embodiments, the extracellular antigen-binding domain comprises a V_{H} comprising the amino sequence set forth in SEQ ID NO: 76. An exemplary nucleotide sequence encoding the amino acid sequence of SEQ ID NO: 76 is set forth in SEQ ID NO: 78. SEQ ID NO: 76 and 78 are provided in Table 9 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 77. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 77. In certain embodiments, the extracellular antigen-binding domain comprises a V_{L} comprising the amino sequence set forth in SEQ ID NO: 77. An exemplary nucleotide sequence encoding the amino acid sequence of SEQ ID NO: 77 is set forth in SEQ ID NO: 79. SEQ ID NO: 77 and 79 are provided in Table 9 below.

In certain embodiments, the extracellular antigen-binding domain (e.g., an scFv) comprises a V_{H} comprising the amino acid sequence set forth in SEQ ID NO: 76, and a V_{L} comprising the amino acid sequence set forth in SEQ ID NO: 77. In certain embodiments, the extracellular antigen-binding domain is an scFv. In certain embodiments, the scFv is designated as “P4”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 9

| CDRs | 1 | 2 | 3 |
|---|---|---|---|
| V_{H} | GFTFSFY | SSSSSY | NYDEWRGAFDI |
| | [SEQ ID NO: 70] | [SEQ ID NO: 71] | [SEQ ID NO: 72] |
| V_{L} | RASQSVSSNLA | GASTRAT | QQYNNWPTWT |
| | [SEQ ID NO: 73] | [SEQ ID NO: 74] | [SEQ ID NO: 75] |

| | |
|---|---|
| Full V_{H} | EVQLVESGGGLVKPGGSLRLSCAASGFTFSFYSMNWVRQAPGKGLEWVSSISSS |
| | SSYIYYADSVKGRFTISRDNAKNSLYLQMNSLRAEDTAVYYCARNYDFWRGAFD |
| | IWGQGTMVTVSS |
| | [SEQ ID NO: 76] |
| Full V_{L} | EIVMTQSPATLSVSPGERATLSCRASQSVSSNLAWYQQKPGQAPRLLIYGASTR |
| | ATGIPARFSGSGSGTEFTLTISSLQSEDFAVYYCQQYNNWPTWTFGQGTKVEIK |
| | [SEQ ID NO: 77] |
| DNA for | GAGGTGCAGCTGGTGGAGTCTGGGGGGGGCCTGGTCAAGCCTGGAGGGTCCCTG |
| Full V_{H} | AGACTCTCCTGTGCAGCCTCTGGATTCACCTTCAGTTTCTATAGCATGAACTGG |
| | GTCCGCCAGGCTCCAGGGAAGGGGCTGGAGTGGGTCTCATCCATTAGTAGTAGT |
| | AGTAGTTACATATACTACGCAGACTCAGTGAAGGGCCGATTCACCATCTCCAGA |
| | GACAACGCCAAGAACTCACTGTATCTGCAAATGAACAGCCTGAGAGCCGAGGAC |
| | ACGGCTGTGTATTACTGTGCGAGAAATTACGATTTTTGGAGAGGTGCTTTTGAT |
| | ATCTGGGGCCAAGGGACAATGGTCACCGTCTCTTCA |
| | [SEQ ID NO: 78] |
| DNA for | GAAATAGTGATGACGCAGTCTCCAGCCACCCTGTCTGTGTCTCCAGGGGAAAGA |
| Full V_{L} | GCCACCCTCTCCTGCAGGGCCAGTCAGAGTGTTAGCAGCAACTTAGCCTGGTAC |
| | CAGCAGAAACCTGGCCAGGCTCCCAGGCTCCTCATCTATGGTGCATCCACCAGG |
| | GCCACTGGTATCCCAGCCAGGTTCAGTGGCAGTGGGTCTGGGACAGAGTTCACT |
| | CTCACCATCAGCAGCCTGCAGTCTGAAGATTTTGCAGTTTATTACTGTCAGCAG |
| | TATAATAACTGGCCCACGTGGACGTTCGGCCAAGGGACCAAGGTGGAAATCAAA |
| | [SEQ ID NO: 79] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof. SEQ ID NOs: 21, 80, and 81 are provided in Table 10.

In certain embodiments, the extracellular antigen-binding domain (e.g., an scFv) comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 10.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 80 or a conservative modification thereof, and a CDR3 comprising the amino acid sequence set forth in SEQ ID NO: 81 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 83. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 83. In certain embodiments, the extracellular antigen-binding domain comprises a V_{H} comprising the amino sequence set forth in SEQ ID NO: 83. An exemplary nucleotide sequence encoding the amino acid sequence of SEQ ID NO: 83 is set forth in SEQ ID NO: 85. SEQ ID NO: 83 and 85 are provided in Table 10 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 84. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 84. In certain embodiments, the extracellular antigen-binding domain comprises a V_{L} comprising the amino sequence set forth in SEQ ID NO: 84. An exemplary nucleotide sequence encoding the amino acid sequence of SEQ ID NO: 84 is set forth in SEQ ID NO: 86. SEQ ID NO: 84 and 86 are provided in Table 10 below.

In certain embodiments, the extracellular antigen-binding domain (e.g., an scFv) comprises a V_{H} comprising the amino acid sequence set forth in SEQ ID NO: 83, and a V_{L} comprising the amino acid sequence set forth in SEQ ID NO: 84. In certain embodiments, the extracellular antigen-binding domain is an scFv. In certain embodiments, the scFv is designated as “J23”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 10

| CDRs | 1 | 2 | 3 |
|---|---|---|---|
| V_{H} | GYTFTSY | NYSGGS | QGRYSGSYLDY |
| | [SEQ ID NO: 21] | [SEQ ID NO: 80] | [SEQ ID NO: 81] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTSYYMHWVRQAPGQGLEWMGIINYS |
| | GGSKSYAQKFQGRVTMTRDTSTSTVYMELSSLRSEDTGVYYCARQGRYSGSYLD |
| | YWGQGTLVTVSS |
| | [SEQ ID NO: 83] |
| Full V_{L} | EIVLTQSPGTLSLSPGERATLSCRASQSVSSSYLAWYQQKPGQAPRLLIYGASS |
| | RATGIPDRESGSGSGTDETLTISRLEPEDFAVYYCQQYGSSPFGQGTKLEIK |
| | [SEQ ID NO: 84] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACTATAGT |
| | GGTGGTAGCAAAAGCTACGCACAGAAGTTCCAGGGCAGAGTCACCATGACCCGG |
| | GACACGTCCACGAGCACAGTCTACATGGAGCTGAGCAGCCTGAGATCTGAGGAC |
| | ACGGGCGTGTATTACTGTGCGAGACAGGGGCGGTATAGTGGGAGCTACCTTGAC |
| | TACTGGGGCCAGGGAACCCTGGTCACCGTCTCCTCA |
| | [SEQ ID NO: 85] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCTTTTGGCCAGGGGACCAAGCTGGAGATCAAA |
| | [SEQ ID NO: 86] |

In certain embodiments, the extracellular antigen-binding domain (e.g., an scFv) comprises a V_{H} comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof. SEQ ID NOs:87-89 are provided in Table 11.

In certain embodiments, the extracellular antigen-binding domain (e.g., an scFv) comprises a V_{L} comprising a CDR1 comprising the amino acid sequence set forth in SEQ ID NO: 90 or a conservative modification thereof, a CDR2 comprising the amino acid sequence set forth in SEQ ID NO: 280 or a conservative modification thereof, and a CDR3 comprising the amino acid sequence set forth in SEQ ID NO: 91 or a conservative modification thereof. SEQ ID NOs: 90, 280, and 91 are provided in Table 11.

In certain embodiments, the extracellular antigen-binding domain (e.g., an scFv) comprises a V_{H} comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a V_{H} CDR3 comprising the amino acid sequence set forth in SEQ ID NO: 89 or a conservative modification thereof; and a V_{L} comprising a CDR1 comprising the amino acid sequence set forth in SEQ ID NO: 90 or a conservative modification thereof, a CDR2 comprising the amino acid sequence set forth in SEQ ID NO: 280 or a conservative modification thereof, and a CDR3 comprising the amino acid sequence set forth in SEQ ID NO: 91 or a conservative modification thereof.

In certain embodiments, the extracellular antigen-binding domain (e.g., an scFv) comprises a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} comprising a CDR1 comprising the amino acid sequence set forth in SEQ ID NO: 90, a CDR2 comprising the amino acid sequence set forth in SEQ ID NO: 280, and a CDR3 comprising the amino acid sequence set forth in SEQ ID NO: 91.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 92. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 92. In certain embodiments, the extracellular antigen-binding domain comprises a V_{H} comprising the amino sequence set forth in SEQ ID NO: 92. An exemplary nucleotide sequence encoding the amino acid sequence of SEQ ID NO: 92 is set forth in SEQ ID NO: 94. SEQ ID NO: 92 and 94 are provided in Table 11 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 93. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 93. In certain embodiments, the extracellular antigen-binding domain comprises a V_{L} comprising the amino sequence set forth in SEQ ID NO: 93. An exemplary nucleotide sequence encoding the amino acid sequence of SEQ ID NO: 93 is set forth in SEQ ID NO: 95. SEQ ID NO: 93 and 95 are provided in Table 11 below.

In certain embodiments, the extracellular antigen-binding domain (e.g., an scFv) comprises a V_{H} comprising the amino acid sequence set forth in SEQ ID NO: 92, and a V_{L} comprising the amino acid sequence set forth in SEQ ID NO: 93. In certain embodiments, the extracellular antigen-binding domain is an scFv. In certain embodiments, the scFv is designated as “K19”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 11

| CDRs | 1 | 2 | 3 |
|---|---|---|---|
| V_{H} | GYTFNSY | NPSGGT | DWGEQYGMDV |
| | [SEQ ID NO: 87] | [SEQ ID NO: 88] | [SEQ ID NO: 89] |
| V_{L} | RSSQSLLHSNGYNYLD | LGSNRAS | MQALQTPLT |
| | [SEQ ID NO: 90] | [SEQ ID NO: 280] | [SEQ ID NO: 91] |

| | |
|---|---|
| Full V_{H} | QVQLVQSGAEVKKPGASLKVSCKTSGYTENSYYMHWVRQAPGQGLEWMGINNPS |
| | GGTTSYAQKFQGRVTMTRDTSTSTVYMELSSLRSEDTAVYYCARDWGEQYGMDV |
| | WGQGTTVTVSS |
| | [SEQ ID NO: 92] |
| Full V_{L} | DIVMTQSPLSLPVTPGEPASISCRSSQSLLHSNGYNYLDWYLQKPGQSPQLLIY |
| | LGSNRASGVPDRFSGSGSGTDFTLKISRVEAEDVGVYYCMQALQTPLTFGGGTK |
| | VEIK |
| | [SEQ ID NO: 93] |
| DNA for | CAGGTGCAGCTGGTGCAGTCTGGGGCTGAGGTGAAGAAGCCTGGGGCCTCACTG |
| Full V_{H} | AAGGTTTCCTGCAAGACATCTGGATACACCTTCAACAGCTACTATATGCACTGG |
| | GTGCGACAGGCCCCTGGACAAGGGCTTGAGTGGATGGGAATAAACAACCCTAGT |
| | GGTGGTACCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGAGATTGGGGGGAGCAGTACGGTATGGACGTC |
| | TGGGGCCAAGGGACCACGGTCACCGTCTCCTCA |
| | [SEQ ID NO: 94] |
| DNA for | GATATTGTGATGACTCAGTCTCCACTCTCCCTGCCCGTCACCCCTGGAGAGCCG |
| Full V_{L} | GCCTCCATCTCCTGCAGGTCTAGTCAGAGCCTCCTGCATAGTAATGGATACAAC |
| | TATTTGGATTGGTACCTGCAGAAGCCAGGACAGTCTCCACAGCTCCTGATCTAT |
| | TTGGGTTCTAATCGGGCCTCCGGGGTCCCTGACAGGTTCAGTGGCAGTGGATCA |
| | GGCACAGATTTTACACTGAAAATCAGCAGAGTGGAGGCTGAGGATGTTGGGGTT |
| | TATTACTGCATGCAAGCTCTACAAACTCCGCTCACTTTCGGCGGAGGGACCAAG |
| | GTGGAGATCAAA |
| | [SEQ ID NO: 95] |

In certain embodiments, the extracellular antigen-binding domain (e.g., an scFv) comprises a V_{H} comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof. SEQ ID NOs: 96-98 are provided in Table 12.

In certain embodiments, the extracellular antigen-binding domain (e.g., an scFv) comprises a V_{L} comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof. SEQ ID NOs: 99-101 are provided in Table 12.

In certain embodiments, the extracellular antigen-binding domain (e.g., an scFv) comprises a V_{H} comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof; and a V_{L} comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof.

In certain embodiments, the extracellular antigen-binding domain (e.g., an scFv) comprises a V_{H} comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a V_{L} comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 102. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 102. In certain embodiments, the extracellular antigen-binding domain comprises a V_{H} comprising the amino sequence set forth in SEQ ID NO: 102. An exemplary nucleotide sequence encoding the amino acid sequence of SEQ ID NO: 102 is set forth in SEQ ID NO: 104. SEQ ID NO: 102 and 104 are provided in Table 12 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 103. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 103. In certain embodiments, the extracellular antigen-binding domain comprises a V_{L} comprising the amino sequence set forth in SEQ ID NO: 103. An exemplary nucleotide sequence encoding the amino acid sequence of SEQ ID NO: 103 is set forth in SEQ ID NO: 105. SEQ ID NO: 103 and 105 are provided in Table 12 below.

In certain embodiments, the extracellular antigen-binding domain (e.g., an scFv) comprises a V_{H} comprising the amino acid sequence set forth in SEQ ID NO: 102, and a V_{L} comprising the amino acid sequence set forth in SEQ ID NO: 103. In certain embodiments, the extracellular antigen-binding domain is an scFv. In certain embodiments, the scFv is designated as “N10”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 12

| CDRs | 1 | 2 | 3 |
|---|---|---|---|
| V_{H} | GYTFTGY | NPHSGA | DSSGWYGDAFDI |
| | [SEQ ID NO: 96] | [SEQ ID NO: 97] | [SEQ ID NO: 98] |
| V_{L} | RSSQSLVHSDGNTYLS | KISNRES | MQATQFPLT |
| | [SEQ ID NO: 99] | [SEQ ID NO: 100] | [SEQ ID NO: 101] |

| | |
|---|---|
| Full V_{H} | QVQLVQSGAEVKKPGASVKVSCKASGYTFTGYYMHWVRQAPGQGLEWMGWINPH |
| | SGAPNSAQKFQVRVTMTRDTSISTAYMELSRLRSDDTAVYYCARDSSGWYGDAF |
| | DIWGQGTMVTVSS |
| | [SEQ ID NO: 102] |
| Full V_{L} | DIVMTQTPLSSPVTLGQPASISCRSSQSLVHSDGNTYLSWLQQRPGQPPRLLIY |
| | KISNRFSGVPDRFSGSGAGTDFTLKISRVEAEDVGVYYCMQATQFPLTFGGGTK |
| | VEIK |
| | [SEQ ID NO: 103] |
| DNA for | CAGGTGCAGCTGGTGCAGTCTGGGGCTGAGGTGAAGAAGCCTGGGGCCTCAGTG |
| Full V_{H} | AAGGTCTCCTGCAAGGCTTCTGGATACACCTTCACCGGCTACTATATGCACTGG |
| | GTGCGACAGGCCCCTGGACAAGGGCTTGAGTGGATGGGATGGATCAACCCTCAC |
| | AGTGGTGCCCCAAACTCTGCACAGAAGTTTCAGGTCAGGGTCACCATGACCAGG |
| | GACACGTCCATCAGCACAGCCTACATGGAGCTGAGCAGGCTGAGATCTGACGAC |
| | ACGGCCGTGTATTACTGTGCGAGGGATAGCAGCGGCTGGTACGGTGATGCTTTT |
| | GATATCTGGGGCCAAGGGACAATGGTCACCGTCTCTTCA |
| | [SEQ ID NO: 104] |
| DNA for | GATATTGTGATGACCCAGACTCCACTCTCCTCACCTGTCACCCTTGGACAGCCG |
| Full V_{L} | GCCTCCATCTCCTGCAGGTCTAGTCAAAGCCTCGTACACAGTGATGGCAACACC |
| | TACTTGAGTTGGCTTCAGCAGAGGCCAGGCCAGCCTCCAAGACTCCTAATTTAT |
| | AAGATTTCTAACCGGTTCTCTGGGGTCCCAGACAGATTCAGTGGCAGTGGGGCA |
| | GGGACAGATTTCACACTGAAAATCAGCAGGGTGGAAGCTGAGGATGTCGGGGTT |
| | TATTACTGCATGCAAGCTACACAATTTCCGCTCACTTTCGGCGGAGGGACCAAG |
| | GTGGAGATCAAA |
| | [SEQ ID NO: 105] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 21, 106, and 107 are provided in Table 13.

In certain embodiments, the extracellular antigen-binding domain (e.g., an scFv) comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 13.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 108. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 108. In certain embodiments, the extracellular antigen-binding domain comprises a V_{H} comprising the amino sequence set forth in SEQ ID NO: 108. An exemplary nucleotide sequence encoding the amino acid sequence of SEQ ID NO: 108 is set forth in SEQ ID NO: 110. SEQ ID NO: 108 and 110 are provided in Table 13 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 109. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 109. In certain embodiments, the extracellular antigen-binding domain comprises a V_{L} comprising the amino sequence set forth in SEQ ID NO: 109. An exemplary nucleotide sequence encoding the amino acid sequence of SEQ ID NO: 109 is set forth in SEQ ID NO: 111. SEQ ID NO: 109 and 111 are provided in Table 13 below.

In certain embodiments, the extracellular antigen-binding domain (e.g., an scFv) comprises a V_{H} comprising the amino acid sequence set forth in SEQ ID NO: 108, and a V_{L} comprising the amino acid sequence set forth in SEQ ID NO: 109. In certain embodiments, the extracellular antigen-binding domain is an scFv. In certain embodiments, the scFv is designated as “B16-v1”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 13

| CDRs | 1 | 2 | 3 |
|---|---|---|---|
| V_{H} | GYTFTSY | DPSGGS | QGRYVGSYLDY |
| | [SEQ ID NO: 21] | [SEQ ID NO: 106] | [SEQ ID NO: 107] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTSYYMHWVRQAPGQGLEWMGIIDPS |
| | GGSTSYAQKFQGRVTMTRDTSTNTVYMDLSSLRSEDTAVYYCARQGRYVGSYLD |
| | YWGQGTLVTVSS |
| | [SEQ ID NO: 108] |
| Full V_{L} | EIVLTQSPGTLSLSPGERATLSCRASQSVSSSYLAWYQQKPGQAPRLLIYGASS |
| | RATGIPDRFSGSGSGTDFTLTISRLEPEDFAVYYCQQYGSSPFGQGTKLEIK |
| | [SEQ ID NO: 109] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCGACCCTAGT |
| | GGTGGTAGCACAAGCTACGCACAGAAGTTCCAGGGCAGAGTCACCATGACCAGG |
| | GACACGTCCACGAACACAGTCTACATGGACCTGAGCAGCCTGAGATCTGAGGAC |
| | ACGGCCGTGTATTACTGTGCGAGACAGGGGCGGTATGTTGGGAGCTACCTTGAC |
| | TACTGGGGCCAGGGAACCCTGGTCACCGTCTCCTCA |
| | [SEQ ID NO: 110] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCTTTTGGCCAGGGGACCAAGCTGGAGATCAAA |
| | [SEQ ID NO: 111] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof. SEQ ID NOs: 21, 106, and 107 are provided in Table 14.

In certain embodiments, the extracellular antigen-binding domain (e.g., an scFv) comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification thereof. SEQ ID NOs: 4, 5 and 112 are provided in Table 14.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a V_{L} Comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 108. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 108. In certain embodiments, the extracellular antigen-binding domain comprises a V_{H} comprising the amino sequence set forth in SEQ ID NO: 108. An exemplary nucleotide sequence encoding the amino acid sequence of SEQ ID NO: 108 is set forth in SEQ ID NO: 110. SEQ ID NO: 108 and 110 are provided in Table 14 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 113. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 113. In certain embodiments, the extracellular antigen-binding domain comprises a V_{L} comprising the amino sequence set forth in SEQ ID NO: 113. An exemplary nucleotide sequence encoding the amino acid sequence of SEQ ID NO: 113 is set forth in SEQ ID NO: 113. SEQ ID NO: 113 and 114 are provided in Table 14 below.

In certain embodiments, the extracellular antigen-binding domain (e.g., an scFv) comprises a V_{H} comprising the amino acid sequence set forth in SEQ ID NO: 108, and a V_{L} comprising the amino acid sequence set forth in SEQ ID NO: 113. In certain embodiments, the extracellular antigen-binding domain is an scFv. In certain embodiments, the scFv is designated as “B16-v2”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 14

| CDRs | 1 | 2 | 3 |
|---|---|---|---|
| V_{H} | GYTFTSY | DPSGGS | QGRYVGSYLDY |
| | [SEQ ID NO: 21] | [SEQ ID NO: 106] | [SEQ ID NO: 107] |
| V_{L} | RASQGISNYLA | AASSLQS | QQYNSYPYT |
| | [SEQ ID NO: 4] | [SEQ ID NO: 5] | [SEQ ID NO: 112] |

| | |
|---|---|
| Full V_{H} | QVQLVQSGAEVKKPGASVKVSCKASGYTFTSYYMHWVRQAPGQGLEWMGIIDPS |
| | GGSTSYAQKFQGRVTMTRDTSTNTVYMDLSSLRSEDTAVYYCARQGRYVGSYLD |
| | YWGQGTLVTVSS |
| | [SEQ ID NO: 108] |
| Full V_{L} | DIQMTQSPSSLSASVGDRVTITCRASQGISNYLAWFQQKPGKAPKSVIYAASSL |
| | QSGVPSKFSGSGSGTDFTLTISSMQPEDFATYYCQQYNSYPYTFGQGTKLEIK |
| | [SEQ ID NO: 113] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCGACCCTAGT |
| | GGTGGTAGCACAAGCTACGCACAGAAGTTCCAGGGCAGAGTCACCATGACCAGG |
| | GACACGTCCACGAACACAGTCTACATGGACCTGAGCAGCCTGAGATCTGAGGAC |
| | ACGGCCGTGTATTACTGTGCGAGACAGGGGCGGTATGTTGGGAGCTACCTTGAC |
| | TACTGGGGCCAGGGAACCCTGGTCACCGTCTCCTCA |
| | [SEQ ID NO: 110] |
| DNA for | GACATCCAGATGACCCAGTCTCCATCCTCACTGTCTGCATCTGTAGGAGACAGA |
| Full V_{L} | GTCACCATCACTTGTCGGGCGAGTCAGGGCATTAGCAATTATTTAGCCTGGTTT |
| | CAGCAGAAACCAGGGAAAGCCCCTAAGTCCGTGATCTATGCTGCATCCAGTTTG |
| | CAAAGTGGGGTCCCATCAAAGTTCAGCGGCAGTGGATCTGGGACAGATTTCACT |
| | CTCACCATCAGCAGCATGCAGCCTGAAGATTTTGCAACTTATTACTGCCAACAG |
| | TATAATAGTTACCCGTACACTTTTGGCCAGGGGACCAAGCTGGAGATCAAA |
| | [SEQ ID NO: 114] |

In certain embodiments, the extracellular antigen-binding domain (e.g., an scFv) comprises a V_{H} comprising a CDR1 comprising the amino acid sequence set forth in SEQ ID NO: 96 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof. SEQ ID NOs: 96, 115, and 116 are provided in Table 15.

In certain embodiments, the extracellular antigen-binding domain (e.g., an scFv) comprises a V_{L} comprising a CDR1 comprising the amino acid sequence set forth in SEQ ID NO: 117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification thereof. SEQ ID NOs: 117, 100, and 118 are provided in Table 15.

In certain embodiments, the extracellular antigen-binding domain (e.g., an scFv) comprises a V_{H} comprising a CDR1 comprising the amino acid sequence set forth in SEQ ID NO: 96 or a conservative modification thereof, a CDR2 comprising the amino acid sequence set forth in SEQ ID NO: 115 or a conservative modification thereof, and a CDR3 comprising the amino acid sequence set forth in SEQ ID NO: 116 or a conservative modification thereof; and a V_{L} comprising a CDR1 comprising the amino acid sequence set forth in SEQ ID NO: 117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification thereof.

In certain embodiments, the extracellular antigen-binding domain (e.g., an scFv) comprises a V_{H} comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116; and a V_{L} Comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 119. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 119. In certain embodiments, the extracellular antigen-binding domain comprises a V_{H} comprising the amino sequence set forth in SEQ ID NO: 119. An exemplary nucleotide sequence encoding the amino acid sequence of SEQ ID NO: 119 is set forth in SEQ ID NO: 121. SEQ ID NO: 119 and 121 are provided in Table 15 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 120. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 120. In certain embodiments, the extracellular antigen-binding domain comprises a V_{L} comprising the amino sequence set forth in SEQ ID NO: 120. An exemplary nucleotide sequence encoding the amino acid sequence of SEQ ID NO: 120 is set forth in SEQ ID NO: 122. SEQ ID NO: 120 and 122 are provided in Table 15 below.

In certain embodiments, the extracellular antigen-binding domain (e.g., an scFv) comprises a V_{H} comprising the amino acid sequence set forth in SEQ ID NO: 119, and a V_{L} comprising the amino acid sequence set forth in SEQ ID NO: 120. In certain embodiments, the extracellular antigen-binding domain is an scFv. In certain embodiments, the scFv is designated as “E23”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}—V_{H}.

TABLE 15

| CDRs | 1 | 2 | 3 |
|---|---|---|---|
| V_{H} | GYTFTGY | NSYSGG | DGSGWYGSYFDF |
| | [SEQ ID NO: 96] | [SEQ ID NO: 115] | [SEQ ID NO: 116] |
| V_{L} | RSSQSLVHSDGNTYLS | KISNRFS | IQTTQFPLT |
| | [SEQ ID NO: 117] | [SEQ ID NO: 100] | [SEQ ID NO: 118] |

| | |
|---|---|
| Full | QVQLVQSGAEVRKPGASVKVSCKASGYTFTGYYMHWVRQAPGQGLEWMGWINSYSG |
| V_{H} | GANYAQKFQDRVTMTRDTSVSTASMELSRLRSDDTAVYYCARDGSGWYGSYEDEWG |
| | QGTLVTVSS |
| | [SEQ ID NO: 119] |
| Full | DIVMTQTPLSSPVTLGQPASISCRSSQSLVHSDGNTYLSWLQQRPGQPPRLLIYKI |
| V_{L} | SNRFSGVPDRFSGSGAGTDFTLKISRVEAEDVGFYYCIQTTQFPLTFGGGTKVEIK |
| | [SEQ ID NO: 120] |
| DNA | CAGGTGCAGCTGGTGCAGTCTGGGGCTGAGGTGAGGAAGCCTGGGGCCTCAGTGAA |
| for | GGTCTCCTGCAAGGCTTCTGGATACACCTTCACCGGCTACTATATGCACTGGGTGC |
| Full | GACAGGCCCCTGGACAAGGGCTTGAGTGGATGGGATGGATCAACTCTTACAGTGGT |
| V_{H} | GGCGCAAACTATGCACAGAAGTTTCAGGACAGGGTCACCATGACCAGGGACACGTC |
| | CGTCAGCACAGCCTCCATGGAGCTGAGCAGGCTGAGATCTGACGACACGGCCGTGT |
| | ATTACTGTGCGAGAGATGGCAGTGGCTGGTACGGGTCCTACTTTGACTTCTGGGGC |
| | CAGGGAACCCTGGTCACCGTCTCCTCA |
| | [SEQ ID NO: 121] |
| DNA | GATATTGTGATGACCCAGACTCCACTCTCCTCACCTGTCACCCTTGGACAGCCGGC |
| for | CTCCATCTCCTGCAGGTCTAGTCAAAGCCTCGTACACAGTGATGGAAACACCTACT |
| Full | TGAGTTGGCTTCAGCAGAGGCCAGGCCAGCCTCCAAGACTCCTAATTTATAAGATT |
| V_{L} | TCTAACCGGTTCTCTGGGGTCCCAGACAGATTCAGTGGCAGTGGGGCAGGGACAGA |
| | TTTCACACTGAAAATCAGCAGGGTGGAAGCTGAGGATGTCGGGTTTTATTACTGCA |
| | TACAAACTACACAATTTCCGCTCACTTTCGGCGGAGGGACCAAGGTGGAGATCAAA |
| | [SEQ ID NO: 122] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof. SEQ ID NOs: 21, 2, and 123 are provided in Table 16.

In certain embodiments, the extracellular antigen-binding domain (e.g., an scFv) comprises a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification thereof. SEQ ID NOs: 124, 58, and 125 are provided in Table 16.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof; and a V_{L} Comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a V_{L} comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 126. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 126. In certain embodiments, the extracellular antigen-binding domain comprises a V_{H} comprising the amino sequence set forth in SEQ ID NO: 126. An exemplary nucleotide sequence encoding the amino acid sequence of SEQ ID NO: 126 is set forth in SEQ ID NO: 128. SEQ ID NO: 126 and 128 are provided in Table 16 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 127. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 127. In certain embodiments, the extracellular antigen-binding domain comprises a V_{L} comprising the amino sequence set forth in SEQ ID NO: 127. An exemplary nucleotide sequence encoding the amino acid sequence of SEQ ID NO: 127 is set forth in SEQ ID NO: 129. SEQ ID NO: 127 and 129 are provided in Table 16 below.

In certain embodiments, the extracellular antigen-binding domain (e.g., an scFv) comprises a V_{H} comprising the amino acid sequence set forth in SEQ ID NO: 126, and a V_{L} comprising the amino acid sequence set forth in SEQ ID NO: 127. In certain embodiments, the extracellular antigen-binding domain is an scFv. In certain embodiments, the scFv is designated as “F9”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 16

| CDRs | 1 | 2 | 3 |
|---|---|---|---|
| V_{H} | GYTFTSY | NPSGGS | QGGAQWLVLAFDI |
| | [SEQ ID NO: 21] | [SEQ ID NO: 2] | [SEQ ID NO: 123] |
| V_{L} | RASQSVSSYLA | GASSRAT | QQYDSSPYT |
| | [SEQ ID NO: 124] | [SEQ ID NO: 58] | [SEQ ID NO: 125] |

| | |
|---|---|
| Full V_{H} | QVQLVQSGAEVKKPGASVQVSCKASGYTFTSYYMHWVRQAPGQGLEWMGIINPS |
| | GGSTSYAQKFQGRVTMTRDTSTSTVYMELSSLRSEDTAVYYCARQGGAQWLVLA |
| | FDIWGQGTMVTVSS |
| | [SEQ ID NO: 126] |
| Full V_{L} | EIVLTQSPGTLSLSPGERATLSCRASQSVSSYLAWYQQKPGQPPRLLIYGASSR |
| | ATGIPDRFSGGGSGTDFTLTISRLEPEDFAVYYCQQYDSSPYTFGQGTKLEIK |
| | [SEQ ID NO: 127] |
| DNA for | CAGGTGCAGCTGGTGCAGTCTGGGGCTGAGGTGAAGAAGCCTGGGGCCTCAGTG |
| Full V_{H} | CAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGACAGGGGGGAGCCCAGTGGCTGGTACTTGCT |
| | TTTGATATCTGGGGCCAAGGGACAATGGTCACCGTCTCTTCA |
| | [SEQ ID NO: 128] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCTACTTAGCCTGGTAC |
| | CAGCAGAAACCTGGCCAGCCTCCCAGGCTCCTCATCTATGGTGCATCCAGCAGG |
| | GCCACTGGCATCCCAGACAGGTTCAGTGGCGGTGGGTCTGGGACAGACTTCACT |
| | CTCACCATCAGCAGACTGGAGCCTGAAGATTTTGCAGTGTATTACTGTCAGCAG |
| | TATGATAGCTCACCGTACACTTTTGGCCAGGGGACCAAGCTGGAGATCAAA |
| | [SEQ ID NO: 129] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof. SEQ ID NOs: 21, 2, and 56 are provided in Table 17.

In certain embodiments, the extracellular antigen-binding domain (e.g., an scFv) comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification thereof. SEQ ID NOs: 57, 58, and 130 are provided in Table 17.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 131. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 131. In certain embodiments, the extracellular antigen-binding domain comprises a V_{H} comprising the amino sequence set forth in SEQ ID NO: 131. An exemplary nucleotide sequence encoding the amino acid sequence of SEQ ID NO: 131 is set forth in SEQ ID NO: 133. SEQ ID NO: 131 and 133 are provided in Table 17 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 132. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 132. In certain embodiments, the extracellular antigen-binding domain comprises a V_{L} comprising the amino sequence set forth in SEQ ID NO: 132. An exemplary nucleotide sequence encoding the amino acid sequence of SEQ ID NO: 132 is set forth in SEQ ID NO: 134. SEQ ID NO: 132 and 134 are provided in Table 17 below.

In certain embodiments, the extracellular antigen-binding domain (e.g., an scFv) comprises a V_{H} comprising the amino acid sequence set forth in SEQ ID NO: 131, and a V_{L} comprising the amino acid sequence set forth in SEQ ID NO: 132. In certain embodiments, the extracellular antigen-binding domain is an scFv. In certain embodiments, the scFv is designated as “L12”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}—V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 17

| CDRs | 1 | 2 | 3 |
|---|---|---|---|
| V_{H} | GYTFTSY | NPSGGS | QLELLGNAFDI |
| | [SEQ ID NO: 21] | [SEQ ID NO: 2] | [SEQ ID NO: 56] |
| V_{L} | RASQSVSSSYLA | GASSRAT | QQYGNSPYT |
| | [SEQ ID NO: 57] | [SEQ ID NO: 58] | [SEQ ID NO: 130] |

| | |
|---|---|
| Full V_{H} | QVQLVQSGAEVKKPGASVKVSCKASGYTFTSYYMHWVRQAPGQGLEWMGIINPS |
| | GGSRSYAQKFQGRVTMTRDTSTSTVYMELSSLRSEDTAVYYCARQLELLGNAFD |
| | IWGQGTMVTVSS |
| | [SEQ ID NO: 131] |
| Full V_{L} | EIVLTQSPGTLSLSPGERATLSCRASQSVSSSYLAWYQQKPGQAPRLLIYGASS |
| | RATGIPDRFSGSGSGTDFTLTISRLEPEDFAVYYCQQYGNSPYTFGQGTKLEIK |
| | [SEQ ID NO: 132] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGTAGAAGCTACGCACAGAAGTTCCAGGGCAGAGTCACTATGACCAGG |
| | GACACGTCCACGAGCACAGTCTACATGGAGCTGAGCAGCCTGAGATCTGAGGAC |
| | ACGGCCGTGTATTACTGTGCGAGACAACTGGAACTTTTGGGGAATGCTTTTGAT |
| | ATCTGGGGCCAAGGGACAATGGTCACCGTCTCTTCA |
| | [SEQ ID NO: 133] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAACTCGCCGTACACTTTTGGCCAGGGGACCAAGCTGGAGATCAAA |
| | [SEQ ID NO: 134] |

In certain embodiments, the extracellular antigen-binding domain (e.g., an scFv) comprises a V_{H} comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof. SEQ ID NOs: 135-137 are provided in Table 17.

In certain embodiments, the extracellular antigen-binding domain (e.g., an scFv) comprises a V_{L} comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification thereof. SEQ ID NOs: 139-140 are provided in Table 17.

In certain embodiments, the extracellular antigen-binding domain (e.g., an scFv) comprises a V_{H} comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof; and a V_{L} comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification thereof.

In certain embodiments, the extracellular antigen-binding domain (e.g., an scFv) comprises a V_{H} comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a V_{L} comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 141. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 141. In certain embodiments, the extracellular antigen-binding domain comprises a V_{H} comprising the amino sequence set forth in SEQ ID NO: 141. An exemplary nucleotide sequence encoding the amino acid sequence of SEQ ID NO: 141 is set forth in SEQ ID NO: 143. SEQ ID NO: 141 and 143 are provided in Table 18 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 142. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 142. In certain embodiments, the extracellular antigen-binding domain comprises a V_{L} comprising the amino sequence set forth in SEQ ID NO: 142. An exemplary nucleotide sequence encoding the amino acid sequence of SEQ ID NO: 142 is set forth in SEQ ID NO: 144. SEQ ID NO: 142 and 144 are provided in Table 18 below.

In certain embodiments, the extracellular antigen-binding domain (e.g., an scFv) comprises a V_{H} comprising the amino acid sequence set forth in SEQ ID NO: 141, and a V_{L} comprising the amino acid sequence set forth in SEQ ID NO: 142. In certain embodiments, the extracellular antigen-binding domain is an scFv. In certain embodiments, the scFv is designated as “B22”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}—V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 18

| CDRs | 1 | 2 | 3 |
|---|---|---|---|
| V_{H} | GFTFNNF | KQDGSE | ESYFDL |
| | [SEQ ID NO: 135] | [SEQ ID NO: 136] | [SEQ ID NO: 137] |
| V_{L} | RSSTGAVTTSNYAN | GTNNRAP | ALWYSNRWV |
| | [SEQ ID NO: 138] | [SEQ ID NO: 139] | [SEQ ID NO: 140] |

| | |
|---|---|
| Full V_{H} | EVQLVESGGGLVQPGGSLRLSCAASGFTFNNFWMSWVRQAPGKGLEWVANIKQD |
| | GSEKYYVDSVKGRFTISRDNAKNSLYLQMNSLRAEDTAVYYCARESYFDLWGRG |
| | TLVTVSS |
| | [SEQ ID NO: 141] |
| Full V_{L} | QAVVTQESALTTSPGETVTLTCRSSTGAVTTSNYANWVQEKPDHLFTGLIGGTN |
| | NRAPGVPARFSGSLIGDKAALTITGAQTEDEAIYFCALWYSNRWVFGGGTKLTV |
| | L |
| | [SEQ ID NO: 142] |
| DNA for | GAGGTGCAGCTGGTGGAGTCTGGGGGAGGCTTGGTCCAGCCTGGGGGGTCCCTG |
| Full V_{H} | AGACTCTCCTGTGCAGCCTCTGGATTCACCTTTAATAACTTTTGGATGAGCTGG |
| | GTCCGCCAGGCTCCAGGGAAGGGGCTGGAGTGGGTGGCCAACATAAAGCAAGAT |
| | GGAAGTGAGAAATACTATGTGGACTCTGTGAAGGGCCGATTCACCATCTCCAGA |
| | GACAACGCCAAGAACTCACTGTATCTGCAAATGAACAGCCTGAGAGCCGAGGAC |
| | ACGGCTGTGTATTACTGTGCGAGAGAGAGTTACTTCGATCTCTGGGGCCGTGGC |
| | ACCCTGGTCACTGTCTCCTCA |
| | [SEQ ID NO: 143] |
| DNA for | CAGGCTGTTGTGACTCAGGAATCTGCACTCACCACATCACCTGGTGAAACAGTC |
| Full V_{L} | ACACTCACTTGTCGCTCAAGTACTGGGGCTGTTACAACTAGTAACTATGCCAAC |
| | TGGGTCCAAGAAAAACCAGATCATTTATTCACTGGTCTAATAGGTGGTACCAAC |
| | AACCGAGCTCCAGGTGTTCCTGCCAGATTCTCAGGCTCCCTGATTGGAGACAAG |
| | GCTGCCCTCACCATCACAGGGGCACAGACTGAGGATGAGGCAATATATTTCTGT |
| | GCTCTATGGTACAGCAACCGCTGGGTGTTCGGTGGAGGAACCAAACTGACTGTC |
| | CTA |
| | [SEQ ID NO: 144] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof. SEQ ID NOs: 21, 2, and 145 are provided in Table 19.

In certain embodiments, the extracellular antigen-binding domain (e.g., an scFv) comprises a V_{L} comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification thereof. SEQ ID NOs: 57, 146, and 125 are provided in Table 19.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof; and a V_{L} Comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a V_{L} comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 147. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 147. In certain embodiments, the extracellular antigen-binding domain comprises a V_{H} comprising the amino sequence set forth in SEQ ID NO: 147. An exemplary nucleotide sequence encoding the amino acid sequence of SEQ ID NO: 147 is set forth in SEQ ID NO: 149. SEQ ID NO: 147 and 149 are provided in Table 19 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 148. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 148. In certain embodiments, the extracellular antigen-binding domain comprises a V_{L} comprising the amino sequence set forth in SEQ ID NO: 148. An exemplary nucleotide sequence encoding the amino acid sequence of SEQ ID NO: 148 is set forth in SEQ ID NO: 150. SEQ ID NO: 148 and 150 are provided in Table 19 below.

In certain embodiments, the extracellular antigen-binding domain (e.g., an scFv) comprises a V_{H} comprising the amino acid sequence set forth in SEQ ID NO: 147, and a V_{L} comprising the amino acid sequence set forth in SEQ ID NO: 148. In certain embodiments, the extracellular antigen-binding domain is an scFv. In certain embodiments, the scFv is designated as “C22”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 19

| CDRs | 1 | 2 | 3 |
|---|---|---|---|
| V_{H} | GYTFTSY | NPSGGS | VADIVGATFDY |
| | [SEQ ID NO: 21] | [SEQ ID NO: 2] | [SEQ ID NO: 145] |
| V_{L} | RASQSVSSSYLA | GASSWAT | QQYDSSPYT |
| | [SEQ ID NO: 57] | [SEQ ID NO: 146] | [SEQ ID NO: 125] |

| | |
|---|---|
| Full V_{H} | QVQLVQSGAEVKKPGASVKVSCKASGYTFTSYYMHWVRQAPGQGLEWMGIINPS |
| | GGSTSYAQKFQGRVTMTRDTSTSTVYMELSSLRSEDTAVYYCARVADIVGATFD |
| | YWGQGTLVTVSS |
| | [SEQ ID NO: 147] |
| Full V_{L} | EIVLTQSPGTLSLSPGERATLSCRASQSVSSSYLAWYQQKPGQAPRLLIYGASS |
| | WATGIPDRFSGSGSGTDETLTISRLEPEDFAVYYCQQYDSSPYTFGQGTKLEIK |
| | [SEQ ID NO: 148] |
| DNA for | CAGGTGCAGCTGGTGCAGTCTGGGGCTGAGGTGAAGAAGCCTGGGGCCTCAGTG |
| Full V_{H} | AAAGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACTAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGAGTGGCGGACATAGTGGGAGCTACTTTTGAC |
| | TACTGGGGCCAGGGAACCCTGGTCACCGTCTCCTCA |
| | [SEQ ID NO: 149] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | TGGGCCACTGGCATCCCAGACAGGTTCAGTGGCAGTGGGTCTGGGACAGACTTC |
| | ACTCTCACCATCAGCAGACTGGAACCTGAAGATTTTGCAGTGTATTACTGTCAG |
| | CAGTATGATAGCTCACCCTACACTTTTGGCCAGGGGACCAAGCTGGAGATCAAA |
| | [SEQ ID NO: 150] |

In certain embodiments, the extracellular antigen-binding domain (e.g., an scFv) comprises a V_{H} comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof. SEQ ID NOs: 151, 2, and 152 are provided in Table 20.

In certain embodiments, the extracellular antigen-binding domain (e.g., an scFv) comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof. SEQ ID NOs: 57, 58, and 82 are provided in Table 20.

In certain embodiments, the extracellular antigen-binding domain (e.g., an scFv) comprises a V_{H} comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof; and a V_{L} Comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof.

In certain embodiments, the extracellular antigen-binding domain (e.g., an scFv) comprises a V_{H} comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 151. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 153. In certain embodiments, the extracellular antigen-binding domain comprises a V_{H} comprising the amino sequence set forth in SEQ ID NO: 153. An exemplary nucleotide sequence encoding the amino acid sequence of SEQ ID NO: 153 is set forth in SEQ ID NO: 155. SEQ ID NO: 153 and 155 are provided in Table 20 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 154. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 154. In certain embodiments, the extracellular antigen-binding domain comprises a V_{L} comprising the amino sequence set forth in SEQ ID NO: 154. An exemplary nucleotide sequence encoding the amino acid sequence of SEQ ID NO: 154 is set forth in SEQ ID NO: 156. SEQ ID NO: 154 and 156 are provided in Table 20 below.

In certain embodiments, the extracellular antigen-binding domain (e.g., an scFv) comprises a V_{H} comprising the amino acid sequence set forth in SEQ ID NO: 153, and a V_{L} comprising the amino acid sequence set forth in SEQ ID NO: 154. In certain embodiments, the extracellular antigen-binding domain is an scFv. In certain embodiments, the scFv is designated as “D8”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 20

| CDRs | 1 | 2 | 3 |
|---|---|---|---|
| V_{H} | GYTFTTY | NPSGGS | QGHYIGNYLDY |
| | [SEQ ID NO: 151] | [SEQ ID NO: 2] | [SEQ ID NO: 152] |
| V_{L} | RASQSVSSSYLA | GASSRAT | QQYGSSP |
| | [SEQ ID NO: 57] | [SEQ ID NO: 58] | [SEQ ID NO: 82] |

| | |
|---|---|
| Full V_{H} | QVQLVQSGAEVKKPGASVKVSCKASGYTFTTYYMHWVRQAPGQGLEWMGIINPS |
| | GGSTSYAQKFQGRVTMTRDTSTSTVYMELSSLRSEDTAVYYCARQGHYIGNYLD |
| | YWGQGTLVTVSS |
| | [SEQ ID NO: 153] |
| Full V_{L} | EIVLTQSPGTLSLSPGERATLSCRASQSVSSSYLAWYQQKPGQAPRLLIYGASS |
| | RATGIPDRFSGSGSGTDFTLTISRLEPEDFAVYYCQQYGSSPFGQGTKLEIK |
| | [SEQ ID NO: 154] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCACCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGACAGGGCCATTATATTGGGAACTACCTTGAC |
| | TACTGGGGCCAGGGAACCCTGGTCACCGTCTCCTCA |
| | [SEQ ID NO: 155] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTAGCTCACCTTTTGGCCAGGGGACCAAGCTGGAGATCAAA |
| | [SEQ ID NO: 156] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof. SEQ ID NOs: 21, 2, and 123 are provided in Table 21.

In certain embodiments, the extracellular antigen-binding domain (e.g., an scFv) comprises a V_{L} comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. SEQ ID NOs: 124, 58, and 59 are provided in Table 21.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof; and a V_{L} Comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a V_{L} comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 157. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 157. In certain embodiments, the extracellular antigen-binding domain comprises a V_{H} comprising the amino sequence set forth in SEQ ID NO: 157. An exemplary nucleotide sequence encoding the amino acid sequence of SEQ ID NO: 157 is set forth in SEQ ID NO: 159. SEQ ID NO: 157 and 159 are provided in Table 21 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 158. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 158. In certain embodiments, the extracellular antigen-binding domain comprises a V_{L} comprising the amino sequence set forth in SEQ ID NO: 158. An exemplary nucleotide sequence encoding the amino acid sequence of SEQ ID NO: 158 is set forth in SEQ ID NO: 160. SEQ ID NO: 158 and 160 are provided in Table 21 below.

In certain embodiments, the extracellular antigen-binding domain (e.g., an scFv) comprises a V_{H} comprising the amino acid sequence set forth in SEQ ID NO: 157, and a V_{L} comprising the amino acid sequence set forth in SEQ ID NO: 158. In certain embodiments, the extracellular antigen-binding domain is an scFv. In certain embodiments, the scFv is designated as “G16”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 21

| CDRs | 1 | 2 | 3 |
|---|---|---|---|
| V_{H} | GYTFTSY | NPSGGS | QGGAQWLVLAFDI |
| | [SEQ ID NO: 21] | [SEQ ID NO: 2] | [SEQ ID NO: 123] |
| V_{L} | RASQSVSSYLA | GASSRAT | QQYGSSPYT |
| | [SEQ ID NO: 124] | [SEQ ID NO: 58] | [SEQ ID NO: 59] |

| | |
|---|---|
| Full V_{H} | QVQLVQSGAEVKKPGASVQVSCKASGYTFTSYYMHWVRQAPGGLEWMGIINPS |
| | GGSTSYAQKFQGRVTMTRDTSTSTVYMELSSLRSEDTAVYYCARQGGAQWLVLA |
| | FDIWGQGTMVTVSS |
| | [SEQ ID NO: 157] |
| Full V_{L} | EIVLTQSPGTLSLSPGERATLSCRASQSVSSYLAWYQQKPGQAPRLLIYGASSR |
| | ATGIPDRESGGGSGTDETLTISRLEPEDFAVYYCQQYGSSPYTFGQGTKLEIK |
| | [SEQ ID NO: 158] |
| DNA for | CAGGTGCAGCTGGTGCAGTCTGGGGCTGAGGTGAAGAAGCCTGGGGCCTCAGTG |
| Full V_{H} | CAGGTTTCCTGCAAGGCATCTGGATACACCTTCACCAGCTACTATATGCACTGG |
| | GTGCGACAGGCCCCTGGACAAGGGCTTGAGTGGATGGGAATAATCAACCCTAGT |
| | GGTGGTAGCACAAGCTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGACAGGGGGGAGCCCAGTGGCTGGTACTTGCT |
| | TTTGATATCTGGGGCCAAGGGACAATGGTCACCGTCTCTTCA |
| | [SEQ ID NO: 159] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCTACTTAGCCTGGTAC |
| | CAGCAGAAACCTGGCCAGGCTCCCAGGCTCCTCATCTATGGTGCATCCAGCAGG |
| | GCCACTGGCATCCCAGACAGGTTCAGTGGCGGTGGGTCTGGGACAGACTTCACT |
| | CTCACCATCAGCAGACTGGAGCCTGAAGATTTTGCAGTGTATTACTGTCAGCAG |
| | TATGGTAGCTCACCGTACACTTTTGGCCAGGGGACCAAGCTGGAGATCAAA |
| | [SEQ ID NO: 160] |

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof. SEQ ID NOs: 11, 136, and 161 are provided in Table 22.

In certain embodiments, the extracellular antigen-binding domain (e.g., an scFv) comprises a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification thereof. SEQ ID NOs: 73, 74, and 162 are provided in Table 22.

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof; and a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification thereof.

In certain embodiments, the extracellular antigen-binding domain (e.g., an scFv) comprises a V_{H} comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a V_{L} Comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 163. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 163. In certain embodiments, the extracellular antigen-binding domain comprises a V_{H} comprising the amino sequence set forth in SEQ ID NO: 163. An exemplary nucleotide sequence encoding the amino acid sequence of SEQ ID NO: 163 is set forth in SEQ ID NO: 165. SEQ ID NO: 163 and 165 are provided in Table 22 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 164. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 148. In certain embodiments, the extracellular antigen-binding domain comprises a V_{L} comprising the amino sequence set forth in SEQ ID NO: 164. An exemplary nucleotide sequence encoding the amino acid sequence of SEQ ID NO: 164 is set forth in SEQ ID NO: 166. SEQ ID NO: 164 and 166 are provided in Table 22 below.

In certain embodiments, the extracellular antigen-binding domain (e.g., an scFv) comprises a V_{H} comprising the amino acid sequence set forth in SEQ ID NO: 163, and a V_{L} comprising the amino acid sequence set forth in SEQ ID NO: 164. In certain embodiments, the extracellular antigen-binding domain is an scFv. In certain embodiments, the scFv is designated as “F21”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 22

| CDRs | 1 | 2 | 3 |
|---|---|---|---|
| V_{H} | GFTFSSY | KQDGSE | EWLRFGGLVY |
| | [SEQ ID NO: 11] | [SEQ ID NO: 136] | [SEQ ID NO: 161] |
| V_{L} | RASQSVSSNLA | GASTRAT | QQYNNWPLT |
| | [SEQ ID NO: 73] | [SEQ ID NO: 74] | [SEQ ID NO: 162] |

| | |
|---|---|
| Full V_{H} | EVQLVESGGGLVQPGGSLRLSCAASGFTFSSYWMSWVRQAPGKGLEWVANIKQD |
| | GSEKYYVDSVKGRFTISRDNAKNSLYLQMNSLRAEDTAVYYCAREWLRFGGLVY |
| | WGQGTLVTVSS |
| | [SEQ ID NO: 163] |
| Full V_{L} | EIVMTQSPATLSVSPGERATLSCRASQSVSSNLAWYQQKPGQAPRLLIYGASTR |
| | ATGIPARFSGSGSGTEFTLTISSLQSEDFAVYYCQQYNNWPLTFGGGTKVEIK |
| | [SEQ ID NO: 164] |
| DNA for | GAGGTGCAGCTGGTGGAGTCTGGGGGAGGCTTGGTCCAGCCTGGGGGGTCCCTG |
| Full V_{H} | AGACTCTCCTGTGCAGCCTCTGGATTCACCTTTAGTAGCTATTGGATGAGCTGG |
| | GTCCGCCAGGCTCCAGGGAAGGGGCTGGAGTGGGTGGCCAACATAAAGCAAGAT |
| | GGAAGTGAGAAATACTATGTGGACTCTGTGAAGGGCCGATTCACCATCTCCAGA |
| | GACAACGCCAAGAACTCACTGTATCTGCAAATGAACAGCCTGAGAGCCGAGGAC |
| | ACGGCTGTGTATTACTGTGCGAGAGAGTGGCTACGATTTGGGGGCTTAGTGTAC |
| | TGGGGCCAGGGAACCCTGGTCACCGTCTCCTCA |
| | [SEQ ID NO: 165] |
| DNA for | GAAATAGTGATGACGCAGTCTCCAGCCACCCTGTCTGTGTCTCCAGGGGAAAGA |
| Full V_{L} | GCCACCCTCTCCTGCAGGGCCAGTCAGAGTGTTAGCAGCAACTTAGCCTGGTAC |
| | CAGCAGAAACCTGGCCAGGCTCCCAGGCTCCTCATCTATGGTGCATCCACCAGG |
| | GCCACTGGTATCCCAGCCAGGTTCAGTGGCAGTGGGTCTGGGACAGAGTTCACT |
| | CTCACCATCAGCAGCCTGCAGTCTGAAGATTTTGCAGTTTATTACTGTCAGCAG |
| | TATAATAACTGGCCGCTCACTTTCGGCGGAGGGACCAAGGTGGAGATCAAA |
| | [SEQ ID NO: 166] |

In certain embodiments, the extracellular antigen-binding domain (e.g., an scFv) comprises a V_{H} comprising a CDR1 comprising the amino acid sequence set forth in SEQ ID NO: 96 or a conservative modification thereof, a CDR2 comprising the amino acid sequence set forth in SEQ ID NO: 167 or a conservative modification thereof, and a CDR3 comprising the amino acid sequence set forth in SEQ ID NO: 168 or a conservative modification thereof. SEQ ID NOs: 96, 167, and 168 are provided in Table 23.

In certain embodiments, the extracellular antigen-binding domain (e.g., an scFv) comprises a V_{L} comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification thereof. SEQ ID NOs: 169-171 are provided in Table 23.

In certain embodiments, the extracellular antigen-binding domain (e.g., an scFv) comprises a V_{H} comprising a CDR1 comprising the amino acid sequence set forth in SEQ ID NO: 96 or a conservative modification thereof, a CDR2 comprising the amino acid sequence set forth in SEQ ID NO: 167 or a conservative modification thereof, and a CDR3 comprising the amino acid sequence set forth in SEQ ID NO: 168 or a conservative modification thereof; and a V_{L} comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification thereof.

In certain embodiments, the extracellular antigen-binding domain (e.g., an scFv) comprises a V_{H} comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a V_{L} Comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 172. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 172. In certain embodiments, the extracellular antigen-binding domain comprises a V_{H} comprising the amino sequence set forth in SEQ ID NO: 172. An exemplary nucleotide sequence encoding the amino acid sequence of SEQ ID NO: 172 is set forth in SEQ ID NO: 174. SEQ ID NO: 172 and 174 are provided in Table 23 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 173. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 173. In certain embodiments, the extracellular antigen-binding domain comprises a V_{L} comprising the amino sequence set forth in SEQ ID NO: 173. An exemplary nucleotide sequence encoding the amino acid sequence of SEQ ID NO: 173 is set forth in SEQ ID NO: 175. SEQ ID NO: 173 and 175 are provided in Table 23 below.

In certain embodiments, the extracellular antigen-binding domain (e.g., an scFv) comprises a V_{H} comprising the amino acid sequence set forth in SEQ ID NO: 172, and a V_{L} comprising the amino acid sequence set forth in SEQ ID NO: 173. In certain embodiments, the extracellular antigen-binding domain is an scFv. In certain embodiments, the scFv is designated as “N12”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 23

| CDRs | 1 | 2 | 3 |
|---|---|---|---|
| V_{H} | GYTFTGY | NPNSGG | DGVITSFDY |
| | [SEQ ID NO: 96] | [SEQ ID NO: 167] | [SEQ ID NO: 168] |
| V_{L} | KSSQSVLYSSNIKTYLA | WASTRES | QQYYSAPYT |
| | [SEQ ID NO: 169] | [SEQ ID NO: 170] | [SEQ ID NO: 171] |

| | |
|---|---|
| Full V_{H} | QVQLVQSGAEVKKPGASVKVSCKASGYTFTGYYMHWVRQAPGQGLEWMGWINPN |
| | SGGTNYAQKFQGRVTMTRDTSISTAYMELSRLRSDDTAVYYCARDGVITSFDYW |
| | GQGTLVTVSS |
| | [SEQ ID NO: 172] |
| Full V_{L} | DIVMTQSPDSLAVSLGERATINCKSSQSVLYSSNIKTYLAWYQQKPGQPPKLLI |
| | YWASTRESGVPDRFSGGGSGTDFTLTITSLQAEDVAVYYCQQYYSAPYTFGQGT |
| | KLEIK |
| | [SEQ ID NO: 173] |
| DNA for | CAGGTGCAGCTGGTGCAGTCTGGGGCTGAGGTGAAGAAGCCTGGGGCCTCAGTG |
| Full V_{H} | AAGGTCTCCTGCAAGGCTTCTGGATACACTTTCACCGGCTACTATATGCACTGG |
| | GTGCGACAGGCCCCTGGACAAGGGCTTGAGTGGATGGGATGGATCAACCCTAAC |
| | AGTGGTGGCACAAACTATGCACAGAAGTTTCAGGGCAGGGTCACCATGACCAGG |
| | GACACGTCCATCAGCACAGCCTACATGGAGCTGAGCAGGCTGAGATCTGACGAC |
| | ACGGCCGTGTATTACTGTGCGAGAGACGGGGTGATTACGTCTTTTGACTACTGG |
| | GGCCAGGGAACCCTGGTCACCGTCTCCTCA |
| | [SEQ ID NO: 174] |
| DNA for | GACATCGTGATGACCCAGTCTCCAGACTCCCTGGCTGTGTCTCTGGGCGAGAGG |
| Full V_{L} | GCCACCATCAACTGCAAGTCCAGCCAGAGTGTTTTATACAGCTCCAACATTAAG |
| | ACCTACTTAGCTTGGTACCAGCAGAAACCAGGACAGCCTCCTAAGCTGCTCATT |
| | TACTGGGCATCTACCCGGGAATCCGGAGTCCCTGACCGATTCAGTGGCGGCGGG |
| | TCTGGGACAGATTTCACTCTCACCATCACCAGCCTGCAGGCTGAAGATGTGGCA |
| | GTTTATTACTGTCAGCAGTATTATAGTGCTCCGTACACTTTTGGCCAGGGGACC |
| | AAGCTGGAGATCAAA |
| | [SEQ ID NO: 175] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 176 or a conservative modification thereof, and a CDR3 comprising the amino acid sequence set forth in SEQ ID NO: 177 or a conservative modification thereof. SEQ ID NOs: 21, 176, and 177 are provided in Table 24.

In certain embodiments, the extracellular antigen-binding domain (e.g., an scFv) comprises a V_{L} comprising a CDR1 comprising the amino acid sequence set forth in SEQ ID NO: 178 or a conservative modification thereof, a CDR2 comprising the amino acid sequence set forth in SEQ ID NO: 50 or a conservative modification thereof, and a CDR3 comprising the amino acid sequence set forth in SEQ ID NO: 179 or a conservative modification thereof. SEQ ID NOs: 178, 50, and 179 are provided in Table 24.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 176 or a conservative modification thereof, and a CDR3 comprising the amino acid sequence set forth in SEQ ID NO: 177 or a conservative modification thereof and a V_{L} comprising a CDR1 comprising the amino acid sequence set forth in SEQ ID NO: 178 or a conservative modification thereof, a CDR2 comprising the amino acid sequence set forth in SEQ ID NO: 50 or a conservative modification thereof, and a CDR3 comprising the amino acid sequence set forth in SEQ ID NO: 179 or a conservative modification thereof.

In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177; and a V_{L} Comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 179.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 180. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 180. In certain embodiments, the extracellular antigen-binding domain comprises a V_{H} comprising the amino sequence set forth in SEQ ID NO: 180. An exemplary nucleotide sequence encoding the amino acid sequence of SEQ ID NO: 180 is set forth in SEQ ID NO: 182. SEQ ID NO: 180 and 182 are provided in Table 24 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 181. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 181. In certain embodiments, the extracellular antigen-binding domain comprises a V_{L} comprising the amino sequence set forth in SEQ ID NO: 181. An exemplary nucleotide sequence encoding the amino acid sequence of SEQ ID NO: 181 is set forth in SEQ ID NO: 183. SEQ ID NO: 181 and 183 are provided in Table 24 below.

In certain embodiments, the extracellular antigen-binding domain (e.g., an scFv) comprises a V_{H} comprising the amino acid sequence set forth in SEQ ID NO: 180, and a V_{L} comprising the amino acid sequence set forth in SEQ ID NO: 181. In certain embodiments, the extracellular antigen-binding domain is an scFv. In certain embodiments, the scFv is designated as “G23”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 24

| CDRs | 1 | 2 | 3 |
|---|---|---|---|
| V_{H} | GYTFTSY | DPSDGS | DREYNYYGLDV |
| | [SEQ ID NO: 21] | [SEQ ID NO: 176] | [SEQ ID NO: 177] |
| V_{L} | RSSQSLVYRDGNTYLN | KVSNRDS | MQGTHWPPT |
| | [SEQ ID NO: 178] | [SEQ ID NO: 50] | [SEQ ID NO: 179] |

| | |
|---|---|
| Full V_{H} | QVQLVQSGAEVKKPGASVKVSCKASGYTFTSYYIHWVRQAPGQGLEWMGIIDPS |
| | DGSTNYAQKFQGRVTMTRDTSTSTVYMELSSLRSEDTAVYYCARDREYNYYGLD |
| | VWGQGTTVTVSS |
| | [SEQ ID NO: 180] |
| Full V_{L} | DVVMTQSPLSLPVTLGQPASISCRSSQSLVYRDGNTYLNWFQQRPGQSPRRLIY |
| | KVSNRDSGVPDRFRGSGSGTDFTLKISRVEAEDVGVYYCMQGTHWPPTFGQGTK |
| | VEIK |
| | [SEQ ID NO: 181] |
| DNA for | CAGGTGCAGCTGGTGCAGTCTGGGGCTGAGGTGAAGAAGCCTGGGGCCTCAGTG |
| Full V_{H} | AAGGTTTCCTGCAAGGCATCTGGATACACCTTCACCAGCTACTATATACACTGG |
| | GTGCGACAGGCCCCTGGACAAGGGCTTGAGTGGATGGGAATAATCGACCCAAGT |
| | GATGGTAGCACAAACTACGCACAGAAGTTCCAGGGCAGAGTCACCATGACCAGG |
| | GACACGTCCACGAGCACAGTCTACATGGAGCTGAGCAGCCTGAGATCTGAGGAC |
| | ACGGCCGTGTATTACTGTGCGAGAGATCGGGAATATAACTACTACGGTTTGGAC |
| | GTCTGGGGCCAAGGGACCACGGTCACCGTCTCCTCA |
| | [SEQ ID NO: 182] |
| DNA for | GATGTTGTGATGACTCAGTCTCCACTCTCCCTGCCCGTCACCCTTGGACAGCCG |
| Full V_{L} | GCCTCCATCTCCTGCAGGTCTAGTCAAAGCCTCGTATACCGTGATGGAAACACC |
| | TACTTGAATTGGTTTCAGCAGAGGCCAGGCCAATCTCCAAGGCGCCTAATTTAT |
| | AAGGTTTCTAACCGGGACTCTGGGGTCCCAGACAGATTCCGCGGCAGTGGGTCA |
| | GGCACTGATTTCACACTGAAAATCAGCCGGGTGGAGGCTGAGGATGTTGGGGTT |
| | TATTACTGCATGCAAGGTACACACTGGCCTCCGACGTTCGGCCAAGGGACCAAG |
| | GTGGAAATCAAA |
| | [SEQ ID NO: 183] |

In certain embodiments, the extracellular antigen-binding domain (e.g., an scFv) comprises a V_{H} comprising a CDR1 comprising the amino acid sequence set forth in SEQ ID NO: 184 or a conservative modification thereof, a CDR2 comprising the amino acid sequence set forth in SEQ ID NO: 185 or a conservative modification thereof, and a CDR3 comprising the amino acid sequence set forth in SEQ ID NO: 186 or a conservative modification thereof. SEQ ID NOs: 184-186 are provided in Table 25.

In certain embodiments, the extracellular antigen-binding domain (e.g., an scFv) comprises a V_{L} comprising a CDR1 comprising the amino acid sequence set forth in SEQ ID NO: 187 or a conservative modification thereof, a CDR2 comprising the amino acid sequence set forth in SEQ ID NO: 188 or a conservative modification thereof, and a CDR3 comprising the amino acid sequence set forth in SEQ ID NO: 189 or a conservative modification thereof. SEQ ID NOs: 187-189 are provided in Table 25.

In certain embodiments, the extracellular antigen-binding domain (e.g., an scFv) comprises a V_{H} comprising a CDR1 comprising the amino acid sequence set forth in SEQ ID NO: 184 or a conservative modification thereof, a CDR2 comprising the amino acid sequence set forth in SEQ ID NO: 185 or a conservative modification thereof, and a CDR3 comprising the amino acid sequence set forth in SEQ ID NO: 186 or a conservative modification thereof, a V_{L} Comprising a CDR1 comprising the amino acid sequence set forth in SEQ ID NO: 187 or a conservative modification thereof, a CDR2 comprising the amino acid sequence set forth in SEQ ID NO: 188 or a conservative modification thereof, and a CDR3 comprising the amino acid sequence set forth in SEQ ID NO: 189 or a conservative modification thereof.

In certain embodiments, the extracellular antigen-binding domain (e.g., an scFv) comprises a V_{H} comprising a CDR1 comprising the amino acid sequence set forth in SEQ ID NO: 184, a CDR2 comprising the amino acid sequence set forth in SEQ ID NO: 185, and a CDR3 comprising the amino acid sequence set forth in SEQ ID NO: 186; and a V_{L} comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 190. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 190. In certain embodiments, the extracellular antigen-binding domain comprises a V_{H} comprising the amino sequence set forth in SEQ ID NO: 190. An exemplary nucleotide sequence encoding the amino acid sequence of SEQ ID NO: 190 is set forth in SEQ ID NO: 192. SEQ ID NO: 190 and 192 are provided in Table 25 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 191. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 191. In certain embodiments, the extracellular antigen-binding domain comprises a V_{L} comprising the amino sequence set forth in SEQ ID NO: 191. An exemplary nucleotide sequence encoding the amino acid sequence of SEQ ID NO: 191 is set forth in SEQ ID NO: 193. SEQ ID NO: 191 and 193 are provided in Table 25 below.

In certain embodiments, the extracellular antigen-binding domain (e.g., an scFv) comprises a V_{H} comprising the amino acid sequence set forth in SEQ ID NO: 190, and a V_{L} comprising the amino acid sequence set forth in SEQ ID NO: 191. In certain embodiments, the extracellular antigen-binding domain is an scFv. In certain embodiments, the scFv is designated as “I1”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 25

| CDRs | 1 | 2 | 3 |
|---|---|---|---|
| V_{H} | GFTFSNTW | IKSKSDGGTT | TQYYWNSFDY |
| | [SEQ ID NO: 184] | [SEQ ID NO: 185] | [SEQ ID NO: 186] |
| V_{L} | QASQDISNYLN | DASNLET | QQYDNLPLT |
| | [SEQ ID NO: 187] | [SEQ ID NO: 188] | [SEQ ID NO: 189] |

| | |
|---|---|
| Full V_{H} | EVQLVESGGGLVKPGGSLRLSCAASGFTFSNTWMSWVRQAPGKGLEWVGRIKSK |
| | SDGGTTDYAAPVKGRFTISRDDSKNTLYLQMNSLKTEDTAVYYCTQYYWNSFDY |
| | WGQGTLVTVSS |
| | [SEQ ID NO: 190] |
| Full V_{L} | DIQMTQSPSSLSASVGDRVTITCQASQDISNYLNWYQQKPGKAPKLLIYDASNL |
| | ETGVPSRFSGSGSGTDFTFTISSLQPEDIATYYCQQYDNLPLTFGGGTKVEIK |
| | [SEQ ID NO: 191] |
| DNA for | GAGGTGCAGCTGGTGGAGTCTGGGGGGGGCTTGGTAAAGCCTGGGGGGTCCCTT |
| Full V_{H} | AGACTCTCCTGTGCAGCCTCTGGATTCACTTTCAGTAACACCTGGATGAGCTGG |
| | GTCCGCCAGGCTCCAGGGAAGGGGCTGGAGTGGGTTGGCCGTATTAAAAGCAAA |
| | TCTGATGGTGGGACAACAGACTACGCTGCACCCGTGAAAGGCAGATTCACCATC |
| | TCAAGAGATGATTCAAAAAACACGCTGTATCTGCAAATGAACAGCCTGAAAACC |
| | GAGGACACAGCCGTGTATTACTGTACCCAGTATTATTGGAACTCCTTTGACTAC |
| | TGGGGCCAGGGAACCCTGGTCACCGTCTCCTCA |
| | [SEQ ID NO: 192] |
| DNA for | GACATCCAGATGACCCAGTCTCCATCCTCCCTGTCTGCATCTGTAGGAGACAGA |
| Full V_{L} | GTCACCATCACTTGCCAGGCGAGTCAGGACATTAGCAACTATTTAAATTGGTAT |
| | CAGCAGAAACCAGGGAAAGCCCCTAAGCTCCTGATCTACGATGCATCCAATTTG |
| | GAAACAGGGGTCCCATCAAGGTTCAGTGGAAGTGGATCTGGGACAGATTTTACT |
| | TTCACCATCAGCAGCCTGCAGCCTGAAGATATTGCAACATATTACTGTCAACAG |
| | TATGATAATCTCCCGCTCACTTTCGGCGGAGGGACCAAGGTGGAGATCAAA |
| | [SEQ ID NO: 193] |

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 194 or a conservative modification thereof, and a CDR3 comprising the amino acid sequence set forth in SEQ ID NO: 195 or a conservative modification thereof. SEQ ID NOs: 11, 194, and 195 are provided in Table 26.

In certain embodiments, the extracellular antigen-binding domain (e.g., an scFv) comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 196 or a conservative modification thereof. SEQ ID NOs: 4, 5 and 196 are provided in Table 26.

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 194 or a conservative modification thereof, and a CDR3 comprising the amino acid sequence set forth in SEQ ID NO: 195 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 196 or a conservative modification thereof.

In certain embodiments, the extracellular antigen-binding domain (e.g., an scFv) comprises a V_{H} comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a V_{L} Comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 196.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 197. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 197. In certain embodiments, the extracellular antigen-binding domain comprises a V_{H} comprising the amino sequence set forth in SEQ ID NO: 197. An exemplary nucleotide sequence encoding the amino acid sequence of SEQ ID NO: 197 is set forth in SEQ ID NO: 199. SEQ ID NO: 197 and 199 are provided in Table 26 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 198. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 198. In certain embodiments, the extracellular antigen-binding domain comprises a V_{L} comprising the amino sequence set forth in SEQ ID NO: 198. An exemplary nucleotide sequence encoding the amino acid sequence of SEQ ID NO: 198 is set forth in SEQ ID NO: 200. SEQ ID NO: 198 and 200 are provided in Table 26 below.

In certain embodiments, the extracellular antigen-binding domain (e.g., an scFv) comprises a V_{H} comprising the amino acid sequence set forth in SEQ ID NO: 197, and a V_{L} comprising the amino acid sequence set forth in SEQ ID NO: 198. In certain embodiments, the extracellular antigen-binding domain is an scFv. In certain embodiments, the scFv is designated as “C8”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 26

| CDRs | 1 | 2 | 3 |
|---|---|---|---|
| V_{H} | GFTFSSY | KEDGSE | DPGWAPFDY |
| | [SEQ ID NO: 11] | [SEQ ID NO: 194] | [SEQ ID NO: 195] |
| V_{L} | RASQGISNYLA | AASSLQS | QQYNSFPYT |
| | [SEQ ID NO: 4] | [SEQ ID NO: 5] | [SEQ ID NO: 196] |

| | |
|---|---|
| Full V_{H} | EVQLVESGGGLVQPGGSQRLSCAASGFTFSSYWMNWVRQAPGKGLEWVANIKED |
| | GSEKYYVDSVKGRFTISRDNAKNSLYLQMNSLRAEDTAVYYCARDPGWAPFDYW |
| | GQGTLVTVSS |
| | [SEQ ID NO: 197] |
| Full V_{L} | DIQMSQSPSSLSASVGDRVTITCRASQGISNYLAWFQQKPGKAPKSLIYAASSL |
| | QSGVPSKFSGSGSGTDFTLAISSLQPEDFATYYCQQYNSFPYTFGQGTTLEIK |
| | [SEQ ID NO: 198] |
| DNA for | GAGGTGCAGCTGGTGGAGTCTGGGGGAGGCTTGGTCCAGCCTGGGGGGTCCCAG |
| Full V_{H} | AGACTCTCCTGTGCAGCCTCTGGATTCACCTTTAGTAGCTATTGGATGAACTGG |
| | GTCCGCCAGGCTCCAGGGAAGGGGCTGGAGTGGGTGGCCAACATAAAGGAAGAT |
| | GGAAGTGAGAAATACTATGTGGACTCTGTGAAGGGCCGATTCACCATCTCCAGA |
| | GACAACGCCAAGAACTCACTGTATCTGCAAATGAACAGCCTGAGAGCCGAGGAC |
| | ACGGCTGTGTATTACTGTGCGAGAGATCCGGGCTGGGCTCCCTTTGACTACTGG |
| | GGCCAGGGAACCCTGGTCACCGTCTCCTCA |
| | [SEQ ID NO: 199] |
| DNA for | GACATCCAGATGTCCCAGTCTCCATCCTCACTGTCTGCATCTGTAGGAGACAGA |
| Full V_{L} | GTCACCATCACTTGTCGGGCGAGTCAGGGCATTAGCAATTATTTAGCCTGGTTT |
| | CAGCAGAAACCAGGGAAAGCCCCTAAGTCCCTGATCTATGCTGCATCCAGTTTG |
| | CAAAGTGGGGTCCCATCAAAGTTCAGCGGCAGTGGATCTGGGACAGATTTCACT |
| | CTCGCCATCAGCAGCCTGCAGCCTGAAGATTTTGCAACTTATTACTGCCAACAG |
| | TATAATAGTTTCCCGTACACTTTTGGCCAGGGGACCACGCTGGAGATCAAA |
| | [SEQ ID NO: 200] |

In certain embodiments, the extracellular antigen-binding domain (e.g., an scFv) comprises a V_{H} comprising a CDR1 comprising the amino acid sequence set forth in SEQ ID NO: 201 or a conservative modification thereof, a CDR2 comprising the amino acid sequence set forth in SEQ ID NO: 202 or a conservative modification thereof, and a CDR3 comprising the amino acid sequence set forth in SEQ ID NO: 203 or a conservative modification thereof. SEQ ID NOs: 201-203 are provided in Table 27.

In certain embodiments, the extracellular antigen-binding domain (e.g., an scFv) comprises a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 204 or a conservative modification thereof. SEQ ID NOs: 57, 58, and 204 are provided in Table 27.

In certain embodiments, the extracellular antigen-binding domain (e.g., an scFv) comprises a V_{H} comprising a CDR1 comprising the amino acid sequence set forth in SEQ ID NO: 201 or a conservative modification thereof, a CDR2 comprising the amino acid sequence set forth in SEQ ID NO: 202 or a conservative modification thereof, and a CDR3 comprising the amino acid sequence set forth in SEQ ID NO: 203 or a conservative modification thereof; and a V_{L} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 204 or a conservative modification thereof.

In certain embodiments, the extracellular antigen-binding domain (e.g., an scFv) comprises a V_{H} comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203; and a V_{L} comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 205. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 205. In certain embodiments, the extracellular antigen-binding domain comprises a V_{H} comprising the amino sequence set forth in SEQ ID NO: 205. An exemplary nucleotide sequence encoding the amino acid sequence of SEQ ID NO: 205 is set forth in SEQ ID NO: 207. SEQ ID NO: 205 and 207 are provided in Table 27 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 206. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 206. In certain embodiments, the extracellular antigen-binding domain comprises a V_{L} comprising the amino sequence set forth in SEQ ID NO: 206. An exemplary nucleotide sequence encoding the amino acid sequence of SEQ ID NO: 206 is set forth in SEQ ID NO: 208. SEQ ID NO: 206 and 208 are provided in Table 27 below.

In certain embodiments, the extracellular antigen-binding domain (e.g., an scFv) comprises a V_{H} comprising the amino acid sequence set forth in SEQ ID NO: 205, and a V_{L} comprising the amino acid sequence set forth in SEQ ID NO: 206. In certain embodiments, the extracellular antigen-binding domain is an scFv. In certain embodiments, the scFv is designated as “018”. In certain embodiments, the V_{H} and V_{L} are linked via a linker. In certain embodiments, the linker comprises the amino acid sequence set forth in SEQ ID NO: 210.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 27

| CDRs | 1 | 2 | 3 |
|---|---|---|---|
| V_{H} | GGSINSY | FYSGI | IGVAGFYFDY |
| | [SEQ ID NO: 201] | [SEQ ID NO: 202] | [SEQ ID NO: 203] |
| V_{L} | RASQSVSSSYLA | GASSRAT | QQYGTSPLT |
| | [SEQ ID NO: 57] | [SEQ ID NO: 58] | [SEQ ID NO: 204] |

| | |
|---|---|
| Full V_{H} | QVQLQESGPGLVKPSETLSLTCTVSGGSINSYYWSWIRQPPGKGLEWIGYIFYS |
| | GITNYNPSLKSRVTISLDTSKNQFSLKLSSVTAADTAVYYCARIGVAGFYFDYW |
| | GQGTLVTVSS |
| | [SEQ ID NO: 205] |
| Full V_{L} | EIVLTQSPGTLSLSPGERATLSCRASQSVSSSYLAWYQQKPGQAPRLLIYGASS |
| | RATGIPDRFSGSGSGTDETLTISRLEPEDFAVYYCQQYGTSPLTFGGGTKVEIK |
| | [SEQ ID NO: 206] |
| DNA for | CAGGTGCAGCTGCAGGAGTCGGGCCCAGGACTGGTGAAGCCTTCGGAGACCCTG |
| Full V_{H} | TCCCTCACCTGCACTGTCTCTGGTGGCTCCATCAATAGTTACTACTGGAGCTGG |
| | ATCCGGCAGCCCCCAGGGAAGGGACTGGAGTGGATTGGGTATATCTTTTACAGT |
| | GGGATCACCAACTACAACCCCTCCCTCAAGAGTCGAGTCACCATATCATTAGAC |
| | ACGTCCAAGAACCAGTTCTCCCTGAAGCTGAGCTCTGTGACCGCTGCGGACACG |
| | GCCGTGTATTACTGTGCGAGAATCGGCGTGGCTGGTTTTTACTTTGACTACTGG |
| | GGCCAGGGAACCCTGGTCACCGTCTCCTCA |
| | [SEQ ID NO: 207] |
| DNA for | GAAATTGTGTTGACGCAGTCTCCAGGCACCCTGTCTTTGTCTCCAGGGGAAAGA |
| Full V_{L} | GCCACCCTCTCCTGCAGGGCCAGTCAGAGTGTTAGCAGCAGCTACTTAGCCTGG |
| | TACCAGCAGAAACCTGGCCAGGCTCCCAGGCTCCTCATCTATGGTGCATCCAGC |
| | AGGGCCACTGGCATCCCAGACAGGTTCAGTGGCAGTGGGTCTGGGACAGACTTC |
| | ACTCTCACCATCAGCAGACTGGAGCCTGAAGATTTTGCAGTGTATTACTGTCAG |
| | CAGTATGGTACCTCACCGCTCACTTTCGGCGGAGGGACCAAGGTGGAGATCAAA |
| | [SEQ ID NO: 208] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 7, SEQ ID NO: 8, SEQ ID NO: 17, SEQ ID NO: 18, SEQ ID NO: 24, SEQ ID NO: 25, SEQ ID NO: 34, SEQ ID NO: 35, SEQ ID NO: 42, SEQ ID NO: 43, SEQ ID NO: 52, SEQ ID NO: 53, SEQ ID NO: 60, SEQ ID NO: 61, SEQ ID NO: 66, SEQ ID NO: 67, SEQ ID NO: 76, SEQ ID NO: 77, SEQ ID NO: 83, SEQ ID NO: 84, SEQ ID NO: 92, SEQ ID NO: 93, SEQ ID NO: 102, SEQ ID NO: 103, SEQ ID NO: 108, SEQ ID NO: 109, SEQ ID NO: 113, SEQ ID NO: 119, SEQ ID NO: 120, SEQ ID NO: 126, SEQ ID NO: 127, SEQ ID NO: 131, SEQ ID NO: 132, SEQ ID NO: 141, SEQ ID NO: 142, SEQ ID NO: 147, SEQ ID NO: 148, SEQ ID NO: 153, SEQ ID NO: 154, SEQ ID NO: 157, SEQ ID NO: 158, SEQ ID NO: 163, SEQ ID NO: 164, SEQ ID NO: 172, SEQ ID NO: 173, SEQ ID NO: 180, SEQ ID NO: 181, SEQ ID NO: 190, SEQ ID NO: 191, SEQ ID NO: 197, SEQ ID NO: 198, SEQ ID NO: 205, or SEQ ID NO: 206) may contain substitutions (e.g., conservative substitutions), insertions, or deletions relative to the specified sequence(s), but retain the ability to bind to DLL3.

In certain embodiments, a total of 1 to 10 amino acids are substituted, inserted and/or deleted in a specific sequence (e.g., SEQ ID NO: 7, SEQ ID NO: 8, SEQ ID NO: 17, SEQ ID NO: 18, SEQ ID NO: 24, SEQ ID NO: 25, SEQ ID NO: 34, SEQ ID NO: 35, SEQ ID NO: 42, SEQ ID NO: 43, SEQ ID NO: 52, SEQ ID NO: 53, SEQ ID NO: 60, SEQ ID NO: 61, SEQ ID NO: 66, SEQ ID NO: 67, SEQ ID NO: 76, SEQ ID NO: 77, SEQ ID NO: 83, SEQ ID NO: 84, SEQ ID NO: 92, SEQ ID NO: 93, SEQ ID NO: 102, SEQ ID NO: 103, SEQ ID NO: 108, SEQ ID NO: 109, SEQ ID NO: 113, SEQ ID NO: 119, SEQ ID NO: 120, SEQ ID NO: 126, SEQ ID NO: 127, SEQ ID NO: 131, SEQ ID NO: 132, SEQ ID NO: 141, SEQ ID NO: 142, SEQ ID NO: 147, SEQ ID NO: 148, SEQ ID NO: 153, SEQ ID NO: 154, SEQ ID NO: 157, SEQ ID NO: 158, SEQ ID NO: 163, SEQ ID NO: 164, SEQ ID NO: 172, SEQ ID NO: 173, SEQ ID NO: 180, SEQ ID NO: 181, SEQ ID NO: 190, SEQ ID NO: 191, SEQ ID NO: 197, SEQ ID NO: 198, SEQ ID NO: 205, or SEQ ID NO: 206). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NOs: 7, 8, 17, 18, 24, 25, 34, 35, 42, 43, 52, 53, 60, 61, 66, 67, 76, 77, 83, 84, 92, 93, 102, 103, 108, 109, 113, 119, 120, 126, 127, 131, 132, 141, 142, 147, 148, 153, 154, 157, 158, 163, 164, 172, 173, 180, 181, 190, 191, 197, 198, 205, or 206 including post-translational modifications of that sequence (SEQ ID NO: 7, 8, 17, 18, 24, 25, 34, 35, 42, 43, 52, 53, 60, 61, 66, 67, 76, 77, 83, 84, 92, 93, 102, 103, 108, 109, 113, 119, 120, 126, 127, 131, 132, 141, 142, 147, 148, 153, 154, 157, 158, 163, 164, 172, 173, 180, 181, 190, 191, 197, 198, 205, or 206).

In certain embodiments, the extracellular antigen-binding domain of a presently disclosed CAR cross-competes for binding to DLL3 (e.g., human DLL3) with a reference antibody or an antigen-binding fragment thereof comprising the V_{H} CDR1, CDR2, and CDR3 sequences and the V_{L} CDR1, CDR2, and CDR3 sequences of, for example, any one of the presently disclosed scFvs (e.g.). In certain embodiments, the extracellular antigen-binding domain of a presently disclosed CAR cross-competes for binding to DLL3 (e.g., human DLL3) with a reference antibody or an antigen-binding portion thereof comprising the V_{H} and V_{L} sequences of, for example, any one of the presently disclosed scFvs (e.g., J8, L22, B2, A18, E9, G3, M11, O24, P4, J23, K19, N10, B16-v1, B16-v2, E23, F9, L12, B22, C22, D8, G16, F21, N12, and G23).

In certain embodiments, the extracellular antigen-binding domain of a presently disclosed CAR cross-competes for binding to DLL3 (e.g., human DLL3) with a reference antibody or an antigen-binding portion thereof comprising the V_{H} CDR1, CDR2, and CDR3 sequences and the V_{L} CDR1, CDR2, and CDR3 sequences of scFv J8. For example, the extracellular antigen-binding domain of a presently disclosed CAR cross-competes for binding to DLL3 (e.g., human DLL3) with a reference antibody or an antigen-binding portion thereof comprising a V_{H} CDR1 comprising the amino acid sequence set forth in SEQ ID NO: 1, a V_{H} CDR2 comprising the amino acid sequence set forth in SEQ ID NO: 2; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising amino acids having the sequence set forth in SEQ ID NO: 5; and a V_{L} CDR3 comprising amino acids having the sequence set forth in SEQ ID NO: 6. In certain embodiments, the extracellular antigen-binding domain of a presently disclosed CAR cross-competes for binding to DLL3 (e.g., human DLL3) with a reference antibody or an antigen-binding portion thereof comprising the V_{H} and V_{L} sequences of scFv J8. For example, the extracellular antigen-binding domain of a presently disclosed CAR cross-competes for binding to DLL3 (e.g., human DLL3) with a reference antibody or an antigen-binding portion thereof comprising a V_{H} comprising amino acids having the sequence set forth in SEQ ID NO: 7, and a V_{L} Comprising amino acids having the sequence set forth in SEQ ID NO: 8.

In certain embodiments, the extracellular antigen-binding domain binds to the same epitope region on DLL3 (e.g., human DLL3) as the reference antibody or antigen-binding portion thereof. For example, the extracellular antigen-binding domain of a presently disclosed CAR binds to the same epitope region on DLL3 (e.g., human DLL3) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., J8, L22, B2, A18, E9, G3, M11, O24, P4, J23, K19, N10, B16-v1, B16-v2, E23, F9, L12, B22, C22, D8, G16, F21, N12, and G23). In certain embodiments, the extracellular antigen-binding domain of a presently disclosed CAR binds to the same epitope region on DLL3 (e.g., human DLL3) as a reference antibody or an antigen-binding portion thereof comprising the V_{H} and V_{L} sequences of, for example, any one of the presently disclosed scFvs (e.g., J8, L22, B2, A18, E9, G3, M11, O24, P4, J23, K19, N10, B16-v1, B16-v2, E23, F9, L12, B22, C22, D8, G16, F21, N12, and G23).

In certain embodiments, the extracellular antigen-binding domain cross-competes for binding to DLL3 (e.g., human DLL3) with a reference antibody or an antigen-binding portion thereof. For example, the extracellular antigen-binding domain of a presently disclosed CAR cross-competes for binding to DLL3 (e.g., human DLL3) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., J8, L22, B2, A18, E9, G3, M11, O24, P4, J23, K19, N10, B16-v1, B16-v2, E23, F9, L12, B22, C22, D8, G16, F21, N12, and G23). In certain embodiments, the extracellular antigen-binding domain of a presently disclosed CAR binds to the same epitope region on DLL3 (e.g., human DLL3) as a reference antibody or an antigen-binding portion thereof comprising the V_{H} and V_{L} sequences of, for example, any one of the presently disclosed scFvs (e.g., J8, L22, B2, A18, E9, G3, M11, O24, P4, J23, K19, N10, B16-v1, B16-v2, E23, F9, L12, B22, C22, D8, G16, F21, N12, and G23).

Extracellular antigen-binding domains that cross-compete or compete with the reference antibody or antigen-binding portions thereof for binding to DLL3 (e.g., human DLL3) can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a DLL3 polypeptide, admixing the reference antibody with a test extracellular antigen-binding domain, measuring a second binding of the reference antibody to the DLL3 polypeptide in the presence of the test extracellular antigen-binding domain, and comparing the initial binding with the second binding of the reference antibody, wherein a decreased second binding of the reference antibody to the DLL3 polypeptide in comparison to the initial binding indicates that the test extracellular antigen-binding domain cross-competes with the reference antibody for binding to DLL3, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the DLL3 polypeptide is expressed in cells, e.g., in a flow cytometry test. In certain embodiments, the DLL3 polypeptide is immobilized onto a surface, including a Biacore ship (e.g., in a Biacore test), or other media suitable for surface plasmon resonance analysis. The binding of the reference antibody in the presence of a completely irrelevant antibody (that does not bind to DLL3) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to a DLL3 polypeptide by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to DLL3. In certain embodiments, the assays are performed at room temperature.

In certain embodiments, the antibody-binding assay comprises measuring an initial binding of a test extracellular antigen-binding domain to a DLL3 polypeptide, admixing the test extracellular antigen-binding domain with a reference antibody, measuring a second binding of the test extracellular antigen-binding domain to the DLL3 polypeptide in the presence of the reference antibody, and comparing the initial binding with the second binding of the test extracellular antigen-binding domain, where a decreased second binding of the test extracellular antigen-binding domain to the DLL3 polypeptide in comparison to the initial binding indicates that the test extracellular antigen-binding domain cross-competes with the reference antibody for binding to DLL3, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the DLL3 polypeptide is expressed in cells, e.g., in a flow cytometry test. In certain embodiments, the DLL3 polypeptide is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to DLL3) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to a DLL3 polypeptide is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to DLL3. In certain embodiments, the assays are performed at room temperature.

In certain non-limiting embodiments, the extracellular antigen-binding domain of the presently disclosed CAR comprises a linker connecting the heavy chain variable region and light chain variable region of the extracellular antigen-binding domain. In certain embodiments, the linker comprises or consists of the amino acid sequence set forth in SEQ ID NO: 209. In certain embodiments, the linker comprises or consists of the amino acid sequence set forth in SEQ ID NO: 210. In certain embodiments, the linker comprises or consists of the amino acid sequence set forth in SEQ ID NO: 211. In certain embodiments, the linker comprises or consists of the amino acid sequence set forth in SEQ ID NO: 212. In certain embodiments, the linker comprises or consists of the amino acid sequence set forth in SEQ ID NO: 213. In certain embodiments, the linker comprises or consists of the amino acid sequence set forth in SEQ ID NO: 214.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

### 5.3.2. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-11B) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In certain embodiments, the CAR comprises an extracellular antigen-binding domain that specifically binds to DLL3, a transmembrane domain, and an intracellular signaling domain.

### 5.3.2.1. Extracellular Antigen-Binding Domain of a CAR

The extracellular antigen-binding domain of the CAR can be any extracellular antigen-binding domain disclosed herein, e.g., in Section 4.3.1.

In certain embodiments, the CAR comprises an extracellular antigen-binding domain disclosed in Section 4.3.1.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide.

### 5.3.2.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof). In certain embodiments, the transmembrane domain of the CAR comprises a transmembrane domain of human CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 216) or a fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 216, which is at least 20, or at least 30, or at least 40, or at least 50, and up to 235 amino acids in length. Alternatively or additionally, in non-limiting various embodiments, the CD8 polypeptide comprises or consists of an amino acid sequence of amino acids 1 to 235, 1 to 50, 50 to 100, 100 to 150, 150 to 200, 137 to 209 or 200 to 235 of SEQ ID NO: 216. In certain embodiments, the transmembrane domain of the CAR comprises a CD8 polypeptide comprising or consisting of amino acids 137 to 209 of SEQ ID NO: 216. SEQ ID NO: 216 is provided below.

[SEQ ID NO: 216]
MALPVTALLLPLALLLHAARPSQFRVSPLDRTWNLGETVELKCQVLLSN
PTSGCSWLFQPRGAAASPTELLYLSQNKPKAAEGLDTQRFSGKRLGDTF
VLTLSDERRENEGYYFCSALSNSIMYFSHFVPVELPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the transmembrane domain of the CAR comprises a transmembrane domain of mouse CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 217) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 217, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 217. In certain embodiments, the transmembrane domain of the CAR comprises a CD8 polypeptide comprising or consisting of amino acids 151 to 219 of SEQ ID NO: 217. SEQ ID NO: 217 is provided below.

| [SEQ ID NO: 217] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKES |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVROEGKP |
| 241 | RPSEKIV |

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a transmembrane domain of human CD28 or a fragment thereof. In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID No: 218) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 218 which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 153 to 179, or 200 to 220 of SEQ ID NO: 218. In certain embodiments, the transmembrane domain of the CAR comprises a CD28 polypeptide comprising or consisting of amino acids 153 to 179 of SEQ ID NO: 218. SEQ ID NO: 218 is provided below:

| [SEQ ID NO: 218] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID No: 219) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 219, which is at least 20, or at least 30, or at least 40, or at least 50, and up to 218 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 151 to 177, or 200 to 218 of SEQ ID NO: 219. In certain embodiments, the transmembrane domain of the CAR comprises a CD28 polypeptide comprising or consisting of amino acids 151 to 177 of SEQ ID NO: 219. SEQ ID NO: 219 is provided below:

| [SEQ ID NO: 219] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEfNCDGD FDNETVTERL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLqSDYM NMTPRRPGLT RKPYQPYAPA RDFAAYRP |

In certain non-limiting embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 218 or 219), a portion of a CD8 polypeptide (e.g., a portion of SEQ ID NO: 216 or 217), a variation of any of the foregoing which is at least about 80%, at least about 85%, at least about 90%, at least about 95%, or at least about 100% homologous or identical thereto, or a synthetic spacer sequence.

### 5.3.2.3. Intracellular Signaling Domain of a CAR

In certain embodiments, the CAR comprises an intracellular signaling domain. In certain non-limiting embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 220) or a fragment thereof, and/or may optionally comprise up to one or up to two or up to three conservative amino acid substitutions. In certain non-limiting embodiments, the CD3ζ polypeptide comprises or consists of an amino acid sequence that is a consecutive portion of SEQ ID NO: 220, which is at least 20, or at least 30, or at least 40, or at least 50, and up to 164 amino acids in length. Alternatively or additionally, in non-limiting various embodiments, the CD3ζ polypeptide comprises or consists of an amino acid sequence of amino acids 1 to 164, 1 to 50, 50 to 100, 52 to 164, 100 to 150, or 150 to 164 of SEQ ID NO: 220. In certain embodiments, the intracellular signaling domain of the CAR comprises a CD3ζ polypeptide comprising or consisting of amino acids 52 to 164 of SEQ ID NO: 220. SEQ ID NO: 220 is provided below:

| [SEQ ID NO: 220] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the CAR comprises a CD3 polypeptide comprising or consisting of the amino acid sequence set forth in SEQ ID NO: 221. SEQ ID NO: 221 is provided below.

[SEQ ID NO: 221]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

An exemplary nucleotide sequence encoding the amino acid sequence of SEQ ID NO: 221 is set forth in SEQ ID NO: 222, which is as provided below.

[SEQ ID NO: 222]
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGC

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. In certain embodiments, the intracellular signaling domain of the CAR comprises a co-stimulatory signaling region that comprises a CD28 polypeptide, e.g., an intracellular domain of human CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 218 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 218, which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 218. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 218.

In certain embodiments, the intracellular signaling domain of the CAR comprises a co-stimulatory signaling region that comprises a CD28 polypeptide, e.g., an intracellular domain of mouse CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 219 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 219, which is at least about 20, or at least about 30, or at least about 40, or at least about 50, and up to 218 amino acids in length. Alternatively or additionally, in non-limiting various embodiments, the CD28 polypeptide comprises or consists of an amino acid sequence of amino acids 1 to 218, 1 to 50, 50 to 100, 100 to 150, 150 to 218, 178 to 218, or 200 to 218 of SEQ ID NO: 219. In certain embodiments, the co-stimulatory signaling region of a presently disclosed CAR comprises a CD28 polypeptide that comprises or consists of the amino acids 178 to 218 of SEQ ID NO: 219.

In certain embodiments, the co-stimulatory signaling region of a presently disclosed CAR comprises a CD28 polypeptide comprising a mutated YMNM motif. CD28 is a transmembrane protein that plays a critical role in T cell activation through its function as a costimulatory molecule. CD28 possesses an intracellular domain, which comprises intracellular motifs that are critical for the effective signaling of CD28. In certain embodiments, the CD28 intracellular domain comprises intracellular subdomains (also known as “intracellular motifs”) that regulate signaling pathways post TCR-stimulation. CD28 includes three intracellular motifs: a YMNM motif, and two proline-rick motifs: PRRP motif, and PYAP motif. The CD28 intracellular motifs can serve as docking sites for a number of adaptor molecules that interact with these motifs through their SH2 or SH3 domains. Such interaction transduces downstream signals terminating on transcription factors that regulate gene expression. For example, a native YMNM motif binds to a p85 subunit of a phosphoinositide 3-kinase (PI3K). A native YMNM motif also binds to growth factor receptor-bound protein 2 (Grb2) and/or Grb2-related adaptor protein 2 (GADS). Grb2 binds to Gab1 and Gab2, which in turn can recruit the p85 subunit of a PI3K.

In certain embodiments, a native YMNM motif consists of the amino acid sequence set forth in YMNM (SEQ ID NO: 224). In certain embodiments, a native YMNM motif binds to the p85 subunit of PI3K via a consensus sequence YMxM (SEQ ID NO: 225), wherein x is not an aspartic acid (N). In certain embodiments, a native YMNM motif binds to Grb2 and/or GADs via a consensus sequence YxNx (SEQ ID NO: 226), wherein x is not a methionine (M).

In certain embodiments, the CD28 polypeptide comprising a presently disclosed mutated YMNM motif has reduced recruitment of the p85 subunit of a PI3K as compared to a CD28 molecule comprising a native YMNM motif. In certain embodiments, the p85 subunit of a PI3K does not bind to the mutated YMNM motif, thereby reducing the recruitment of the p85 subunit of a PI3K to the CD28 polypeptide. The mutated YMNM motif that blocks the binding of the p85 subunit of a PI3K retains its binding to Grb2 and/or GADS. Thus, downstream signaling of Grb2/GADS remains intact, e.g., downstream signaling leading to IL-2 secretion remains intact. Such mutated YMNM motif is referred to as “GADS/Grb2-permitting mutant”.

In certain embodiments, the mutated YMNM binds to the p85 subunit of a PI3K, but does not bind to Grb2 and/or GADS. Since the binding of PI3K p85 is retained, the downstream signaling of PI3K retains intact. Since the binding of Grb2/GADS is blocked, the recruitment of PI3K p85 subunit, which is triggered by the binding of Grb2 to Gab1 and Gab2, is reduced or blocked. In addition, the downstream signaling of Grb2/GADS is blocked. Such mutated YMNM motif is referred to as “PI3K-permissive mutant”.

In certain embodiments, the mutated YMNM does not bind to the p85 subunit of a PI3K, and does not bind to Grb2 and/or GADS. Such mutated YMNM motif is referred to as “non-functional mutant”. Non-functional mutants do not provide binding of PI3K, Grb2, or GADS to CD28 at the YMNM motif, but do not preclude these signaling molecules from binding elsewhere in the CD28 molecule.

In certain embodiments, the mutated YMNM retains only one methionine residue of the two methionine residues present in the YMNM motif, i.e. YMxx or YxxM. These motifs potentially modulate signaling via PI3K by limiting how many methionine residues can bind the p85 subunit of PI3K. Such mutated YMNM motif is referred to as “hybrid ‘HEMI’ mutant”.

In certain embodiments, the mutated YMNM motif is a GADS/Grb-2 permitting mutant. In certain embodiments, the mutated YMNM motif consists of the amino acid sequence set forth in YxNx (SEQ ID NO: 226), wherein x is not a methionine (M). In certain embodiments, x is selected from the group consisting of amino acids A, R, N, D, C, E, Q, G, H, I, K, F, P, S, T, W, Y, V, and L. In certain embodiments, the mutated YMNM motif consists of the amino acid sequence set forth in YENV (SEQ ID NO: 227), YSNV (SEQ ID NO: 228), YKNL (SEQ ID NO: 229), YENQ (SEQ ID NO: 230), YKNI (SEQ ID NO: 231), YINQ (SEQ ID NO: 232), YHNK (SEQ ID NO: 233), YVNQ (SEQ ID NO: 234), YLNP (SEQ ID NO: 235), YLNT (SEQ ID NO: 236), YDND (SEQ ID NO: 237), YENI (SEQ ID NO: 238), YENL (SEQ ID NO: 239), YKNQ (SEQ ID NO: 240), YKNV (SEQ ID NO: 241), or YANG (SEQ ID NO: 242). In certain embodiments, the mutated YMNM motif consists of the amino acid sequence set forth in YSNV (SEQ ID NO: 228). In certain embodiments, the mutated YMNM motif consists of the amino acid sequence set forth in YKNI (SEQ ID NO: 231). In certain embodiments, the mutated YMNM motif consists of the amino acid sequence set forth in YENV (SEQ ID NO: 227). In certain embodiments, the mutated YMNM motif consists of the amino acid sequence set forth in YKNL (SEQ ID NO: 229).

In certain embodiments, the mutated YMNM motif is a PI3K-permissive mutant. In certain embodiments, the mutated YMNM motif consists of the amino acid sequence set forth in YMxM (SEQ ID NO: 225), wherein x is not an aspartic acid (N). In certain embodiments, x is selected from the group consisting of amino acids A, R, D, C, E, Q, G, H, I, K, M, F, P, S, T, W, Y, V, and L. In certain embodiments, the mutated YMNM motif consists of the amino acid sequence set forth in YMDM (SEQ ID NO: 243), YMPM (SEQ ID NO: 244), YMRM (SEQ ID NO: 245), or YMSM (SEQ ID NO: 246). In certain embodiments, the mutated YMNM motif consists of the amino acid sequence set forth in YMDM (SEQ ID NO: 243).

In certain embodiments, the mutated YMNM motif consists of the amino acid sequence set forth in YbxM (SEQ ID NO: 247), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YTHM (SEQ ID NO: 248), YVLM (SEQ ID NO: 249), YIAM (SEQ ID NO: 250), YVEM (SEQ ID NO: 251), YVKM (SEQ ID NO: 252), or YVPM (SEQ ID NO: 253).

In certain embodiments, the mutated YMNM motif consists of the amino acid sequence set forth in YMxb (SEQ ID NO: 254), wherein x is not an aspartic acid (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMAP (SEQ ID NO: 255).

Certain mutated YMNM motifs are described in Mol Cell Proteomics. 2010 November; 9(11):2391-404; Virology. 2015 May; 0: 568-577, both of which are incorporated by reference herein in its entirety.

In certain embodiments, the mutated YMNM motif is a hybrid ‘HEMI’ mutant. In certain embodiments, the mutated YMNM motif consists of the amino acid sequence set forth in YMNx (SEQ ID NO: 256) or YxNM (SEQ ID NO: 257), wherein x is not a methionine (M). In certain embodiments, x is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMNV (SEQ ID NO: 258), YENM (SEQ ID NO: 259), YMNQ (SEQ ID NO: 260), YMNL (SEQ ID NO: 261), or YSNM (SEQ ID NO: 262).

In certain embodiments, the mutated YMNM motif is a non-functional mutant. In certain embodiments, the mutated YMNM motif consists of the amino acid sequence Ybxb (SEQ ID NO: 263), wherein x is not an aspartic acid (N), and b is not a methionine (M). In certain embodiments, x is selected from the group consisting of A, R, D, C, E, Q, G, H, I, K, M, F, P, S, T, W, Y, V, and L. In certain embodiments, b is selected from the group consisting of A, R, N, D, C, E, Q, G, H, I, K, F, P, S, T, W, Y, V, and L. In certain embodiments, the mutated YMNM motif consists of the amino acid sequence set forth in YGGG (SEQ ID NO: 264), YAAA (SEQ ID NO: 265), YFFF (SEQ ID NO: 266), YETV (SEQ ID NO: 267), YQQQ (SEQ ID NO: 268), YHAE (SEQ ID NO: 269), YLDL (SEQ ID NO: 270), YLIP (SEQ ID NO: 271), YLRV (SEQ ID NO: 272), YTAV (SEQ ID NO: 273), or YVHV (SEQ ID NO: 274). In certain embodiments, the mutated YMNM motif consists of the amino acid sequence set forth in YGGG (SEQ ID NO: 264).

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YENV (SEQ ID NO: 227), wherein the CD28 polypeptide comprises or consists of the amino acid sequence set forth in SEQ ID NO: 275. SEQ ID NO: 275 is provided below.

[SEQ ID NO: 275]
RSKRSRLLHSDYENVTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YKNI (SEQ ID NO: 231), wherein the CD28 polypeptide comprises or consists of the amino acid sequence set forth in SEQ ID NO: 276. SEQ ID NO: 276 is provided below.

[SEQ ID NO: 276]
RSKRSRLLHSDYKNITPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YMDM (SEQ ID NO: 243), wherein the CD28 polypeptide comprises or consists of the amino acid sequence set forth in SEQ ID NO: 277. SEQ ID NO: 277 is provided below.

[SEQ ID NO: 277]
RSKRSRLLHSDYMDMTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YGGG (SEQ ID NO: 264), wherein the CD28 polypeptide comprises or consists of the amino acid sequence set forth in SEQ ID NO: 278. SEQ ID NO: 278 is provided below.

[SEQ ID NO: 278]
RSKRSRLLHSDYGGGTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YSNV (SEQ ID NO: 228), wherein the CD28 polypeptide comprises or consists of the amino acid sequence set forth in SEQ ID NO: 279. SEQ ID NO: 279 is provided below.

[SEQ ID NO: 279]
RSKRSRLLHSDYSNVTPRRPGPTRKHYQPYAPPRDFAAYRS

In certain embodiments, the intracellular signaling domain of the presently disclosed CAR comprises a first co-stimulatory signaling domain that comprises a CD28 polypeptide comprising a mutated YMNM motif (as disclosed herein), and a second co-stimulatory signaling domain that comprises an intracellular domain of a co-stimulatory molecule. Additional information regarding CARs including CD28 polypeptide comprising a mutated YMNM motif can be found in International Patent Publication No. WO 2021/158850, which is incorporated by reference in its entirety.

In certain embodiments, the intracellular signaling domain of the CAR comprises a co-stimulatory signaling region that comprises a 4-1BB polypeptide, e.g., an intracellular domain of 4-1BB or a fragment thereof. In certain embodiments, the intracellular signaling domain of the CAR comprises a co-stimulatory signaling region that comprises a 4-1BB polypeptide, e.g., an intracellular domain of human 4-1BB or a fragment thereof. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 221) or a fragment thereof, and/or may optionally comprise up to one or up to two or up to three conservative amino acid substitutions. In non-limiting certain embodiments, the 4-1BB polypeptide comprises or consists of an amino acid sequence that is a consecutive portion of SEQ ID NO: 223, which is at least 20, or at least 30, or at least 40, or at least 50, or at least 100, or at least 150, or at least 150, and up to 255 amino acids in length. Alternatively or additionally, in non-limiting various embodiments, the 4-1BB polypeptide comprises or consists of an amino acid sequence of amino acids 1 to 255, 1 to 50, 50 to 100, 100 to 150, 150 to 200, or 200 to 255 of SEQ ID NO: 223. In certain embodiments, the intracellular signaling domain of the CAR comprises a co-stimulatory signaling region that comprises a 4-1BB polypeptide comprising or consisting of an amino acid sequence of amino acids 214 to 255 of SEQ ID NO: 223. SEQ ID NO: 223 is provided below.

| [SEQ ID NO: 223] | |
|---|---|
| 1 | MGNSCYNIVA TLLLVLNFER TRSLQDPCSN CPAGTFCDNN RNQICSPCPP NSFSSAGGQR |
| 61 | TCDICROCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

### 5.3.2.4. Exemplified CARs

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprising amino acids 180 to 220 of SEQ ID NO: 218. In certain embodiments, the V_{H} and V_{L} are linked via a linker comprising or consisting of the amino acid sequence set forth in SEQ ID NO: 210. In certain embodiments, the V_{H} and V_{L} are positioned from the N- to the C-terminus: V_{L}-V_{H}. In certain embodiments, the CAR is designed as “J8-LH_h28z”.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a 4-1BB polypeptide (e.g., an intracellular domain of human 4-1BB or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a 4-1BB polypeptide comprising amino acids 214 to 255 of SEQ ID NO: 223. In certain embodiments, the V_{H} and V_{L} are linked via a linker comprising or consisting of the amino acid sequence set forth in SEQ ID NO: 210. In certain embodiments, the V_{H} and V_{L} are positioned from the N- to the C-terminus: V_{L}-V_{H}. In certain embodiments, the CAR is designed as “J8-LH_hBBz”.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13, and (ii) a V_{L} that comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprising amino acids 180 to 220 of SEQ ID NO: 218. In certain embodiments, the V_{H} and V_{L} are linked via a linker comprising or consisting of the amino acid sequence set forth in SEQ ID NO: 210. In certain embodiments, the V_{H} and V_{L} are positioned from the N- to the C-terminus: V_{H}-V_{L}. In certain embodiments, the CAR is designed as “L22-HL_h28z”.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13, and (ii) a V_{L} that comprises a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a 4-1BB polypeptide (e.g., an intracellular domain of human 4-1BB or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a 4-1BB polypeptide comprising amino acids 214 to 255 of SEQ ID NO: 223. In certain embodiments, the V_{H} and V_{L} are linked via a linker comprising or consisting of the amino acid sequence set forth in SEQ ID NO: 210. In certain embodiments, the V_{H} and V_{L} are positioned from the N- to the C-terminus: V_{H}-V_{L}. In certain embodiments, the CAR is designed as “L22-HL_hBBz”.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprising amino acids 180 to 220 of SEQ ID NO: 218. In certain embodiments, the V_{H} and V_{L} are linked via a linker comprising or consisting of the amino acid sequence set forth in SEQ ID NO: 210. In certain embodiments, the V_{H} and V_{L} are positioned from the N- to the C-terminus: V_{L}-V_{H}. In certain embodiments, the CAR is designed as “B2-LH_h28z”.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a 4-1BB polypeptide (e.g., an intracellular domain of human 4-1BB or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a 4-1BB polypeptide comprising amino acids 214 to 255 of SEQ ID NO: 223. In certain embodiments, the V_{H} and V_{L} are linked via a linker comprising or consisting of the amino acid sequence set forth in SEQ ID NO: 210. In certain embodiments, the V_{H} and V_{L} are positioned from the N- to the C-terminus: V_{L}-V_{H}. In certain embodiments, the CAR is designed as “B2-LH_hBBz”.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide comprising a mutated YMNM motif consisting of the amino acid sequence set forth in YSNV (SEQ ID NO: 228). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprises or consists of the amino acid sequence set forth in SEQ ID NO: 279. In certain embodiments, the V_{H} and V_{L} are linked via a linker comprising or consisting of the amino acid sequence set forth in SEQ ID NO: 210. In certain embodiments, the V_{H} and V_{L} are positioned from the N- to the C-terminus: V_{L}-V_{H}. In certain embodiments, the CAR is designed as “2J8-28YSNVz”.

### 5.3.3. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

### 5.4. Cells

The presently disclosed subject matter provides cells comprising a presently disclosed DLL3-targeted antigen-recognizing receptor (e.g., one disclosed in Section 4.3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cells can be transduced with the presently disclosed DLL3-targeted antigen-recognizing receptor such that the cells express the antigen-recognizing receptor.

In certain embodiments, the presently disclosed cell further comprises a soluble single-chain variable fragment (scFv) that binds a polypeptide that has immunosuppressive activity or immunostimulatory activity. In certain embodiments, immunosuppressive activity refers to induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPα, PD-L1, PD-L2, B7-1, and B7-2. Such polypeptides are present in the tumor microenvironment and inhibit immune responses to neoplastic cells. In various embodiments, inhibiting, blocking, or antagonizing the interaction of immunosuppressive polypeptides and/or their ligands enhances the immune response of the immunoresponsive cell.

In certain embodiments, the immunostimulatory activity refers to induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX-40, 4-IBB, and their corresponding ligands, including B7-1, B7-2, OX-40L, and 4-1BBL. Such polypeptides are present in the tumor microenvironment and activate immune responses to neoplastic cells. In various embodiments, promoting, stimulating, or agonizing pro-inflammatory polypeptides and/or their ligands enhances the immune response of the immunoresponsive cell.

Cells comprising a CAR and a soluble scFv that binds a polypeptide that has immunosuppressive activity or immunostimulatory activity are disclosed in International Patent Publication No. WO 2014/134165, which is incorporated by reference in its entirety.

In certain embodiments, the presently disclosed cell further comprises an exogenous CD40L. Cells comprising a CAR and an exogenous CD40L are disclosed in International Patent Publication No. WO 2014/134165.

Furthermore, in certain embodiments, the presently disclosed cell is engineered to express IL-18. In certain embodiments, the presently disclosed cell further comprises an exogenous IL-18 polypeptide or a fragment thereof. In certain embodiments, the presently disclosed cell further comprises a modified promoter/enhancer at an IL-18 gene locus, which can increase IL-18 gene expression, e.g., a constitutive or inducible promoter is placed to drive IL-18 gene expression. Cells comprising a chimeric receptor and engineered to express IL-18, e.g., comprising an exogenous IL-18 polypeptide or a fragment thereof or a modified promoter/enhancer at an IL-18 gene locus are disclosed in International Patent Publication No. WO2018/027155, which is incorporated by reference in its entirety.

Additionally or alternatively, the presently disclosed cell is engineered to express IL-33. In certain embodiments, the presently disclosed cell further comprises an exogenous IL-33 polypeptide or a fragment thereof. In certain embodiments, the presently disclosed cell further comprises a modified promoter/enhancer at an IL-33 gene locus, which can increase IL-33 gene expression, e.g., a constitutive or inducible promoter placed to drive IL-33 gene expression. Cells comprising a chimeric receptor and engineered to express IL-33, e.g., comprising an exogenous IL-33 polypeptide or a fragment thereof or a modified promoter/enhancer at an IL-33 gene locus are disclosed in International Patent Publication No. WO2019/099479, which is incorporated by reference in its entirety.

Additionally or alternatively, the presently disclosed cell is engineered to express IL-36. In certain embodiments, the presently disclosed cell further comprises an exogenous IL-36 polypeptide or a fragment thereof. In certain embodiments, the presently disclosed cell further comprises a modified promoter/enhancer at an IL-36 gene locus, which can increase IL-36 gene expression, e.g., a constitutive or inducible promoter placed to drive IL-36 gene expression. Cells comprising a chimeric receptor and engineered to express IL-36, e.g., comprising an exogenous IL-36 polypeptide or a fragment thereof or a modified promoter/enhancer at an IL-36 gene locus are disclosed in International Patent Publication No. WO2019/099483, which is incorporated by reference in its entirety.

### 5.4.1. Exemplified Cells

In certain embodiments, the cell comprises an antigen-recognizing receptor. In certain embodiments, the antigen-recognizing receptor is a CAR. In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprising amino acids 180 to 220 of SEQ ID NO: 218.

In certain embodiments, the cell comprises an antigen-recognizing receptor. In certain embodiments, the antigen-recognizing receptor is a CAR. In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide comprising a mutated YMNM motif consisting of the amino acid sequence set forth in YSNV (SEQ ID NO: 228). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprises or consists of the amino acid sequence set forth in SEQ ID NO: 279.

In certain embodiments, the cell comprises an antigen-recognizing receptor and an exogenous IL-18 polypeptide. In certain embodiments, the antigen-recognizing receptor is a CAR. In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprising amino acids 180 to 220 of SEQ ID NO: 218. In certain embodiments, the exogenous IL-18 polypeptide is a human IL-18 polypeptide.

In certain embodiments, the cell comprises an antigen-recognizing receptor and an exogenous IL-18 polypeptide. In certain embodiments, the antigen-recognizing receptor is a CAR. In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide comprising a mutated YMNM motif consisting of the amino acid sequence set forth in YSNV (SEQ ID NO: 228). In certain embodiments, the transmembrane domain comprises a CD28 polypeptide that comprises amino acids 153 to 179 of SEQ ID NO: 218. In certain embodiments, the intracellular signaling domain comprises (i) a CD3ζ polypeptide comprising the amino acid sequence set forth in SEQ ID NO: 221, and (ii) a co-stimulatory signaling region comprising a CD28 polypeptide comprises or consists of the amino acid sequence set forth in SEQ ID NO: 279. In certain embodiments, the exogenous IL-18 polypeptide is a human IL-18 polypeptide.

### 5.5. Nucleic Acid Compositions and Vectors

The present discloses subject matter provides a nucleic acid encoding a presently disclosed DLL3-targeted antigen-recognizing receptor (e.g., one disclosed in Section 4.3). Further provided are nucleic acid compositions comprising the nucleic acids disclosed herein. Also provided are cells comprising such nucleic acid compositions.

In certain embodiments, the nucleic acid composition further comprises a promoter that is operably linked to the presently disclosed DLL3-targeted antigen-recognizing receptor.

In certain embodiments, the promoter is endogenous or exogenous. In certain embodiments, the exogenous promoter is selected from an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, the promoter is an inducible promoter. In certain embodiment, the inducible promoter is selected from a NFAT transcriptional response element (TRE) promoter, a CD69 promoter, a CD25 promoter, and an IL-2 promoter.

The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed DLL3-targeted antigen-recognizing receptor (e.g., a CAR), a retroviral vector can be employed for transduction, however any other suitable viral vector or non-viral delivery system can be used. The antigen-recognizing receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995) 107:77S-83S). Retroviral vectors are particularly well developed andhave been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

### 5.5.1. Methods of Delivering

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding an hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1(2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, 0-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 5.6. Polypeptides

The presently disclosed subject matter provides methods for optimizing an amino acid sequence or a nucleic acid sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, DLL3, CD8, CD28, 4-1BB, and CD3ζ). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., 3 or 7 amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 5.7. Formulations and Administration

The presently disclosed subject matter provides compositions comprising the presently disclosed cells. In certain embodiments, the compositions are pharmaceutical compositions further comprising a pharmaceutically acceptable carrier. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for treating or ameliorating a disease or disorder. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein and, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, subcutaneous administration, intranodal administration, intratumoral administration, intrathecal administration, intravitreal administration, intrapleural administration, intraosseous administration, intraperitoneal administration, pleural administration, and direct administration to the subject.

Additionally or alternatively, the presently disclosed subject matter also provides compositions comprising lipid nanoparticles (e.g., described in Section 5.5.1) including a nucleic acid or a nucleic acid composition disclosed herein. Compositions comprising the presently disclosed lipid nanoparticles can be conveniently provided as sterile and/or pyrogen-free. Compositions can be prepared to meet the standards of the United States Pharmacopoeia (USP), the European Pharmacopoeia (EP), the British Pharmacopoeia, and/or the International Pharmacopoeia.

Compositions including the presently disclosed lipid nanoparticles can include pharmaceutically acceptable excipients. Non-limiting examples of pharmaceutically acceptable excipients include inert diluents, dispersing agents, granulating agents, surface active agents, emulsifiers, disintegrating agents, binding agents, preservatives, buffering agents, lubricating agents, and/or oils. Furthermore, excipients such as cocoa butter and suppository waxes, coloring agents, coating agents, sweetening, flavoring, and/or perfuming agents can be present in the composition.

In certain embodiments, compositions including the presently disclosed lipid nanoparticles can be prepared as injectable preparations. These injectable preparations can include pharmaceutically acceptable vehicles and solvents including, without any limitation, water, Ringer's solution, U.S.P., isotonic sodium chloride solution, and/or oils (e.g., oleic acid). In certain embodiments, injectable preparations comprising the presently disclosed lipid nanoparticles can include a liquid suspension of crystalline or amorphous material with poor water solubility. Use of these poor water solubility materials allows to slow absorption from subcutaneous or intramuscular injection. Alternatively or additionally, compositions including the presently disclosed lipid nanoparticles can be prepared for rectal or vaginal administration, oral administration, topical and/or transdermal administration, intradermal administration, pulmonary administration, nasal administration, buccal administration, or ophthalmic administration. Additional information on various ways for formulating and preparing pharmaceutical compositions including the presently disclosed lipid nanoparticles can be found in Remington: The Science and Practice of Pharmacy, 22nd Edition, A. R. Gennaro, Lippincott, Williams & Wilkins, Baltimore, Md., 2012.

In certain embodiments, the compositions including the presently disclosed lipid nanoparticles can be formulated for controlled release or sustained release. As used herein, the term “controlled release” refers to a pharmaceutical composition or compound release profile that conforms to a particular pattern of release to effect a therapeutic outcome. As used herein, the term “sustained release” refers to a pharmaceutical composition or compound that conforms to a release rate over a specific period of time. The period of time may include, but is not limited to, hours, days, weeks, months and years.

Compositions comprising the presently disclosed lipid nanoparticles can be provided systemically or directly to a subject for inducing and/or enhancing an immune response to an antigen and/or treating and/or preventing a tumor, e.g., a tumor associated with MUC16. In certain embodiments, the presently disclosed lipid nanoparticles or compositions comprising thereof are provided in vivo to immunoresponsive cells. In certain embodiments, the presently disclosed lipid nanoparticles or compositions comprising thereof are directly injected into an organ of interest (e.g., an organ affected by a neoplasia). Alternatively, the presently disclosed lipid nanoparticles or compositions comprising thereof are provided indirectly to the organ of interest, for example, by administration into the circulatory system (e.g., the tumor vasculature). In certain embodiments, the presently disclosed lipid nanoparticles or compositions comprising thereof are provided ex vivo to immunoresponsive cells. Expansion and differentiation agents can be provided prior to, during or after administration of the lipid nanoparticles or compositions to increase production of cells (e.g., T cells or NK cells) ex vivo or in vivo.

The presently disclosed lipid nanoparticles can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of the presently disclosed lipid nanoparticles are administered to a subject. In certain embodiments, between about 0.005 mg/kg to about 2.5 mg/kg, from about 0.1 mg/kg to about 1 mg/kg, or from about 0.05 mg/kg to about 1 mg/kg of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art. Dosages can be readily adjusted by those skilled in the art (e.g., a decrease in purity may require an increase in dosage).

### 5.8. Methods of Treatment

The presently disclosed cells and compositions comprising thereof can be used for treating or ameliorating a disease or disorder in a subject. In certain embodiments, the disease or disorder is associated with DLL3. In certain embodiments, the disease or disorder is associated with overexpression of DLL3.

In certain embodiments, the method comprises administering to a subject in need thereof the presently disclosed cells or compositions comprising thereof. In certain embodiments, the cell is a T cell. The T cell can be a CD4⁺ T cell or a CD8⁺ T cell. In certain embodiments, the T cell is a CD4⁺ T cell.

Additionally or alternatively, the presently disclosed lipid nanoparticles and compositions comprising thereof can be used for treating or ameliorating a disease or disorder in a subject. In certain embodiments, the disease or disorder is associated with DLL3. In certain embodiments, the disease or disorder is associated with overexpression of DLL3. In certain embodiments, the method comprises administering to a subject in need thereof the presently disclosed lipid nanoparticles or compositions comprising thereof.

For treatment, the amount administered is an amount effective in producing the desired effect. An effective amount can be provided in one or a series of administrations. An effective amount can be provided in a bolus or by continuous perfusion.

In certain embodiments, the disease or disorder is a tumor. In certain embodiments, the presently disclosed cells and compositions can reduce tumor burden, reduce the number of tumor cells, reduce tumor size, and/or eradicate the tumor in the subject, and/or increase or lengthen survival of the subject. In certain embodiments, the tumor is cancer.

Non-limiting examples of diseases and disorders include neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. Non-limiting examples of neuroendocrine tumors of the lung include pulmonary neuroendocrine cancer (including typical carcinoid tumors, and atypical carcinoid tumors), large cell neuroendocrine carcinoma, and small-cell lung cancer. In certain embodiments, the tumor is small-cell lung cancer. In certain embodiments, the melanoma is uveal melanoma. In certain embodiments, the breast cancer is triple negative breast cancer.

Further modification can be introduced to the DLL3-specific CAR-expressing engineered immune cells (e.g., T cells) to avert or minimize the risks of immunological complications (known as “malignant T-cell transformation”), e.g., graft versus-host disease (GvHD). Modification of the engineered immune cells can include engineering a suicide gene into the DLL3-specific CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the C-terminus of the intracellular domain of the DLL3-specific CAR. The suicide gene can be included within the vector comprising nucleic acids encoding the presently disclosed DLL3-specific CARs. The incorporation of a suicide gene into a presently disclosed DLL3-specific CAR gives an added level of safety with the ability to eliminate the majority of CAR T cells within a very short time period. A presently disclosed engineered immune cell (e.g., a T cell) incorporated with a suicide gene can be pre-emptively eliminated at a given time point post CAR T cell infusion, or eradicated at the earliest signs of toxicity.

### 5.9. Kits

The presently disclosed subject matter provides kits for or ameliorating a disease or disorder in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes a nucleic acid molecule encoding a presently disclosed DLL3-targeted antigen-recognizing receptor (e.g., a CAR).

If desired, the cells and/or nucleic acid molecules are provided together with instructions for administering the cells or nucleic acid molecules to a subject having or at risk of developing a disease or disorder. The instructions generally include information about the use of the composition for the treatment and/or prevention of a tumor or neoplasm. In certain embodiments, the instructions include at least one of the following: description of the therapeutic agent; dosage schedule and administration for treatment or prevention of a tumor or neoplasm; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides an antigen-recognizing receptor, comprising an extracellular antigen-binding domain, a transmembrane domain, and an intracellular signaling domain, wherein the extracellular antigen-binding domain specifically binds to DLL3, wherein the extracellular antigen-binding domain comprises:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof;
(b) a heavy chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof;
(c) a heavy chain variable region comprising a CDR1 comprising the amino acid sequence set forth in SEQ ID NO: 21 or a conservative modification thereof, CDR2 comprising the amino acid sequence set forth in SEQ ID NO: 2 or a conservative modification thereof, and a CDR3 comprising the amino acid sequence set forth in SEQ ID NO: 22 or a conservative modification thereof;
(d) a heavy chain variable region comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof;
(e) a heavy chain variable region comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof;
(f) a heavy chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof;
(g) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof;
(h) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and CDR3 comprising the amino acid sequence set forth in SEQ ID NO: 64 or a conservative modification thereof;
(i) a heavy chain variable region comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof;
(j) a heavy chain variable region comprising a CDR1 comprising the amino acid sequence set forth in SEQ ID NO: 21 or a conservative modification thereof, a comprising the amino acid sequence set forth in SEQ ID NO: 80 or a conservative modification thereof, and a CDR3 comprising the amino acid sequence set forth in SEQ ID NO: 81 or a conservative modification thereof;
(k) a heavy chain variable region comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof;
(l) a heavy chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof;
(m) a heavy chain variable region comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof;
(n) a heavy chain variable region comprising a CDR1 comprising the amino acid sequence set forth in SEQ ID NO: 95 or a conservative modification thereof, CDR2 comprising the amino acid sequence set forth in SEQ ID NO: 115 or a conservative modification thereof, and a CDR3 comprising the amino acid sequence set forth in SEQ ID NO: 116 or a conservative modification thereof;
(o) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof;
(p) a heavy chain variable region comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof;
(q) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof;
(r) a heavy chain variable region comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof;
(s) a heavy chain variable region comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof;
(t) a heavy chain variable region comprising a CDR1 comprising the amino acid sequence set forth in SEQ ID NO: 96 or a conservative modification thereof, CDR2 comprising the amino acid sequence set forth in SEQ ID NO: 167 or a conservative modification thereof, and a CDR3 comprising the amino acid sequence set forth in SEQ ID NO: 168 or a conservative modification thereof;
(u) a heavy chain variable region comprising a CDR1 comprising the amino acid sequence set forth in SEQ ID NO: 21 or a conservative modification thereof, a CDR2 comprising the amino acid sequence set forth in SEQ ID NO: 176 or a conservative modification thereof, and a CDR3 comprising the amino acid sequence set forth in SEQ ID NO: 177 or a conservative modification thereof;
(v) a heavy chain variable region comprising a CDR1 comprising the amino acid sequence set forth in SEQ ID NO: 184 or a conservative modification thereof, a CDR2 comprising the amino acid sequence set forth in SEQ ID NO: 185 or a conservative modification thereof, and a CDR3 comprising the amino acid sequence set forth in SEQ ID NO: 186 or a conservative modification thereof;
(w) a heavy chain variable region comprising a CDR1 comprising the amino acid sequence set forth in SEQ ID NO: 11 or a conservative modification thereof, a CDR2 comprising the amino acid sequence set forth in SEQ ID NO: 194 or a conservative modification thereof, and a CDR3 comprising the amino acid sequence set forth in SEQ ID NO: 195 or a conservative modification thereof; or
(x) a heavy chain variable region comprising a CDR1 comprising the amino acid sequence set forth in SEQ ID NO: 201 or a conservative modification thereof, a CDR2 comprising the amino acid sequence set forth in SEQ ID NO: 202 or a conservative modification thereof, and a CDR3 comprising the amino acid sequence set forth in SEQ ID NO: 203 or a conservative modification thereof.

A2. The foregoing antigen-recognizing receptor of A1, wherein the extracellular antigen-binding domain is a single-chain variable fragment (scFv).

A3. The foregoing antigen-recognizing receptor of A2, wherein the extracellular antigen-binding domain is a human scFv.

A4. The foregoing antigen-recognizing receptor of A1, wherein the extracellular antigen-binding domain is a Fab, which is optionally crosslinked.

A5. The foregoing antigen-recognizing receptor of A1, wherein the extracellular antigen-binding domain is a F(ab)₂.

A6. The foregoing antigen-recognizing receptor of any one of A2-A5, wherein one or more of the scFv, Fab and F(ab)₂ are comprised in a fusion protein with a heterologous sequence to form the extracellular antigen-binding domain.

A7. The foregoing antigen-recognizing receptor of any one of A1-A6, wherein the extracellular antigen-binding domain comprises:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof;
(b) a heavy chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; or
(c) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof.

A8. The foregoing antigen-recognizing receptor of any one of A1-A7, wherein the extracellular antigen-binding domain comprises:
(a) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof;
(b) a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising SEQ ID NO: 16 or a conservative modification thereof;
(c) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof;
(d) a light chain variable region comprising a CDR1 comprising the amino acid sequence set forth in SEQ ID NO: 31 or a conservative modification thereof, a CDR2 comprising SEQ ID NO: 32 or a conservative modification thereof, and a CDR3 comprising the amino acid sequence set forth in SEQ ID NO: 33 or a conservative modification thereof;
(e) a light chain variable region comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof;
(f) a light chain variable region comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof;
(g) a light chain variable region comprising a CDR1 comprising the amino acid sequence set forth in SEQ ID NO: 57 or a conservative modification thereof, a 1 CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(h) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof;
(i) a light chain variable region comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof;
(j) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof;
(k) a light chain variable region comprising a CDR1 comprising the amino acid sequence set forth in SEQ ID NO: 90 or a conservative modification thereof, a CDR2 comprising the amino acid sequence set forth in SEQ ID NO: 280 or a conservative modification thereof, and a CDR3 comprising the amino acid sequence set forth in SEQ ID NO: 91 or a conservative modification thereof;
(l) a light chain variable region comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof;
(m) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification thereof;
(n) a light chain variable region comprising a CDR1 comprising the amino acid sequence set forth in SEQ ID NO: 117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification thereof;
(o) a light chain variable region comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification thereof;
(p) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification thereof;
(q) a light chain variable region comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification thereof;
(r) a light chain variable region comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification thereof;
(s) a light chain variable region comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(t) a light chain variable region comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification thereof;
(u) a light chain variable region comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification thereof;
(v) a light chain variable region comprising a CDR1 comprising the amino acid sequence set forth in SEQ ID NO: 178 or a conservative modification thereof, a CDR2 comprising the amino acid sequence set forth in SEQ ID NO: 50 or a conservative modification thereof, and a CDR3 comprising the amino acid sequence set forth in SEQ ID NO: 179 or a conservative modification thereof;
(w) a light chain variable region comprising a CDR1 comprising the amino acid sequence set forth in SEQ ID NO: 187 or a conservative modification thereof, a CDR2 comprising the amino acid sequence set forth in SEQ ID NO: 188 or a conservative modification thereof, and a CDR3 comprising the amino acid sequence set forth in SEQ ID NO: 189 or a conservative modification thereof;
(x) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 196 or a conservative modification thereof; or
(y) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 204 or a conservative modification thereof.

A9. The foregoing antigen-recognizing receptor of any one of A1-A8, wherein the
(a) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof;
(b) a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising SEQ ID NO: 16 or a conservative modification thereof;
(c) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof.

A10. The foregoing antigen-recognizing receptor of any one of A1-A9, wherein the extracellular antigen-binding domain comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
(c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
(d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39, and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
(f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
(h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
(i) a heavy chain variable region comprising a CDR1 comprising the amino acid sequence set forth in SEQ ID NO: 70, a CDR2 comprising the amino acid sequence set forth in SEQ ID NO: 71 and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
(j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and a region CDR3 comprising the amino acid sequence set forth in SEQ ID NO: 82;
(k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 280, and a CDR3 comprising the amino acid sequence set forth in SEQ ID NO: 91;
(l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
(m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
(o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116 and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
(p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
(p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a region CDR3 comprising the amino acid sequence set forth in SEQ ID NO: 125;
(q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
(r) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
(s) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
(t) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(u) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
(v) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
(w) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171;
(x) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177; and a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 79;
(y) a heavy chain variable region comprising a CDR1 comprising the amino acid sequence set forth in SEQ ID NO: 184, a heavy chain variable region CDR2 comprising the amino acid sequence set forth in SEQ ID NO: 185, and a heavy chain variable region CDR3 comprising the amino acid sequence set forth in SEQ ID NO: 186; and a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189;
(z) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a light chain variable region comprising a CDR1 comprising the amino acid sequence set forth in SEQ ID NO: 4, a li CDR2 comprising the amino acid sequence set forth in SEQ ID NO: 5, and a CDR3 comprising the amino acid sequence set forth in SEQ ID NO: 196; or
(aa) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

A11. The foregoing antigen-recognizing receptor of any one of A1-A10, wherein the extracellular antigen-binding domain comprises:
(a) a heavy chain variable region comprising a CDR1 comprising the amino acid sequence set forth in SEQ ID NO: 1, a CDR2 comprising the amino acid sequence set forth in SEQ ID NO: 2 and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; or
(c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23.

A12. The foregoing antigen-recognizing receptor of any one of A1-A11, wherein the extracellular antigen-binding domain comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

A13. The foregoing antigen-recognizing receptor of any one of A1-A12, wherein the extracellular antigen-binding domain comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

A14. The foregoing antigen-recognizing receptor of any one of A1-A13, wherein the extracellular antigen-binding domain comprises a heavy chain variable region comprising the amino acid sequence set forth in SEQ ID NO: 7, SEQ ID NO: 17, or SEQ ID NO: 24.

A15. The foregoing antigen-recognizing receptor of any one of A1-A14, wherein the extracellular antigen-binding domain comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A16. The foregoing antigen-recognizing receptor of any one of A1-A15, wherein the extracellular antigen-binding domain comprises a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A17. The foregoing antigen-recognizing receptor of any one of A1-A16, wherein the extracellular antigen-binding domain comprises a light chain variable region comprising the amino acid sequence set forth in SEQ ID NO: 8, SEQ ID NO: 18, or SEQ ID NO: 25.

A18. The foregoing antigen-recognizing receptor of any one of A1-A17, wherein the extracellular antigen-binding domain comprises: (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A19. The foregoing antigen-recognizing receptor of any one of A1-A18, wherein the extracellular antigen-binding domain comprises: (a) a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and (b) a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

A20. The foregoing antigen-recognizing receptor of any one of A1-A19, wherein the extracellular antigen-binding domain comprises: (a) a heavy chain variable region comprising the amino acid sequence set forth in SEQ ID NO: 7, SEQ ID NO: 17, or SEQ ID NO: 24; and (b) a light chain variable region comprising the amino acid sequence set forth in SEQ ID NO: 8, SEQ ID NO: 18, or SEQ ID NO: 25.

A21. The foregoing antigen-recognizing receptor of any one of A1-A20, wherein the extracellular antigen-binding domain comprises:
(a) a heavy chain variable region comprising the amino acid sequence set forth in SEQ ID NO: 7, and a light chain variable region comprising the amino acid sequence set forth in SEQ ID NO: 8;
(b) a heavy chain variable region comprising the amino acid sequence set forth in SEQ ID NO: 17, and a light chain variable region comprising the amino acid sequence set forth in SEQ ID NO: 18;
(c) a heavy chain variable region comprising the amino acid sequence set forth in SEQ ID NO: 24, and a light chain variable region comprising the amino acid sequence set forth in SEQ ID NO: 25;
(d) a heavy chain variable region comprising the amino acid sequence set forth in SEQ ID NO: 34, and a light chain variable region comprising the amino acid sequence set forth in SEQ ID NO: 35;
(e) a heavy chain variable region comprising the amino acid sequence set forth in SEQ ID NO: 42, and a light chain variable region comprising the amino acid sequence set forth in SEQ ID NO: 43;
(f) a heavy chain variable region comprising the amino acid sequence set forth in SEQ ID NO: 52, and a light chain variable region comprising the amino acid sequence set forth in SEQ ID NO: 53;
(g) a heavy chain variable region comprising the amino acid sequence set forth in SEQ ID NO: 60, and a light chain variable region comprising the amino acid sequence set forth in SEQ ID NO: 61;
(h) a heavy chain variable region comprising the amino acid sequence set forth in SEQ ID NO: 66, and a light chain variable region comprising the amino acid sequence set forth in SEQ ID NO: 67;
(i) a heavy chain variable region comprising the amino acid sequence set forth in SEQ ID NO: 76, and a light chain variable region comprising the amino acid sequence set forth in SEQ ID NO: 77;
(j) a heavy chain variable region comprising the amino acid sequence set forth in SEQ ID NO: 83, and a light chain variable region comprising the amino acid sequence set forth in SEQ ID NO: 84;
(k) a heavy chain variable region comprising the amino acid sequence set forth in SEQ ID NO: 92, and a light chain variable region comprising the amino acid sequence set forth in SEQ ID NO: 93; and
(l) a heavy chain variable region comprising the amino acid sequence set forth in SEQ ID NO: 102, and a light chain variable region comprising the amino acid sequence set forth in SEQ ID NO: 103.
(m) a heavy chain variable region comprising the amino acid sequence set forth in SEQ ID NO: 108, and a light chain variable region comprising the amino acid sequence set forth in SEQ ID NO: 109;
(n) a heavy chain variable region comprising the amino acid sequence set forth in SEQ ID NO: 108, and a light chain variable region comprising the amino acid sequence set forth in SEQ ID NO: 113;
(o) a heavy chain variable region comprising the amino acid sequence set forth in SEQ ID NO: 119, and a light chain variable region comprising the amino acid sequence set forth in SEQ ID NO: 120;
(p) a heavy chain variable region comprising the amino acid sequence set forth in SEQ ID NO: 126, and a light chain variable region comprising the amino acid sequence set forth in SEQ ID NO: 127;
(q) a heavy chain variable region comprising the amino acid sequence set forth in SEQ ID NO: 131, and a light chain variable region comprising the amino acid sequence set forth in SEQ ID NO: 132;
(r) a heavy chain variable region comprising the amino acid sequence set forth in SEQ ID NO: 141, and a light chain variable region comprising the amino acid sequence set forth in SEQ ID NO: 142;
(s) a heavy chain variable region comprising the amino acid sequence set forth in SEQ ID NO: 147, and a light chain variable region comprising the amino acid sequence set forth in SEQ ID NO: 148;
(t) a heavy chain variable region comprising the amino acid sequence set forth in SEQ ID NO: 153, and a light chain variable region comprising the amino acid sequence set forth in SEQ ID NO: 154;
(u) a heavy chain variable region comprising the amino acid sequence set forth in SEQ ID NO: 157, and a light chain variable region comprising the amino acid sequence set forth in SEQ ID NO: 158;
(v) a heavy chain variable region comprising the amino acid sequence set forth in SEQ ID NO: 163, and a light chain variable region comprising the amino acid sequence set forth in SEQ ID NO: 164;
(w) a heavy chain variable region comprising the amino acid sequence set forth in SEQ ID NO: 172, and a light chain variable region comprising the amino acid sequence set forth in SEQ ID NO: 173;
(x) a heavy chain variable region comprising the amino acid sequence set forth in SEQ ID NO: 180, and a light chain variable region comprising the amino acid sequence set forth in SEQ ID NO: 181;
(y) a heavy chain variable region comprising the amino acid sequence set forth in SEQ ID NO: 190, and a light chain variable region comprising the amino acid sequence set forth in SEQ ID NO: 191;
(z) a heavy chain variable region comprising the amino acid sequence set forth in SEQ ID NO: 197, and a light chain variable region comprising the amino acid sequence set forth in SEQ ID NO: 198; or
(aa) a heavy chain variable region comprising the amino acid sequence set forth in SEQ ID NO: 205, and a light chain variable region comprising the amino acid sequence set forth in SEQ ID NO: 206.

A22. The foregoing antigen-recognizing receptor of any one of A1-A21, wherein the extracellular antigen-binding domain comprises:
(a) a heavy chain variable region comprising the amino acid sequence set forth in SEQ ID NO: 7, and a light chain variable region comprising the amino acid sequence set forth in SEQ ID NO: 8;
(b) a heavy chain variable region comprising the amino acid sequence set forth in SEQ ID NO: 17, and a light chain variable region comprising the amino acid sequence set forth in SEQ ID NO: 18; or
(c) a heavy chain variable region comprising the amino acid sequence set forth in SEQ ID NO: 24, and a light chain variable region comprising the amino acid sequence set forth in SEQ ID NO: 25.

A23. The foregoing antigen-recognizing receptor of any one of A1-A22, wherein the extracellular antigen-binding domain comprises a linker between a heavy chain variable region and a light chain variable region of the extracellular antigen-binding domain.

A24. The foregoing antigen-recognizing receptor of A23, wherein the linker comprises or consists of the amino acid sequence set forth in SEQ ID NO: 209, SEQ ID NO: 210, SEQ ID NO: 211, SEQ ID NO: 212, SEQ ID NO: 213, or SEQ ID NO: 214.

A25. The foregoing antigen-recognizing receptor of any one of A1-A24, wherein the extracellular antigen-binding domain comprises a signal peptide that is covalently joined to the 5′ terminus of the extracellular antigen-binding domain.

A26. The foregoing antigen-recognizing receptor of any one of A1-A25, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

A27. The foregoing antigen-recognizing receptor of any one of A1-A26, wherein the intracellular signaling domain comprises a CD3ζ polypeptide.

A28. The foregoing antigen-recognizing receptor of A27, wherein the CD3ζ polypeptide comprises or consists of the amino acid sequence set forth in SEQ ID NO: 221.

A29. The foregoing antigen-recognizing receptor of any one of A1-A27, wherein the intracellular signaling domain further comprises at least one co-stimulatory signaling region.

A30. The foregoing antigen-recognizing receptor of A28, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

A31. The foregoing antigen-recognizing receptor of A30, wherein the at least one co-stimulatory signaling region comprises a CD28 polypeptide.

A32. The foregoing antigen-recognizing receptor of A31, wherein the CD28 polypeptide comprises or consists of amino acids 180 to 220 of SEQ ID NO: 7.

A33. The foregoing antigen-recognizing receptor of A31, wherein the CD28 polypeptide comprises a mutated YMNM motif.

A34. The foregoing antigen-recognizing receptor of A31 or A33, wherein the CD28 polypeptide comprises or consists of the amino acid sequence set forth in SEQ ID NO: 275, SEQ ID NO: 276, SEQ ID NO: 277, SEQ ID NO: 278, or SEQ ID NO: 279.

A35. The foregoing antigen-recognizing receptor of any one of A1-A34, wherein the antigen-recognizing receptor is a chimeric antigen receptor (CAR), or a T-cell like fusion protein.

A36. The foregoing antigen-recognizing receptor of any one of A1-A35, wherein the antigen-recognizing receptor is a CAR.

A37. The foregoing antigen-recognizing receptor of any one of A1-A36, wherein the antigen-recognizing receptor is recombinantly expressed.

A38. The foregoing antigen-recognizing receptor of any one of A1-A37, wherein the antigen-recognizing receptor is expressed from a vector.

A39. The foregoing antigen-recognizing receptor of A38, wherein the vector is a γ-retroviral vector.

B1. In certain non-limiting embodiments, the presently disclosed subject matter provides a cell comprising the antigen-recognizing receptor of any one of claims 1-39.

B2. The foregoing cell of B1, wherein the cell is transduced with the antigen-recognizing receptor.

B3. The foregoing cell of B1 or B2, wherein the antigen-recognizing receptor is constitutively expressed on the surface of the cell.

B4. The foregoing cell of any one of B1-B3, further comprising an exogenous IL-18 polypeptide.

B5. The foregoing cell of B4, wherein the exogenous IL-18 polypeptide is a human IL-18 polypeptide.

B6. The foregoing cell of any one of B1-B5, wherein the cell is an immunoresponsive cell.

B7. The foregoing cell of any one of B1-B6, wherein the cell is a cell of the lymphoid lineage or a cell of the myeloid lineage.

B8. The foregoing cell of any one of B1-B7, wherein the cell is selected from the group consisting of a T cell, a Natural Killer (NK) cell, and a stem cell from which a lymphoid cell may be differentiated.

B9. The foregoing cell of any one of B1-B8, wherein the cell is a T cell.

B10. The foregoing cell of B8 or B9, wherein the T cell is a cytotoxic T lymphocyte (CTL) or a regulatory T cell.

B11. The foregoing cell of B8, wherein the stem cell is a pluripotent stem cell.

B12. The foregoing cell of B11, wherein the pluripotent stem cell is an embryoid stem cell or an induced pluripotent stem cell.

C. In certain embodiments, the presently disclosed subject matter provides a nucleic acid encoding the antigen-recognizing receptor of any one of A1-A39.

D1. In certain embodiments, the presently disclosed subject matter provides a vector comprising the nucleic acid of any one of C.

D2. The foregoing vector of claim D1, wherein the vector is a γ-retroviral vector.

E1. In certain embodiments, the presently disclosed subject matter provides a host cell expressing the nucleic acid of C.

E2. The foregoing host cell of claim 55, wherein the host cell is a T cell.

F1. In certain embodiments, the presently disclosed subject matter provides a composition comprising the cell of any one of B1-B12.

F2. The foregoing composition of F1, which is a pharmaceutical composition further comprising a pharmaceutically acceptable carrier.

G. In certain embodiments, the presently disclosed subject matter provides a lipid nanoparticle comprising the nucleic acid of C.

H1. In certain embodiments, the presently disclosed subject matter provides a composition comprising the lipid nanoparticle of G.

H2. The foregoing composition of H1, which is a pharmaceutical composition further comprising a pharmaceutically acceptable carrier.

I1. In certain embodiments, the presently disclosed subject matter provides a method of treating or ameliorating a disease or disorder in a subject, comprising administering to the subject the presently disclosed cell of any one of B1-B12, or the composition of any one of claims F1, F2, H1, or H2.

I2. The foregoing method of I1, wherein the disease or disorder is a tumor.

I3. The foregoing method of I2, wherein the tumor is cancer.

I4. The foregoing method of any one of I1-I3, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

I5. The foregoing method of I4, wherein the neuroendocrine tumors of the lung are selected from the group consisting of pulmonary neuroendocrine cancer, large cell neuroendocrine carcinoma, and small-cell lung cancer.

I6. The foregoing method of I5, wherein the tumor is small-cell lung cancer.

I7. The foregoing method of any one of I1-I6, wherein the subject is a human.

J1. In certain embodiments, the presently disclosed subject matter provides a kit for treating or ameliorating a disease or disorder in a subject, comprising the cell of any one of B1-B12, the nucleic acid of C, the lipid nanoparticle of F, or the composition of any one of F1, F2, H1, or H2.

J2. The foregoing kit of J1, wherein the kit further comprises written instructions for using the cell or composition for treating or ameliorating a disease or disorder in a subject.

K. In certain embodiments, the presently disclosed subject matter provides a method for producing a DLL3-targeted antigen-recognizing receptor of any one of A1-A39, comprising introducing into the cell a nucleic acid that encodes the antigen-recognizing receptor.

L1. In certain embodiments, the presently disclosed subject matter provides the cell of any one of B1-B12 or the composition of any one of F1, F2, H1, or H2 for use in treating or ameliorating a disease or disorder in a subject.

L2. The foregoing cell or the foregoing composition for use in L1, wherein the disease or disorder is a tumor.

L3. The foregoing cell or the foregoing composition for use in L2, wherein the tumor is cancer.

L4. The foregoing cell or the foregoing composition for use in any one of L1-L3, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

L5. The foregoing cell or the foregoing composition for use in L4, wherein the neuroendocrine tumors of the lung are selected from the group consisting of pulmonary neuroendocrine cancer, large cell neuroendocrine carcinoma, and small-cell lung cancer.

L6. The foregoing cell or the foregoing composition for use in L5, wherein the tumor is small-cell lung cancer.

L7. The foregoing cell or the foregoing composition for use in any one of L1-L6, wherein the subject is a human.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1—Generation of the Presently Disclosed DLL3-Targeted CARs

The following three DLL3-targeted CARs disclosed herein were generated: J8 CAR, B2 CAR, and L22 CAR. To determine their affinity, binding domain, and specificity, antibodies including scFv of the J8 CAR, B2 CAR, and L22 CAR were analyzed. Affinity, binding domain and specificity of novel human anti-DLL3 antibodies are depicted in Table 28 below.

TABLE 28

| | Affinity | Binding | ELISA | ELISA | ELISA |
|---|---|---|---|---|---|
| Name | hDLL3 | domain | hDLL3 | hDLL1 | hDLL4 |
| B2 | 3.55 nM | EGF-like 5 | 1.205 | 0.4 | 0.043 |
| | | and EGF-like 6 | | | |
| J8 | <0.001 nM | EGF-like 5 | 1.377 | 0.095 | 0.085 |
| | | and EGF-like 6 | | | |
| L22 | 1.46 nM | EGF-like 3 | 1.639 | 0.105 | 0.056 |
| | | and EGF-like 4 | | | |

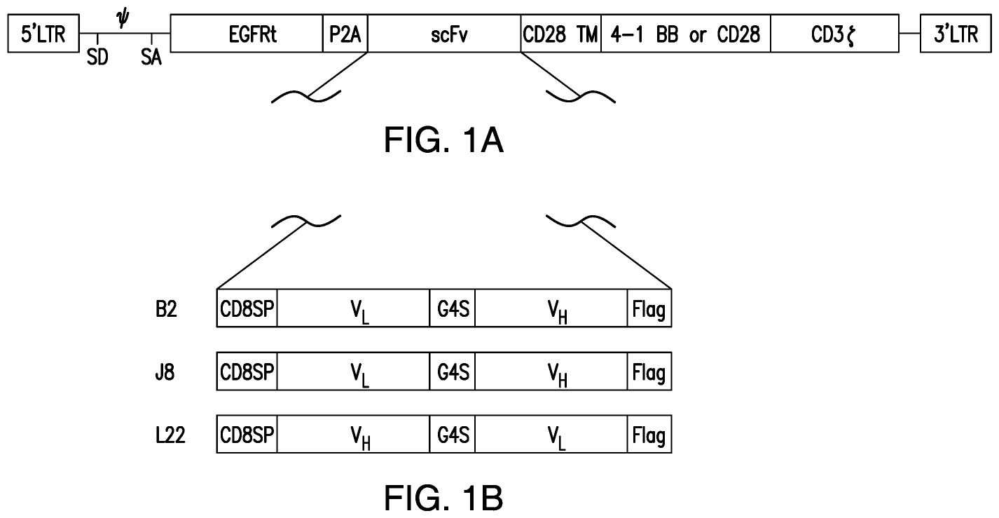

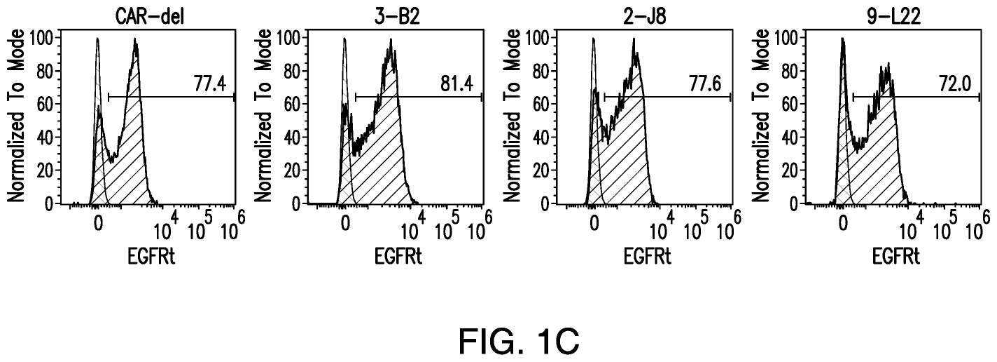

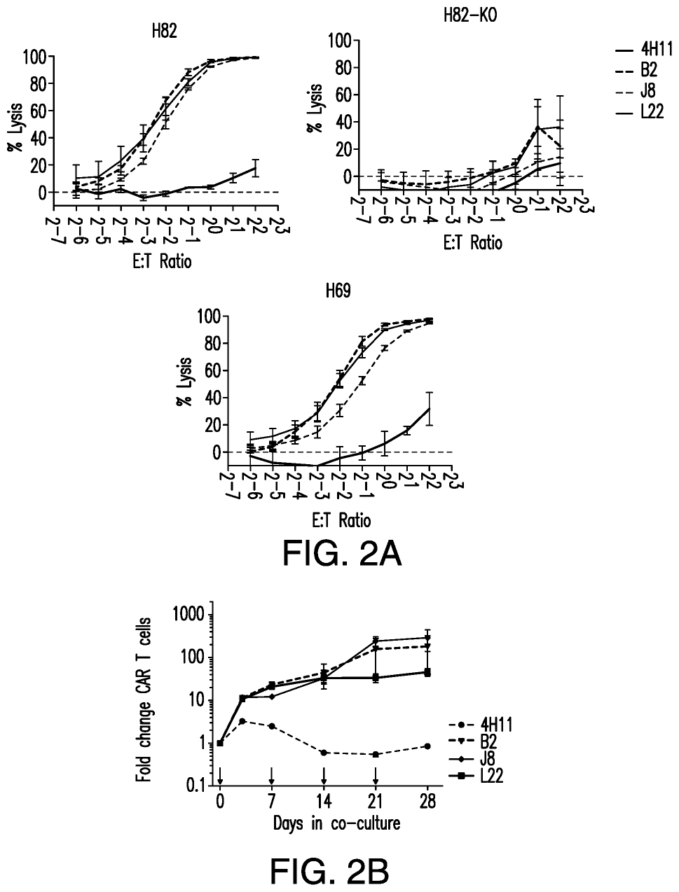

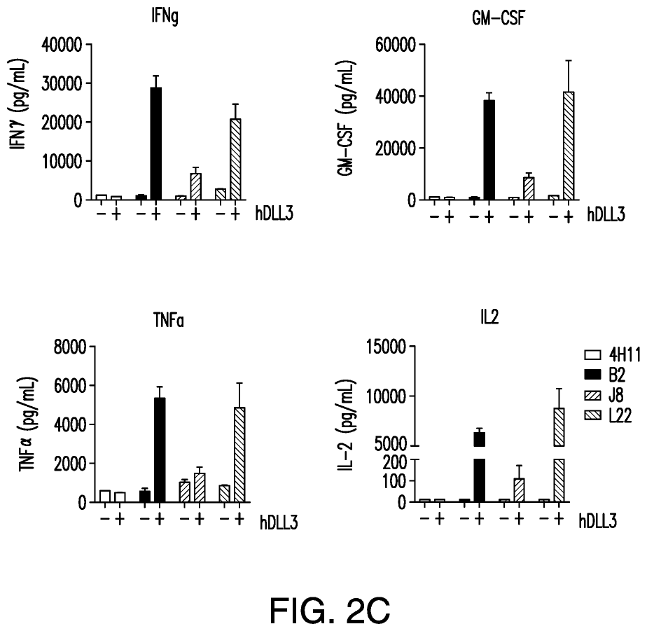

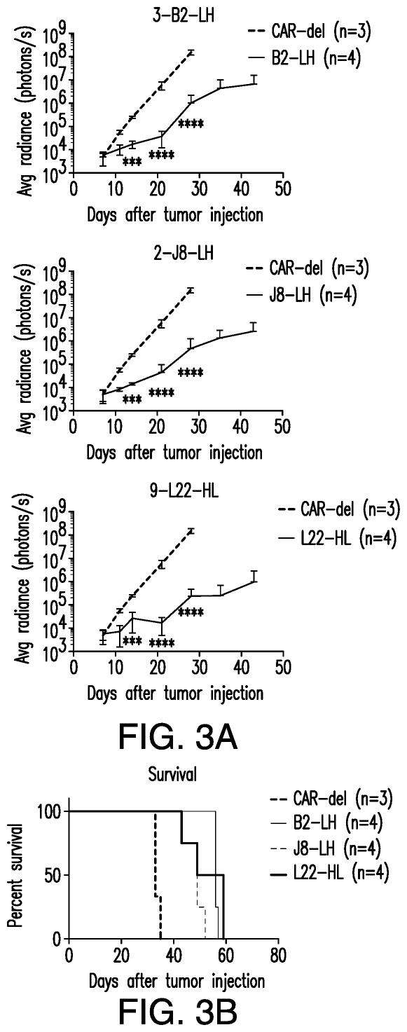

The structures of these CARs are depicted in FIGS. 1A and 1B. As shown in FIG. 1A, certain CARs include an intracellular signaling domain that comprises a CD3ζ polypeptide and a co-stimulatory signaling region comprising a 4-1BB polypeptide (e.g., an intracellular domain of human 4-1BB or a fragment thereof) (referred to as “BBz” CAR); and certain CARs include an intracellular signaling domain that comprises a CD3ζ polypeptide and a co-stimulatory signaling region comprising a CD28 polypeptide (e.g., an intracellular domain of human CD28 or a fragment thereof) (referred to as “28z” CAR). Further, as shown in FIG. 1C, these CAR are well expressed on the surface of transduced T cells. The CARs shown in following FIGS. 2A and 2B are BBz CAR, and the CARs shown in FIGS. 3A and 3B below are 28z CAR. B2 is also referred as “3-B2”. J8 is also referred to as “2-J8”. L22 is also referred to as “9-L22”.

### Example 2—Cytotoxicity of DLL3-Targeted CAR T Cells

T cells comprising a presently disclosed DLL3-targeted 28z CAR (J8, B2, and L22) were developed (FIGS. 1A-1C). The cytotoxicity and proliferation of these BBz CAR T cells were tested. As shown in FIG. 2A, human T cells comprising J8-BBzCAR, human T cells comprising B2-BBzCAR, and human T cells comprising L22-BBzCAR showed specific in vitro cytotoxicity against DLL3⁺ small cell lung cancer (SCLC) cell lines (H82 and H69) and did not show any effect against DLL3-knock out SCLC cell lines (H82-KO). Furthermore, J8-BBzCAR T cells, B2-BBzCAR T cells, and L22-BBzCAR T cells proliferated (see FIG. 2B) and secreted inflammatory cytokines (see FIG. 2C) upon stimulation with DLL3⁺ 293 cells. 4H11-CAR that does not target DLL3 was used as a negative control.

In addition, the in vivo activities of J8-28zCAR T cells, B2-28zCAR T cells, and L22-28zCAR T cells were determined. NSG mice received 3×10⁶ intravenous 28zCAR T cells 7 days after tumor cell inoculation. As shown in FIG. 3A, J8-28zCAR T cells, B2-28zCAR T cells, and L22-28zCAR T cells reduced tumor growth of metastatic H82-GFP-luciferase. Furthermore, as shown in FIG. 3B, all J8-28zCAR T cells, B2-28zCAR T cells, and L22-28zCAR T cells prolonged survival of NSG mice with systemic H82 tumors.

### Example 3—Cytotoxicity of DLL3-Targeted CAR T Cells Including Exogenous IL-18

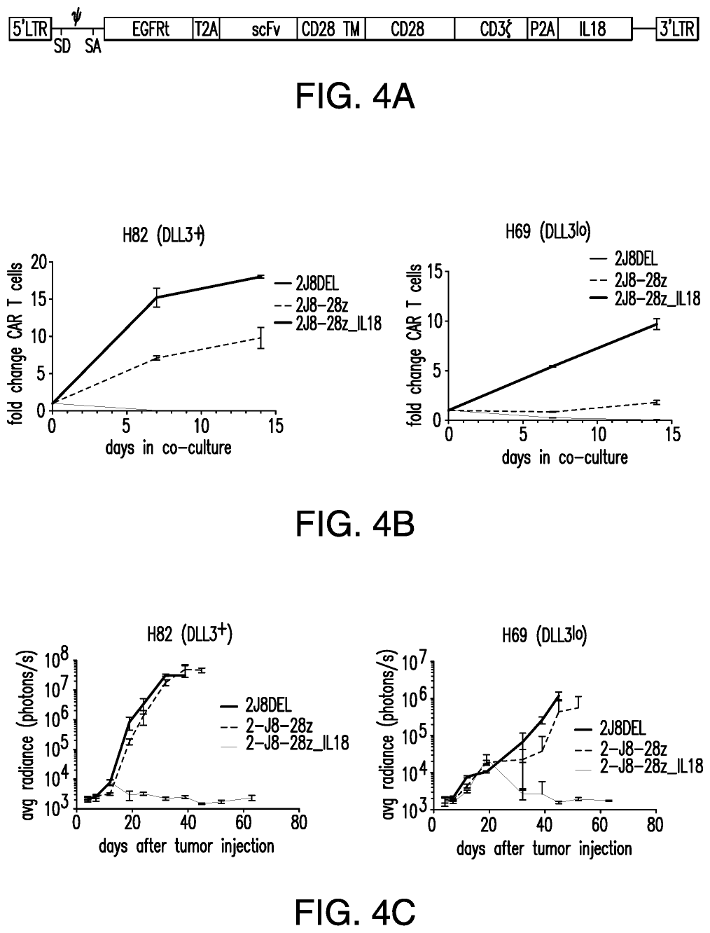

T cells comprising a presently disclosed DLL3-targeted 28z CAR (J8, also referred to as “2-J8”) and expressing an exogenous IL-18 polypeptide were developed (FIG. 4A, also referred to as “2J8-28z_IL18”). The cytotoxicity and proliferation of these 2J8-28z_IL18 CAR T cells were tested. As shown in FIG. 4B, T cells comprising 2J8-28z_IL18 proliferated (see FIG. 2B) upon stimulation with H82 tumor cells and H69 SCLC tumor cells (E:T ratio of 1:5), the latter characterized by having low expression of DLL3, compared to the controls.

In addition, the in vivo activities of 2J8-28z_IL18 T cells were determined. Mice with systemic H82 or H69 tumors received 1×10⁶ intravenous 2J8-28z_IL18 T cells 4 days (H82 tumors) or 7 days (H69 tumors) after tumor cell engraftment. As shown in FIG. 4C, 2J8-28z_IL18 showed increased antitumor efficacy. Overall, these data indicate that CAR T cell secretion of IL-18 highly increases the antitumor efficacy of the anti-DLL3 CAR T cell therapy.

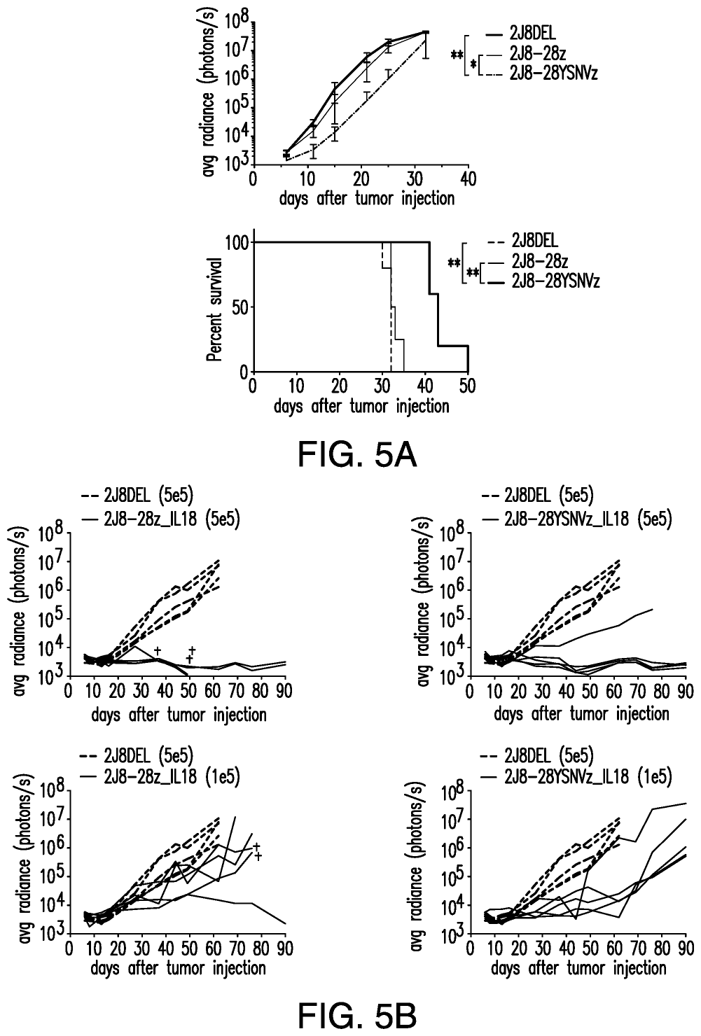

Next, it was determined whether mutations of the CD28 co-stimulatory domain could improve the efficacy of these CAR T cells. T cells comprising a presently disclosed DLL3-targeted 28z CAR (J8, also referred to as “2-J8”) were developed. As shown in FIG. 5A, the mutation of the YMNM motif in the CD28 co-stimulatory domain to YSNV (28YSNV) increased antitumor efficacy of 2J8 CAR T cells in mice with metastatic H82 tumors. Notably, the survival of mice treated with 2J8-28YSNVz CAR T cells was prolonged.

Finally, it was determined whether expression of exogenous IL-18 polypeptide could increase the anti-tumor efficacy of the CAR DLL3-targeted 28z CAR (J8, also referred to as “2J8-28YSNVz”). Thus, T cells expressing the 2J8-28YSNVz CAR and an exogenous IL-18 polypeptide (also referred to as “2J8-28YSNVz_IL18”) were developed. As seen in FIG. 5B. CAR T cells with 28YSNV co-stimulation and secreting the IL-18 cytokine (2J8-28YSNVz_IL18) demonstrated antitumor efficacy in mice with metastatic SHP-77 SCLC tumors as potent as with wildtype CD28 co-stimulation (2J8-28z_IL18). Notably, the 2J8-28YSNVz_IL18 induced less graft-versus-host-disease (GvhD).

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An antigen-recognizing receptor, comprising an extracellular antigen-binding domain, a transmembrane domain, and an intracellular signaling domain, wherein the extracellular antigen-binding domain specifically binds to DLL3, wherein the extracellular antigen-binding domain comprises:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof;
(b) a heavy chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof;
(c) a heavy chain variable region comprising a CDR1 comprising the amino acid sequence set forth in SEQ ID NO: 21 or a conservative modification thereof, CDR2 comprising the amino acid sequence set forth in SEQ ID NO: 2 or a conservative modification thereof, and a CDR3 comprising the amino acid sequence set forth in SEQ ID NO: 22 or a conservative modification thereof;
(d) a heavy chain variable region comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof;
(e) a heavy chain variable region comprising a CDR1 comprising the amino acid sequence set forth in SEQ ID NO: 21 or a conservative modification thereof, a CDR2 comprising the amino acid sequence set forth in SEQ ID NO: 38 or a conservative modification thereof, and a CDR3 comprising the amino acid sequence set forth in SEQ ID NO: 39 or a conservative modification thereof;
(f) a heavy chain variable region comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof;
(g) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 56 or a conservative modification thereof;
(h) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and CDR3 comprising the amino acid sequence set forth in SEQ ID NO: 64 or a conservative modification thereof;
(i) a heavy chain variable region comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof;
(j) a heavy chain variable region comprising a CDR1 comprising the amino acid sequence set forth in SEQ ID NO: 21 or a conservative modification thereof, a comprising the amino acid sequence set forth in SEQ ID NO: 80 or a conservative modification thereof, and a CDR3 comprising the amino acid sequence set forth in SEQ ID NO: 81 or a conservative modification thereof;
(k) a heavy chain variable region comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof;
(l) a heavy chain variable region comprising a CDR1 comprising the amino acid sequence set forth in SEQ ID NO: 96 or a conservative modification thereof, a CDR2 comprising the amino acid sequence set forth in SEQ ID NO: 97 or a conservative modification thereof, and a CDR3 comprising the amino acid sequence set forth in SEQ ID NO: 98 or a conservative modification thereof;
(m) a heavy chain variable region comprising a CDR1 comprising the amino acid sequence set forth in SEQ ID NO: 21 or a conservative modification thereof, a CDR2 comprising the amino acid sequence set forth in SEQ ID NO: 106 or a conservative modification thereof, and a CDR3 comprising the amino acid sequence set forth in SEQ ID NO: 107 or a conservative modification thereof;
(n) a heavy chain variable region comprising a CDR1 comprising the amino acid sequence set forth in SEQ ID NO: 95 or a conservative modification thereof, CDR2 comprising the amino acid sequence set forth in SEQ ID NO: 115 or a conservative modification thereof, and a CDR3 comprising the amino acid sequence set forth in SEQ ID NO: 116 or a conservative modification thereof;
(o) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 123 or a conservative modification thereof;
(p) a heavy chain variable region comprising a CDR1 comprising the amino acid sequence set forth in SEQ ID NO: 135 or a conservative modification thereof, a CDR2 comprising the amino acid sequence set forth in SEQ ID NO: 136 or a conservative modification thereof, and a CDR3 comprising the amino acid sequence set forth in SEQ ID NO: 137 or a conservative modification thereof;
(q) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 145 or a conservative modification thereof;
(r) a heavy chain variable region comprising a CDR1 comprising the amino acid sequence set forth in SEQ ID NO: 151 or a conservative modification thereof, a CDR2 comprising the amino acid sequence set forth in SEQ ID NO: 2 or a conservative modification thereof, and a CDR3 comprising the amino acid sequence set forth in SEQ ID NO: 152 or a conservative modification thereof;
(s) a heavy chain variable region comprising a CDR1 comprising the amino acid sequence set forth in SEQ ID NO: 11 or a conservative modification thereof, a CDR2 comprising the amino acid sequence set forth in SEQ ID NO: 136 or a conservative modification thereof, and a CDR3 comprising the amino acid sequence set forth in SEQ ID NO: 161 or a conservative modification thereof;
(t) a heavy chain variable region comprising a CDR1 comprising the amino acid sequence set forth in SEQ ID NO: 96 or a conservative modification thereof, CDR2 comprising the amino acid sequence set forth in SEQ ID NO: 167 or a conservative modification thereof, and a CDR3 comprising the amino acid sequence set forth in SEQ ID NO: 168 or a conservative modification thereof;
(u) a heavy chain variable region comprising a CDR1 comprising the amino acid sequence set forth in SEQ ID NO: 21 or a conservative modification thereof, a CDR2 comprising the amino acid sequence set forth in SEQ ID NO: 176 or a conservative modification thereof, and a CDR3 comprising the amino acid sequence set forth in SEQ ID NO: 177 or a conservative modification thereof;
(v) a heavy chain variable region comprising a CDR1 comprising the amino acid sequence set forth in SEQ ID NO: 184 or a conservative modification thereof, a CDR2 comprising the amino acid sequence set forth in SEQ ID NO: 185 or a conservative modification thereof, and a CDR3 comprising the amino acid sequence set forth in SEQ ID NO: 186 or a conservative modification thereof;
(w) a heavy chain variable region comprising a CDR1 comprising the amino acid sequence set forth in SEQ ID NO: 11 or a conservative modification thereof, a CDR2 comprising the amino acid sequence set forth in SEQ ID NO: 194 or a conservative modification thereof, and a CDR3 comprising the amino acid sequence set forth in SEQ ID NO: 195 or a conservative modification thereof; or
(x) a heavy chain variable region comprising a CDR1 comprising the amino acid sequence set forth in SEQ ID NO: 201 or a conservative modification thereof, a CDR2 comprising the amino acid sequence set forth in SEQ ID NO: 202 or a conservative modification thereof, and a CDR3 comprising the amino acid sequence set forth in SEQ ID NO: 203 or a conservative modification thereof.

2. The antigen-recognizing receptor of claim 1, wherein the extracellular antigen-binding domain is a single-chain variable fragment (scFv).

3. The antigen-recognizing receptor of claim 2, wherein the extracellular antigen-binding domain is a human scFv.

4. The antigen-recognizing receptor of claim 1, wherein the extracellular antigen-binding domain is a Fab, which is optionally crosslinked.

5. The antigen-recognizing receptor of claim 1, wherein the extracellular antigen-binding domain is a F(ab)₂.

6. The antigen-recognizing receptor of any one of claims 2-5, wherein one or more of the scFv, Fab and F(ab)₂ are comprised in a fusion protein with a heterologous sequence to form the extracellular antigen-binding domain.

7. The antigen-recognizing receptor of any one of claims 1-6, wherein the extracellular antigen-binding domain comprises:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof;
(b) (b) a heavy chain variable region comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; or
(c) a heavy chain variable region comprising a CDR1 comprising the amino acid sequence set forth in SEQ ID NO: 21 or a conservative modification thereof, a CDR2 comprising the amino acid sequence set forth in SEQ ID NO: 2 or a conservative modification thereof, and a CDR3 comprising the amino acid sequence set forth in SEQ ID NO: 22 or a conservative modification thereof.

8. The antigen-recognizing receptor of any one of claims 1-7, wherein the extracellular antigen-binding domain comprises:
(a) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof;
(b) a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising SEQ ID NO: 16 or a conservative modification thereof;
(c) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof;
(d) a light chain variable region comprising a CDR1 comprising the amino acid sequence set forth in SEQ ID NO: 31 or a conservative modification thereof, a CDR2 comprising SEQ ID NO: 32 or a conservative modification thereof, and a CDR3 comprising the amino acid sequence set forth in SEQ ID NO: 33 or a conservative modification thereof;
(e) a light chain variable region comprising a CDR1 comprising the amino acid sequence set forth in SEQ ID NO: 40 or a conservative modification thereof, a CDR2 comprising the amino acid sequence set forth in SEQ ID NO: 5 or a conservative modification thereof, and a CDR3 comprising the amino acid sequence set forth in SEQ ID NO: 41 or a conservative modification thereof;
(f) a light chain variable region comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof;
(g) a light chain variable region comprising a CDR1 comprising the amino acid sequence set forth in SEQ ID NO: 57 or a conservative modification thereof, a 1 CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(h) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 65 or a conservative modification thereof;
(i) a light chain variable region comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof;
(j) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 82 or a conservative modification thereof;
(k) a light chain variable region comprising a CDR1 comprising the amino acid sequence set forth in SEQ ID NO: 90 or a conservative modification thereof, a CDR2 comprising the amino acid sequence set forth in SEQ ID NO: 280 or a conservative modification thereof, and a CDR3 comprising the amino acid sequence set forth in SEQ ID NO: 91 or a conservative modification thereof;
(l) a light chain variable region comprising a CDR1 comprising the amino acid sequence set forth in SEQ ID NO: 99 or a conservative modification thereof, a CDR2 comprising the amino acid sequence set forth in SEQ ID NO: 100 or a conservative modification thereof, and a CDR3 comprising the amino acid sequence set forth in SEQ ID NO: 101 or a conservative modification thereof;
(m) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 112 or a conservative modification thereof;
(n) a light chain variable region comprising a CDR1 comprising the amino acid sequence set forth in SEQ ID NO: 117 or a conservative modification thereof, a CDR2 comprising the amino acid sequence set forth in SEQ ID NO: 100 or a conservative modification thereof, and a CDR3 comprising the amino acid sequence set forth in SEQ ID NO: 118 or a conservative modification thereof;
(o) a light chain variable region comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 125 or a conservative modification thereof;
(p) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 130 or a conservative modification thereof;
(q) a light chain variable region comprising a CDR1 comprising the amino acid sequence set forth in SEQ ID NO: 138 or a conservative modification thereof, a CDR2 comprising the amino acid sequence set forth in SEQ ID NO: 139 or a conservative modification thereof, and a CDR3 comprising the amino acid sequence set forth in SEQ ID NO: 140 or a conservative modification thereof;
(r) a light chain variable region comprising a CDR1 comprising the amino acid sequence set forth in SEQ ID NO: 57 or a conservative modification thereof, a CDR2 comprising the amino acid sequence set forth in SEQ ID NO: 146 or a conservative modification thereof, and a CDR3 comprising the amino acid sequence set forth in SEQ ID NO: 125 or a conservative modification thereof;
(s) a light chain variable region comprising a CDR1 comprising the amino acid sequence set forth in SEQ ID NO: 124 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof;
(t) a light chain variable region comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 162 or a conservative modification thereof;
(u) a light chain variable region comprising a CDR1 comprising the amino acid sequence set forth in SEQ ID NO: 169 or a conservative modification thereof, a CDR2 comprising the amino acid sequence set forth in SEQ ID NO: 170 or a conservative modification thereof, and a CDR3 comprising the amino acid sequence set forth in SEQ ID NO: 171 or a conservative modification thereof;
(v) a light chain variable region comprising a CDR1 comprising the amino acid sequence set forth in SEQ ID NO: 178 or a conservative modification thereof, a CDR2 comprising the amino acid sequence set forth in SEQ ID NO: 50 or a conservative modification thereof, and a CDR3 comprising the amino acid sequence set forth in SEQ ID NO: 179 or a conservative modification thereof;
(w) a light chain variable region comprising a CDR1 comprising the amino acid sequence set forth in SEQ ID NO: 187 or a conservative modification thereof, a CDR2 comprising the amino acid sequence set forth in SEQ ID NO: 188 or a conservative modification thereof, and a CDR3 comprising the amino acid sequence set forth in SEQ ID NO: 189 or a conservative modification thereof;
(x) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 196 or a conservative modification thereof; or
(y) a light chain variable region comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 204 or a conservative modification thereof.

9. The antigen-recognizing receptor of any one of claims 1-8, wherein the
(a) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof;
(b) a light chain variable region comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising SEQ ID NO: 16 or a conservative modification thereof;
(c) a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 23 or a conservative modification thereof.

10. The antigen-recognizing receptor of any one of claims 1-9, wherein the extracellular antigen-binding domain comprises:
(a) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
(c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23;
(d) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a heavy chain variable region comprising a CDR1 comprising the amino acid sequence set forth in SEQ ID NO: 21, a CDR2 comprising the amino acid sequence set forth in SEQ ID NO: 38, and a CDR3 comprising the amino acid sequence set forth in SEQ ID NO: 39, and a light chain variable region comprising a CDR1 comprising the amino acid sequence set forth in SEQ ID NO: 40, a CDR2 comprising the amino acid sequence set forth in SEQ ID NO: 5, and a CDR3 comprising the amino acid sequence set forth in SEQ ID NO: 41;
(f) a heavy chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48; and a light chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51;
(g) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59;
(h) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 64; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 65;
(i) a heavy chain variable region comprising a CDR1 comprising the amino acid sequence set forth in SEQ ID NO: 70, a CDR2 comprising the amino acid sequence set forth in SEQ ID NO: 71 and a CDR3 comprising the amino acid sequence set forth in SEQ ID NO: 72; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
(j) a heavy chain variable region comprising a CDR1 comprising the amino acid sequence set forth in SEQ ID NO: 21, a CDR2 comprising the amino acid sequence set forth in SEQ ID NO: 80, and a CDR3 comprising the amino acid sequence set forth in SEQ ID NO: 81; and a light chain variable region comprising a CDR1 comprising the amino acid sequence set forth in SEQ ID NO: 57, a CDR2 comprising the amino acid sequence set forth in SEQ ID NO: 58, and a region CDR3 comprising the amino acid sequence set forth in SEQ ID NO: 82;
(k) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 280, and a CDR3 comprising the amino acid sequence set forth in SEQ ID NO: 91;
(l) a heavy chain variable region comprising a CDR1 comprising the amino acid sequence set forth in SEQ ID NO: 96, a CDR2 comprising the amino acid sequence set forth in SEQ ID NO: 97, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 100, and a CDR3 comprising the amino acid sequence set forth in SEQ ID NO: 101;
(m) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(n) a heavy chain variable region comprising a CDR1 comprising the amino acid sequence set forth in SEQ ID NO: 21, a CDR2 comprising the amino acid sequence set forth in SEQ ID NO: 106, and a CDR3 comprising the amino acid sequence set forth in SEQ ID NO: 107; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 112;
(o) a heavy chain variable region comprising a CDR1 comprising the amino acid sequence set forth in SEQ ID NO: 96, a CDR2 comprising the amino acid sequence set forth in SEQ ID NO: 115, and a CDR3 comprising the amino acid sequence set forth in SEQ ID NO: 116 and a light chain variable region comprising a CDR1 comprising the amino acid sequence set forth in SEQ ID NO: 117, a CDR2 comprising the amino acid sequence set forth in SEQ ID NO: 100, and a CDR3 comprising the amino acid sequence set forth in SEQ ID NO: 118;
(p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 125;
(p) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a region CDR3 comprising the amino acid sequence set forth in SEQ ID NO: 125;
(q) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 56; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 130;
(r) a heavy chain variable region comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; and a light chain variable region comprising a CDR1 comprising the amino acid sequence set forth in SEQ ID NO: 138, a CDR2 comprising the amino acid sequence set forth in SEQ ID NO: 139, and a CDR3 comprising the amino acid sequence set forth in SEQ ID NO: 140;
(s) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 145; and a light chain variable region comprising a CDR1 comprising the amino acid sequence set forth in SEQ ID NO: 57, a CDR2 comprising the amino acid sequence set forth in SEQ ID NO: 146, and a CDR3 comprising the amino acid sequence set forth in SEQ ID NO: 125;
(t) a heavy chain variable region comprising a CDR1 comprising the amino acid sequence set forth in SEQ ID NO: 151, a CDR2 comprising the amino acid sequence set forth in SEQ ID NO: 2, and a CDR3 comprising the amino acid sequence set forth in SEQ ID NO: 152; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 82;
(u) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 123; and a light chain variable region comprising a CDR1 comprising the amino acid sequence set forth in SEQ ID NO: 124, a CDR2 comprising the amino acid sequence set forth in SEQ ID NO: 58, and a CDR3 comprising the amino acid sequence set forth in SEQ ID NO: 59;
(v) a heavy chain variable region comprising a CDR1 comprising the amino acid sequence set forth in SEQ ID NO: 11, a CDR2 comprising the amino acid sequence set forth in SEQ ID NO: 136, and a CDR3 comprising the amino acid sequence set forth in SEQ ID NO: 161; and a light chain variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 162;
(w) a heavy chain variable region comprising a CDR1 comprising the amino acid sequence set forth in SEQ ID NO: 96, a CDR2 comprising the amino acid sequence set forth in SEQ ID NO: 167, and a CDR3 comprising the amino acid sequence set forth in SEQ ID NO: 168; and a light chain variable region comprising a CDR1 comprising the amino acid sequence set forth in SEQ ID NO: 169, a CDR2 comprising the amino acid sequence set forth in SEQ ID NO: 170, and a CDR3 comprising the amino acid sequence set forth in SEQ ID NO: 171;
(x) a heavy chain variable region comprising a CDR1 comprising the amino acid sequence set forth in SEQ ID NO: 21, a CDR2 comprising the amino acid sequence set forth in SEQ ID NO: 176, and a CDR3 comprising the amino acid sequence set forth in SEQ ID NO: 177; and a light chain variable region comprising a CDR1 comprising the amino acid sequence set forth in SEQ ID NO: 178, a CDR2 comprising the amino acid sequence set forth in SEQ ID NO: 50, and a CDR3 comprising the amino acid sequence set forth in SEQ ID NO: 79;
(y) a heavy chain variable region comprising a CDR1 comprising the amino acid sequence set forth in SEQ ID NO: 184, a heavy chain variable region CDR2 comprising the amino acid sequence set forth in SEQ ID NO: 185, and a heavy chain variable region CDR3 comprising the amino acid sequence set forth in SEQ ID NO: 186; and a light chain variable region comprising a CDR1 comprising the amino acid sequence set forth in SEQ ID NO: 187, a CDR2 comprising the amino acid sequence set forth in SEQ ID NO: 188, and a CDR3 comprising the amino acid sequence set forth in SEQ ID NO: 189;
(z) a heavy chain variable region comprising a CDR1 comprising the amino acid sequence set forth in SEQ ID NO: 11, a CDR2 comprising the amino acid sequence set forth in SEQ ID NO: 194, and a CDR3 comprising the amino acid sequence set forth in SEQ ID NO: 195; and a light chain variable region comprising a CDR1 comprising the amino acid sequence set forth in SEQ ID NO: 4, a li CDR2 comprising the amino acid sequence set forth in SEQ ID NO: 5, and a CDR3 comprising the amino acid sequence set forth in SEQ ID NO: 196; or
(aa) a heavy chain variable region comprising a CDR1 comprising the amino acid sequence set forth in SEQ ID NO: 201, a CDR2 comprising the amino acid sequence set forth in SEQ ID NO: 202, and a CDR3 comprising the amino acid sequence set forth in SEQ ID NO: 203; and a light chain variable region comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 204.

11. The antigen-recognizing receptor of any one of claims 1-10, wherein the extracellular antigen-binding domain comprises:
(a) a heavy chain variable region comprising a CDR1 comprising the amino acid sequence set forth in SEQ ID NO: 1, a CDR2 comprising the amino acid sequence set forth in SEQ ID NO: 2 and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16; or
(c) a heavy chain variable region comprising a CDR1 comprising the amino acid sequence set forth in SEQ ID NO: 21, a CDR2 comprising the amino acid sequence set forth in SEQ ID NO: 2, and a CDR3 comprising the amino acid sequence set forth in SEQ ID NO: 22; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 23.

12. The antigen-recognizing receptor of any one of claims 1-11, wherein the extracellular antigen-binding domain comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

13. The antigen-recognizing receptor of any one of claims 1-12, wherein the extracellular antigen-binding domain comprises a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205.

14. The antigen-recognizing receptor of any one of claims 1-10, wherein the extracellular antigen-binding domain comprises a heavy chain variable region comprising the amino acid sequence set forth in SEQ ID NO: 7, SEQ ID NO: 17, or SEQ ID NO: 24.

15. The antigen-recognizing receptor of any one of claims 1-14, wherein the extracellular antigen-binding domain comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

16. The antigen-recognizing receptor of any one of claims 1-15, wherein the extracellular antigen-binding domain comprises a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

17. The antigen-recognizing receptor of any one of claims 1-16, wherein the extracellular antigen-binding domain comprises a light chain variable region comprising the amino acid sequence set forth in SEQ ID NO: 8, SEQ ID NO: 18, or SEQ ID NO: 25.

18. The antigen-recognizing receptor of any one of claims 1-17, wherein the extracellular antigen-binding domain comprises: (a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and (b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

19. The antigen-recognizing receptor of any one of claims 1-18, wherein the extracellular antigen-binding domain comprises: (a) a heavy chain variable region comprising the amino acid sequence set forth in SEQ ID NO: 7, SEQ ID NO: 17, SEQ ID NO: 24, SEQ ID NO: 34, SEQ ID NO: 42, SEQ ID NO: 52, SEQ ID NO: 60, SEQ ID NO: 66, SEQ ID NO: 76, SEQ ID NO: 83, SEQ ID NO: 92, SEQ ID NO: 102, SEQ ID NO: 108, SEQ ID NO: 119, SEQ ID NO: 126, SEQ ID NO: 131, SEQ ID NO: 141, SEQ ID NO: 147, SEQ ID NO: 153, SEQ ID NO: 157, SEQ ID NO: 163, SEQ ID NO: 172, SEQ ID NO: 180, SEQ ID NO: 190, SEQ ID NO: 197, or SEQ ID NO: 205; and (b) a light chain variable region comprising the amino acid sequence set forth in SEQ ID NO: 8, SEQ ID NO: 18, SEQ ID NO: 25, SEQ ID NO: 35, SEQ ID NO: 43, SEQ ID NO: 53, SEQ ID NO: 61, SEQ ID NO: 67, SEQ ID NO: 77, SEQ ID NO: 84, SEQ ID NO: 93, SEQ ID NO: 103, SEQ ID NO: 109, SEQ ID NO: 113, SEQ ID NO: 120, SEQ ID NO: 127, SEQ ID NO: 132, SEQ ID NO: 142, SEQ ID NO: 148, SEQ ID NO: 154, SEQ ID NO: 158, SEQ ID NO: 164, SEQ ID NO: 173, SEQ ID NO: 181, SEQ ID NO: 191, SEQ ID NO: 198, or SEQ ID NO: 206.

20. The antigen-recognizing receptor of any one of claims 1-19, wherein the extracellular antigen-binding domain comprises: (a) a heavy chain variable region comprising the amino acid sequence set forth in SEQ ID NO: 7, SEQ ID NO: 17, or SEQ ID NO: 24; and (b) a light chain variable region comprising the amino acid sequence set forth in SEQ ID NO: 8, SEQ ID NO: 18, or SEQ ID NO: 25.

21. The antigen-recognizing receptor of any one of claims 1-20, wherein the extracellular antigen-binding domain comprises:
(a) a heavy chain variable region comprising the amino acid sequence set forth in SEQ ID NO: 7, and a light chain variable region comprising the amino acid sequence set forth in SEQ ID NO: 8;
(b) a heavy chain variable region comprising the amino acid sequence set forth in SEQ ID NO: 17, and a light chain variable region comprising the amino acid sequence set forth in SEQ ID NO: 18;
(c) a heavy chain variable region comprising the amino acid sequence set forth in SEQ ID NO: 24, and a light chain variable region comprising the amino acid sequence set forth in SEQ ID NO: 25;
(d) a heavy chain variable region comprising the amino acid sequence set forth in SEQ ID NO: 34, and a light chain variable region comprising the amino acid sequence set forth in SEQ ID NO: 35;
(e) a heavy chain variable region comprising the amino acid sequence set forth in SEQ ID NO: 42, and a light chain variable region comprising the amino acid sequence set forth in SEQ ID NO: 43;
(f) a heavy chain variable region comprising the amino acid sequence set forth in SEQ ID NO: 52, and a light chain variable region comprising the amino acid sequence set forth in SEQ ID NO: 53;
(g) a heavy chain variable region comprising the amino acid sequence set forth in SEQ ID NO: 60, and a light chain variable region comprising the amino acid sequence set forth in SEQ ID NO: 61;
(h) a heavy chain variable region comprising the amino acid sequence set forth in SEQ ID NO: 66, and a light chain variable region comprising the amino acid sequence set forth in SEQ ID NO: 67;
(i) a heavy chain variable region comprising the amino acid sequence set forth in SEQ ID NO: 76, and a light chain variable region comprising the amino acid sequence set forth in SEQ ID NO: 77;
(j) a heavy chain variable region comprising the amino acid sequence set forth in SEQ ID NO: 83, and a light chain variable region comprising the amino acid sequence set forth in SEQ ID NO: 84;
(k) a heavy chain variable region comprising the amino acid sequence set forth in SEQ ID NO: 92, and a light chain variable region comprising the amino acid sequence set forth in SEQ ID NO: 93; and
(l) a heavy chain variable region comprising the amino acid sequence set forth in SEQ ID NO: 102, and a light chain variable region comprising the amino acid sequence set forth in SEQ ID NO: 103.
(m) a heavy chain variable region comprising the amino acid sequence set forth in SEQ ID NO: 108, and a light chain variable region comprising the amino acid sequence set forth in SEQ ID NO: 109;
(n) a heavy chain variable region comprising the amino acid sequence set forth in SEQ ID NO: 108, and a light chain variable region comprising the amino acid sequence set forth in SEQ ID NO: 113;
(o) a heavy chain variable region comprising the amino acid sequence set forth in SEQ ID NO: 119, and a light chain variable region comprising the amino acid sequence set forth in SEQ ID NO: 120;
(p) a heavy chain variable region comprising the amino acid sequence set forth in SEQ ID NO: 126, and a light chain variable region comprising the amino acid sequence set forth in SEQ ID NO: 127;
(q) a heavy chain variable region comprising the amino acid sequence set forth in SEQ ID NO: 131, and a light chain variable region comprising the amino acid sequence set forth in SEQ ID NO: 132;
(r) a heavy chain variable region comprising the amino acid sequence set forth in SEQ ID NO: 141, and a light chain variable region comprising the amino acid sequence set forth in SEQ ID NO: 142;
(s) a heavy chain variable region comprising the amino acid sequence set forth in SEQ ID NO: 147, and a light chain variable region comprising the amino acid sequence set forth in SEQ ID NO: 148;
(t) a heavy chain variable region comprising the amino acid sequence set forth in SEQ ID NO: 153, and a light chain variable region comprising the amino acid sequence set forth in SEQ ID NO: 154;
(u) a heavy chain variable region comprising the amino acid sequence set forth in SEQ ID NO: 157, and a light chain variable region comprising the amino acid sequence set forth in SEQ ID NO: 158;
(v) a heavy chain variable region comprising the amino acid sequence set forth in SEQ ID NO: 163, and a light chain variable region comprising the amino acid sequence set forth in SEQ ID NO: 164;
(w) a heavy chain variable region comprising the amino acid sequence set forth in SEQ ID NO: 172, and a light chain variable region comprising the amino acid sequence set forth in SEQ ID NO: 173;
(x) a heavy chain variable region comprising the amino acid sequence set forth in SEQ ID NO: 180, and a light chain variable region comprising the amino acid sequence set forth in SEQ ID NO: 181;
(y) a heavy chain variable region comprising the amino acid sequence set forth in SEQ ID NO: 190, and a light chain variable region comprising the amino acid sequence set forth in SEQ ID NO: 191;
(z) a heavy chain variable region comprising the amino acid sequence set forth in SEQ ID NO: 197, and a light chain variable region comprising the amino acid sequence set forth in SEQ ID NO: 198; or
(aa) a heavy chain variable region comprising the amino acid sequence set forth in SEQ ID NO: 205, and a light chain variable region comprising the amino acid sequence set forth in SEQ ID NO: 206.

22. The antigen-recognizing receptor of any one of claims 1-21, wherein the extracellular antigen-binding domain comprises:
(a) a heavy chain variable region comprising the amino acid sequence set forth in SEQ ID NO: 7, and a light chain variable region comprising the amino acid sequence set forth in SEQ ID NO: 8;
(b) a heavy chain variable region comprising the amino acid sequence set forth in SEQ ID NO: 17, and a light chain variable region comprising the amino acid sequence set forth in SEQ ID NO: 18; or
(c) a heavy chain variable region comprising the amino acid sequence set forth in SEQ ID NO: 24, and a light chain variable region comprising the amino acid sequence set forth in SEQ ID NO: 25.

23. The antigen-recognizing receptor of any one of claims 1-22, wherein the extracellular antigen-binding domain comprises a linker between a heavy chain variable region and a light chain variable region of the extracellular antigen-binding domain.

24. The antigen-recognizing receptor of claim 23, wherein the linker comprises or consists of the amino acid sequence set forth in SEQ ID NO: 209, SEQ ID NO: 210, SEQ ID NO: 211, SEQ ID NO: 212, SEQ ID NO: 213, or SEQ ID NO: 214.

25. The antigen-recognizing receptor of any one of claims 1-24, wherein the extracellular antigen-binding domain comprises a signal peptide that is covalently joined to the 5′ terminus of the extracellular antigen-binding domain.

26. The antigen-recognizing receptor of any one of claims 1-25, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

27. The antigen-recognizing receptor of any one of claims 1-26, wherein the intracellular signaling domain comprises a CD3ζ polypeptide.

28. The antigen-recognizing receptor of claim 27, wherein the CD3ζ polypeptide comprises or consists of the amino acid sequence set forth in SEQ ID NO: 221.

29. The antigen-recognizing receptor of any one of claims 1-27, wherein the intracellular signaling domain further comprises at least one co-stimulatory signaling region.

30. The antigen-recognizing receptor of claim 28, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

31. The antigen-recognizing receptor of claim 30, wherein the at least one co-stimulatory signaling region comprises a CD28 polypeptide.

32. The antigen-recognizing receptor of claim 31, wherein the CD28 polypeptide comprises or consists of amino acids 180 to 220 of SEQ ID NO: 7.

33. The antigen-recognizing receptor of claim 31, wherein the CD28 polypeptide comprises a mutated YMNM motif.

34. The antigen-recognizing receptor of claim 31 or 33, wherein the CD28 polypeptide comprises or consists of the amino acid sequence set forth in SEQ ID NO: 275, SEQ ID NO: 276, SEQ ID NO: 277, SEQ ID NO: 278, or SEQ ID NO: 279.

35. The antigen-recognizing receptor of any one of claims 1-34, wherein the antigen-recognizing receptor is a chimeric antigen receptor (CAR), or a T-cell like fusion protein.

36. The antigen-recognizing receptor of any one of claims 1-35, wherein the antigen-recognizing receptor is a CAR.

37. The antigen-recognizing receptor of any one of claims 1-36, wherein the antigen-recognizing receptor is recombinantly expressed.

38. The antigen-recognizing receptor of any one of claims 1-37, wherein the antigen-recognizing receptor is expressed from a vector.

39. The antigen-recognizing receptor of claim 38, wherein the vector is a γ-retroviral vector.

40. A cell comprising the antigen-recognizing receptor of any one of claims 1-39.

41. The cell of claim 40, wherein the cell is transduced with the antigen-recognizing receptor.

42. The cell of claim 40 or 41, wherein the antigen-recognizing receptor is constitutively expressed on the surface of the cell.

43. The cell of any one of claims 40-42, further comprising an exogenous IL-18 polypeptide.

44. The cell of claim 43, wherein the exogenous IL-18 polypeptide is a human IL-18 polypeptide.

45. The cell of any one of claims 40-44, wherein the cell is an immunoresponsive cell.

46. The cell of any one of claims 40-45, wherein the cell is a cell of the lymphoid lineage or a cell of the myeloid lineage.

47. The cell of any one of claims 40-46, wherein the cell is selected from the group consisting of a T cell, a Natural Killer (NK) cell, and a stem cell from which a lymphoid cell may be differentiated.

48. The cell of any one of claims 40-47, wherein the cell is a T cell.

49. The cell of claim 47 or 48, wherein the T cell is a cytotoxic T lymphocyte (CTL) or a regulatory T cell.

50. The cell of claim 47, wherein the stem cell is a pluripotent stem cell.

51. The cell of claim 50, wherein the pluripotent stem cell is an embryoid stem cell or an induced pluripotent stem cell.

52. A nucleic acid encoding the antigen-recognizing receptor of any one of claims 1-39.

53. A vector comprising the nucleic acid of any one of claim 52.

54. The vector of claim 53, wherein the vector is a γ-retroviral vector.

55. A host cell expressing the nucleic acid of claim 52.

56. The host cell of claim 55, wherein the host cell is a T cell.

57. A composition comprising the cell of any one of claims 40-51.

58. The composition of claim 57, which is a pharmaceutical composition further comprising a pharmaceutically acceptable carrier.

59. A lipid nanoparticle comprising the nucleic acid of claim 52.

60. A composition comprising the lipid nanoparticle of claim 59.

61. The composition of claim 60, which is a pharmaceutical composition further comprising a pharmaceutically acceptable carrier.

62. A method of treating or ameliorating a disease or disorder in a subject, comprising administering to the subject the presently disclosed cell of any one of claims 40-51, or the composition of any one of claims 57, 58, 60, or 61.

63. The method of claim 62, wherein the disease or disorder is a tumor.

64. The method of claim 63, wherein the tumor is cancer.

65. The method of any one of claims 62-64, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

66. The method of claim 65, wherein the neuroendocrine tumors of the lung are selected from the group consisting of pulmonary neuroendocrine cancer, large cell neuroendocrine carcinoma, and small-cell lung cancer.

67. The method of claim 59, wherein the tumor is small-cell lung cancer.

68. The method of any one of claims 62-67, wherein the subject is a human.

69. A kit for treating or ameliorating a disease or disorder in a subject, comprising the cell of any one of claims 40-51, the nucleic acid of claim 52, the lipid nanoparticle of claim 59, or the composition of any one of claims 57, 58, 60, or 61.

70. The kit of claim 69, wherein the kit further comprises written instructions for using the cell or composition for treating or ameliorating a disease or disorder in a subject.

71. A method for producing a DLL3-targeted antigen-recognizing receptor of any one of claims 1-39, comprising introducing into the cell a nucleic acid that encodes the antigen-recognizing receptor.

72. The cell of any one of claims 40-51 or the composition of any one of claims 57, 58, 60, or 61 for use in treating or ameliorating a disease or disorder in a subject.

73. The cell or the composition for use in claim 72, wherein the disease or disorder is a tumor.

74. The cell or the composition for use in claim 73, wherein the tumor is cancer.

75. The cell or the composition for use in any one of claims 72-74, wherein the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

76. The cell or the composition for use in claim 75, wherein the neuroendocrine tumors of the lung are selected from the group consisting of pulmonary neuroendocrine cancer, large cell neuroendocrine carcinoma, and small-cell lung cancer.

77. The cell or the composition for use in claim 76, wherein the tumor is small-cell lung cancer.

78. The cell or the composition for use in any one of claims 72-77, wherein the subject is a human.


SEQUENCE LISTING (280 sequences)

SEQ ID NO: 1 - protein, 7 aa, synthetic construct
GYTFTIY

SEQ ID NO: 2 - protein, 6 aa, synthetic construct
NPSGGS

SEQ ID NO: 3 - protein, 16 aa, synthetic construct
NDYVDYVTTDYYGMDV

SEQ ID NO: 4 - protein, 11 aa, synthetic construct
RASQGISNYLA

SEQ ID NO: 5 - protein, 7 aa, synthetic construct
AASSLQS

SEQ ID NO: 6 - protein, 9 aa, synthetic construct
QQYNSSPYT

SEQ ID NO: 7 - protein, 125 aa, synthetic construct
QVHLVQSGAEVKKPGASVKVSCKASGYTFTIYYIHWVRQAPGQGLEWMGIINPSGGSTSYAQKFQGRVTMTRDTSTSTVYMELSSLRSEDTAVYYCSGNDYVDYVTTDYYGMDVWGQGTTVTVSS

SEQ ID NO: 8 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSSPYTFGQGTKLEIK

SEQ ID NO: 9 - DNA, 375 bp, synthetic construct
  source 1..375: mol_type = other DNA
caggtgcatctggtgcagtctggggctgaggtgaagaagcctggggcctcagtgaaggtttcttgcaaggcatctggatacaccttcaccatctactatattcactgggtgcgacaggcccctggacaggggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtatattactgttcgggtaatgactacgttgactacgtaactactgactactacggtatggacgtctggggccaagggaccacggtcaccgtctcctca

SEQ ID NO: 10 - DNA, 321 bp, synthetic construct
  source 1..321: mol_type = other DNA
gacatccagatgacccagtctccatcctcactgtctgcatctgtaggagatagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcacactcaccatcagcagcctgcagcctgaagattttgcaacttattactgccaacagtataatagttccccgtacacttttggccaggggaccaagctggagatcaaa

SEQ ID NO: 11 - protein, 7 aa, synthetic construct
GFTFSSY

SEQ ID NO: 12 - protein, 6 aa, synthetic construct
SGRGGD

SEQ ID NO: 13 - protein, 8 aa, synthetic construct
ERGLGFDY

SEQ ID NO: 14 - protein, 17 aa, synthetic construct
RSSQSLLDSDDGNTYLD

SEQ ID NO: 15 - protein, 7 aa, synthetic construct
TLSYRAS

SEQ ID NO: 16 - protein, 9 aa, synthetic construct
MQHLEFPLT

SEQ ID NO: 17 - protein, 117 aa, synthetic construct
EVQLLESGGGLVQPGGSLRLSCAASGFTFSSYAMGWVRQTPGKGLEWVSTISGRGGDTYYADSVKGRFTISRDNSKNTLYLQMNSLRAEDTAIYYCAKERGLGFDYWGQGTLVTVSS

SEQ ID NO: 18 - protein, 113 aa, synthetic construct
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQHLEFPLTFGGGTKVEIK

SEQ ID NO: 19 - DNA, 351 bp, synthetic construct
  source 1..351: mol_type = other DNA
gaggtgcagctgttggagtctgggggaggcttggtacagcctggggggtccctgagactctcctgtgcagcctctggattcacctttagcagctatgccatgggctgggtccgccagactccagggaaggggctggagtgggtctcaactattagtggtcgtggtggtgacacatactacgcagactccgtgaagggccggttcaccatctccagagacaattccaagaacacgctgtatctgcaaatgaacagcctgagagccgaggacacggccatatattactgtgcgaaagagaggggactggggtttgactactggggccagggaaccctggtcaccgtctcctca

SEQ ID NO: 20 - DNA, 339 bp, synthetic construct
  source 1..339: mol_type = other DNA
gatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctgatctatacgctttcctatcgggcctctggagtcccagacaggttcagtggcagtgggtcaggcactgatttcacactgaaaatcagcagggtggaggctgaggatgttggagtttattactgcatgcaacatctagagtttccgctcactttcggcggagggaccaaggtggagatcaaa

SEQ ID NO: 21 - protein, 7 aa, synthetic construct
GYTFTSY

SEQ ID NO: 22 - protein, 15 aa, synthetic construct
GDYDILTDYYYNMDV

SEQ ID NO: 23 - protein, 9 aa, synthetic construct
LQHNSYPYT

SEQ ID NO: 24 - protein, 124 aa, synthetic construct
QVQLVQSGAEVKKPGASVKVSCKASGYTFTSYYMHWVRQAPGQGLEWMGIINPSGGSTSYAQKFRGRVTMTRDTSTSTVYMELSSLRSEDTAVYYCARGDYDILTDYYYNMDVWGQGTTVTVSS

SEQ ID NO: 25 - protein, 107 aa, synthetic construct
DIQMTQSPSAMSASVGDRVTITCRASQGISNYLAWFQQKPGKVPKRLIYAASSLQSGVPSRFSGSGSGTEFTLTISSLQPEDFATYYCLQHNSYPYTFGQGTKLEIK

SEQ ID NO: 26 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccggggcagagtcaccatgaccagggacacgtccacgagcacagtctacatggagctgagcagcctgagatctgaggacacggccgtttattactgtgcgagaggggattacgatattttgacggactactactacaatatggacgtctggggccaagggaccacggtcaccgtctcctca

SEQ ID NO: 27 - DNA, 321 bp, synthetic construct
  source 1..321: mol_type = other DNA
gacatccagatgacccagtctccatctgccatgtctgcatctgtaggagacagagtcaccatcacttgtcgggcgagtcagggcattagcaattatttagcctggtttcagcagaaaccagggaaagtccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacactttcggccaggggaccaagctggagatcaaa

SEQ ID NO: 28 - protein, 9 aa, synthetic construct
GFSLSTSGM

SEQ ID NO: 29 - protein, 5 aa, synthetic construct
DWDDN

SEQ ID NO: 30 - protein, 13 aa, synthetic construct
IRGYSGSYDAFDI

SEQ ID NO: 31 - protein, 16 aa, synthetic construct
RSSQSLLHSNGYNYLD

SEQ ID NO: 32 - protein, 7 aa, synthetic construct
LGSNRAP

SEQ ID NO: 33 - protein, 9 aa, synthetic construct
MQALQTPFT

SEQ ID NO: 34 - protein, 123 aa, synthetic construct
QVTLRESGPALVKSTQTLTLTCTFSGFSLSTSGMCVSWIRQTPGKALEWLALIDWDDNKYYSTSLKTRLIISKDTSKNQVVLTMTNMEPVDTATYYCARIRGYSGSYDAFDIWGQGTMVTVSS

SEQ ID NO: 35 - protein, 112 aa, synthetic construct
DIVMTQSPLSLPVTPGEPASISCRSSQSLLHSNGYNYLDWYLQKPGQSPHILLYLGSNRAPGVPDRFSGSGSGTDFTLKISRVEAEDVGVYYCMQALQTPFTFGPGTKVDIK

SEQ ID NO: 36 - DNA, 369 bp, synthetic construct
  source 1..369: mol_type = other DNA
caggtcaccttgagggagtctggtcctgcgctggtgaaatccacacagaccctcacactgacctgcaccttctctgggttctcactcagtactagtggaatgtgtgtgagctggatccgtcagaccccagggaaggccctggagtggcttgcactcattgattgggatgataataaatactacagcacatctctgaagaccaggctcatcatctccaaggacacctccaaaaaccaggtggtccttacaatgaccaatatggaacctgtggacacagccacgtattattgtgcacggatacgggggtatagtgggagctacgatgcttttgatatctggggccaagggacaatggtcaccgtctcttca

SEQ ID NO: 37 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacacatcctgctctatttgggttctaatcgggcccccggggtccctgacaggttcagtggcagtggatcaggcacagattttacactgaaaatcagcagagtggaggctgaggatgttggggtttattactgcatgcaagctctacaaactccattcactttcggccctgggaccaaagtggatatcaaa

SEQ ID NO: 38 - protein, 6 aa, synthetic construct
NSSGGS

SEQ ID NO: 39 - protein, 15 aa, synthetic construct
GYCTNGVYYDEDFDY

SEQ ID NO: 40 - protein, 11 aa, synthetic construct
RASQGISHYLA

SEQ ID NO: 41 - protein, 9 aa, synthetic construct
QQYNSYPLT

SEQ ID NO: 42 - protein, 124 aa, synthetic construct
QVQLVQSGAEVKKPGASVKVSCKASGYTFTSYYMHWVRQAPGQGLEWMGVINSSGGSTSYAQKFQGRVTMTRDTSTSTVYMDLSSLRSEDTAVYYCARGYCTNGVYYDEDFDYWGQGTLVTVSS

SEQ ID NO: 43 - protein, 107 aa, synthetic construct
DIQMTQSPSSLSASVGDRVTITCRASQGISHYLAWFQQKPGKAPTSLIYAASSLQSGVPSKFSGSGSGTDFTLTISSLQPEDFATYYCQQYNSYPLTFGGGTKVEIK

SEQ ID NO: 44 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggagtaatcaattctagtggtggtagcacaagctacgcacagaagttccagggcagagtcaccatgaccagggacacgtccacgagcacagtctacatggacctgagcagcctgagatctgaggacacggccgtgtattactgtgcgagagggtattgtactaatggtgtttactatgatgaggactttgactactggggccagggaaccctggtcaccgtctcctca

SEQ ID NO: 45 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagccattatttagcctggtttcagcagaaaccagggaaagcccctacgtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctctcactttcggcggagggaccaaggtggagatcaaa

SEQ ID NO: 46 - protein, 7 aa, synthetic construct
GGSISSY

SEQ ID NO: 47 - protein, 5 aa, synthetic construct
YTSGS

SEQ ID NO: 48 - protein, 10 aa, synthetic construct
EDWESWTFDI

SEQ ID NO: 49 - protein, 16 aa, synthetic construct
RSGQSLVYSDGDTYLN

SEQ ID NO: 50 - protein, 7 aa, synthetic construct
KVSNRDS

SEQ ID NO: 51 - protein, 9 aa, synthetic construct
MQGTHWPLS

SEQ ID NO: 52 - protein, 118 aa, synthetic construct
QVQLQESGPGLVKPSETLFLTCTVSGGSISSYYWTWIRQPAGKGLEWIGRIYTSGSTNYNPSLKSRVTMSVDTSKNQFSLKLSSVTAADTAVYYCAREDWESWTFDIWGQGTMVTVSS

SEQ ID NO: 53 - protein, 112 aa, synthetic construct
DVVMTQSPLSLPVTLGQPASISCRSGQSLVYSDGDTYLNWFQQRPGQSPRRLIYKVSNRDSGVPDRFSGSGSGTDFTLKISRVEAEDVGVYYCMQGTHWPLSFGGGTKVEIK

SEQ ID NO: 54 - DNA, 354 bp, synthetic construct
  source 1..354: mol_type = other DNA
caggtgcagctgcaggagtcgggcccaggactggtgaagccttcggagaccctgttcctcacctgcactgtctctggtggctccatcagtagttactactggacctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagaggactgggaatcgtggacttttgatatctggggccaagggacaatggtcaccgtctcttca

SEQ ID NO: 55 - DNA, 336 bp, synthetic construct
  source 1..336: mol_type = other DNA
gatgttgtgatgactcagtctccactctccctgcccgtcacccttggacagccggcctccatctcctgcaggtctggtcaaagcctcgtttacagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgctctctttcggcggagggaccaaggtggagatcaaa

SEQ ID NO: 56 - protein, 11 aa, synthetic construct
QLELLGNAFDI

SEQ ID NO: 57 - protein, 12 aa, synthetic construct
RASQSVSSSYLA

SEQ ID NO: 58 - protein, 7 aa, synthetic construct
GASSRAT

SEQ ID NO: 59 - protein, 9 aa, synthetic construct
QQYGSSPYT

SEQ ID NO: 60 - protein, 120 aa, synthetic construct
QVQLVQSGAEVKKPGASVKVSCKASGYTFTSYYIHWVRQAPGQGLEWMGIINPSGGSKSYAQKFQGRVTMTRDTSTGTVYMELSSLRSEDTAVYYCAGQLELLGNAFDIWGQGTMVTVSS

SEQ ID NO: 61 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 62 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatacactgggtgcgacaggcccctggacaagggcttgagtggatgggaataatcaaccctagtggtggtagtaaaagctacgcacagaagttccagggcagagtcaccatgaccagggacacgtccacgggaacagtctatatggagctgagcagcctgagatctgaggacacggccgtgtattactgtgcgggacaactggaacttttggggaatgcttttgatatctggggccaagggacaatggtcaccgtctcttca

SEQ ID NO: 63 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaagctggagatcaaa

SEQ ID NO: 64 - protein, 14 aa, synthetic construct
ERQQLDYYYFAMDV

SEQ ID NO: 65 - protein, 9 aa, synthetic construct
QQYNSYPPT

SEQ ID NO: 66 - protein, 123 aa, synthetic construct
QVQLVQSGAEVKKPGASVKVSCKASGYTFTSYYLHWVRQAPGHGLEWMAIINPSGGSTSYAQKFLGRVTMTRDTSTNTVYMELSSLRSEDTAVYYCARERQQLDYYYFAMDVWGQGTTVTVSS

SEQ ID NO: 67 - protein, 107 aa, synthetic construct
DIQMTQSPSSLSASVGDRVTITCRASQGISNYLAWFQQKPGKAPKSLIYAASSLQSGVPSKFSGSGSGTDFTLTISSLQPEDFATYYCQQYNSYPPTFGQGTKVEIK

SEQ ID NO: 68 - DNA, 369 bp, synthetic construct
  source 1..369: mol_type = other DNA
caggtgcagctggtgcagtctggggctgaggtgaagaagcctggggcctcagtgaaggtttcctgcaaggcatctggatacaccttcaccagctactatttgcactgggtgcgacaggcccctggacacgggcttgagtggatggcaataatcaaccctagtggtggtagtacaagctacgcacagaagttcctgggcagagtcaccatgaccagggacacgtccacgaacacagtctacatggagctgagcagcctgagatctgaggacacggccgtgtattactgtgcgagagaacggcagcagctggactactactacttcgctatggacgtctggggccaagggaccacggtcaccgtctcctca

SEQ ID NO: 69 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctccgacgttcggccaagggaccaaggtggaaatcaaa

SEQ ID NO: 70 - protein, 7 aa, synthetic construct
GFTFSFY

SEQ ID NO: 71 - protein, 6 aa, synthetic construct
SSSSSY

SEQ ID NO: 72 - protein, 11 aa, synthetic construct
NYDFWRGAFDI

SEQ ID NO: 73 - protein, 11 aa, synthetic construct
RASQSVSSNLA

SEQ ID NO: 74 - protein, 7 aa, synthetic construct
GASTRAT

SEQ ID NO: 75 - protein, 10 aa, synthetic construct
QQYNNWPTWT

SEQ ID NO: 76 - protein, 120 aa, synthetic construct
EVQLVESGGGLVKPGGSLRLSCAASGFTFSFYSMNWVRQAPGKGLEWVSSISSSSSYIYYADSVKGRFTISRDNAKNSLYLQMNSLRAEDTAVYYCARNYDFWRGAFDIWGQGTMVTVSS

SEQ ID NO: 77 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPTWTFGQGTKVEIK

SEQ ID NO: 78 - DNA, 360 bp, synthetic construct
  source 1..360: mol_type = other DNA
gaggtgcagctggtggagtctggggggggcctggtcaagcctggagggtccctgagactctcctgtgcagcctctggattcaccttcagtttctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaaattacgatttttggagaggtgcttttgatatctggggccaagggacaatggtcaccgtctcttca

SEQ ID NO: 79 - DNA, 324 bp, synthetic construct
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcccacgtggacgttcggccaagggaccaaggtggaaatcaaa

SEQ ID NO: 80 - protein, 6 aa, synthetic construct
NYSGGS

SEQ ID NO: 81 - protein, 11 aa, synthetic construct
QGRYSGSYLDY

SEQ ID NO: 82 - protein, 7 aa, synthetic construct
QQYGSSP

SEQ ID NO: 83 - protein, 120 aa, synthetic construct
QVQLVQSGAEVKKPGASVKVSCKASGYTFTSYYMHWVRQAPGQGLEWMGIINYSGGSKSYAQKFQGRVTMTRDTSTSTVYMELSSLRSEDTGVYYCARQGRYSGSYLDYWGQGTLVTVSS

SEQ ID NO: 84 - protein, 106 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGQGTKLEIK

SEQ ID NO: 85 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaactatagtggtggtagcaaaagctacgcacagaagttccagggcagagtcaccatgacccgggacacgtccacgagcacagtctacatggagctgagcagcctgagatctgaggacacgggcgtgtattactgtgcgagacaggggcggtatagtgggagctaccttgactactggggccagggaaccctggtcaccgtctcctca

SEQ ID NO: 86 - DNA, 318 bp, synthetic construct
  source 1..318: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttttggccaggggaccaagctggagatcaaa

SEQ ID NO: 87 - protein, 7 aa, synthetic construct
GYTFNSY

SEQ ID NO: 88 - protein, 6 aa, synthetic construct
NPSGGT

SEQ ID NO: 89 - protein, 10 aa, synthetic construct
DWGEQYGMDV

SEQ ID NO: 90 - protein, 16 aa, synthetic construct
RSSQSLLHSNGYNYLD

SEQ ID NO: 91 - protein, 9 aa, synthetic construct
MQALQTPLT

SEQ ID NO: 92 - protein, 119 aa, synthetic construct
QVQLVQSGAEVKKPGASLKVSCKTSGYTFNSYYMHWVRQAPGQGLEWMGINNPSGGTTSYAQKFQGRVTMTRDTSTSTVYMELSSLRSEDTAVYYCARDWGEQYGMDVWGQGTTVTVSS

SEQ ID NO: 93 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 94 - DNA, 357 bp, synthetic construct
  source 1..357: mol_type = other DNA
caggtgcagctggtgcagtctggggctgaggtgaagaagcctggggcctcactgaaggtttcctgcaagacatctggatacaccttcaacagctactatatgcactgggtgcgacaggcccctggacaagggcttgagtggatgggaataaacaaccctagtggtggtaccacaagctacgcacagaagttccagggcagagtcaccatgaccagggacacgtccacgagcacagtctacatggagctgagcagcctgagatctgaggacacggccgtgtattactgtgcgagagattggggggagcagtacggtatggacgtctggggccaagggaccacggtcaccgtctcctca

SEQ ID NO: 95 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccaggacagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 96 - protein, 7 aa, synthetic construct
GYTFTGY

SEQ ID NO: 97 - protein, 6 aa, synthetic construct
NPHSGA

SEQ ID NO: 98 - protein, 12 aa, synthetic construct
DSSGWYGDAFDI

SEQ ID NO: 99 - protein, 16 aa, synthetic construct
RSSQSLVHSDGNTYLS

SEQ ID NO: 100 - protein, 7 aa, synthetic construct
KISNRFS

SEQ ID NO: 101 - protein, 9 aa, synthetic construct
MQATQFPLT

SEQ ID NO: 102 - protein, 121 aa, synthetic construct
QVQLVQSGAEVKKPGASVKVSCKASGYTFTGYYMHWVRQAPGQGLEWMGWINPHSGAPNSAQKFQVRVTMTRDTSISTAYMELSRLRSDDTAVYYCARDSSGWYGDAFDIWGQGTMVTVSS

SEQ ID NO: 103 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIK

SEQ ID NO: 104 - DNA, 363 bp, synthetic construct
  source 1..363: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctcacagtggtgccccaaactctgcacagaagtttcaggtcagggtcaccatgaccagggacacgtccatcagcacagcctacatggagctgagcaggctgagatctgacgacacggccgtgtattactgtgcgagggatagcagcggctggtacggtgatgcttttgatatctggggccaagggacaatggtcaccgtctcttca

SEQ ID NO: 105 - DNA, 336 bp, synthetic construct
  source 1..336: mol_type = other DNA
gatattgtgatgacccagactccactctcctcacctgtcacccttggacagccggcctccatctcctgcaggtctagtcaaagcctcgtacacagtgatggcaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttccgctcactttcggcggagggaccaaggtggagatcaaa

SEQ ID NO: 106 - protein, 6 aa, synthetic construct
DPSGGS

SEQ ID NO: 107 - protein, 11 aa, synthetic construct
QGRYVGSYLDY

SEQ ID NO: 108 - protein, 120 aa, synthetic construct
QVQLVQSGAEVKKPGASVKVSCKASGYTFTSYYMHWVRQAPGQGLEWMGIIDPSGGSTSYAQKFQGRVTMTRDTSTNTVYMDLSSLRSEDTAVYYCARQGRYVGSYLDYWGQGTLVTVSS

SEQ ID NO: 109 - protein, 106 aa, synthetic construct; identical to SEQ ID NO: 84

SEQ ID NO: 110 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcgaccctagtggtggtagcacaagctacgcacagaagttccagggcagagtcaccatgaccagggacacgtccacgaacacagtctacatggacctgagcagcctgagatctgaggacacggccgtgtattactgtgcgagacaggggcggtatgttgggagctaccttgactactggggccagggaaccctggtcaccgtctcctca

SEQ ID NO: 111 - DNA, 318 bp, synthetic construct; identical to SEQ ID NO: 86
  source 1..318: mol_type = other DNA

SEQ ID NO: 112 - protein, 9 aa, synthetic construct
QQYNSYPYT

SEQ ID NO: 113 - protein, 107 aa, synthetic construct
DIQMTQSPSSLSASVGDRVTITCRASQGISNYLAWFQQKPGKAPKSVIYAASSLQSGVPSKFSGSGSGTDFTLTISSMQPEDFATYYCQQYNSYPYTFGQGTKLEIK

SEQ ID NO: 114 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattatttagcctggtttcagcagaaaccagggaaagcccctaagtccgtgatctatgctgcatccagtttgcaaagtggggtcccatcaaagttcagcggcagtggatctgggacagatttcactctcaccatcagcagcatgcagcctgaagattttgcaacttattactgccaacagtataatagttacccgtacacttttggccaggggaccaagctggagatcaaa

SEQ ID NO: 115 - protein, 6 aa, synthetic construct
NSYSGG

SEQ ID NO: 116 - protein, 12 aa, synthetic construct
DGSGWYGSYFDF

SEQ ID NO: 117 - protein, 16 aa, synthetic construct
RSSQSLVHSDGNTYLS

SEQ ID NO: 118 - protein, 9 aa, synthetic construct
IQTTQFPLT

SEQ ID NO: 119 - protein, 121 aa, synthetic construct
QVQLVQSGAEVRKPGASVKVSCKASGYTFTGYYMHWVRQAPGQGLEWMGWINSYSGGANYAQKFQDRVTMTRDTSVSTASMELSRLRSDDTAVYYCARDGSGWYGSYFDFWGQGTLVTVSS

SEQ ID NO: 120 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGFYYCIQTTQFPLTFGGGTKVEIK

SEQ ID NO: 121 - DNA, 363 bp, synthetic construct
  source 1..363: mol_type = other DNA
caggtgcagctggtgcagtctggggctgaggtgaggaagcctggggcctcagtgaaggtctcctgcaaggcttctggatacaccttcaccggctactatatgcactgggtgcgacaggcccctggacaagggcttgagtggatgggatggatcaactcttacagtggtggcgcaaactatgcacagaagtttcaggacagggtcaccatgaccagggacacgtccgtcagcacagcctccatggagctgagcaggctgagatctgacgacacggccgtgtattactgtgcgagagatggcagtggctggtacgggtcctactttgacttctggggccagggaaccctggtcaccgtctcctca

SEQ ID NO: 122 - DNA, 336 bp, synthetic construct
  source 1..336: mol_type = other DNA
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcgggttttattactgcatacaaactacacaatttccgctcactttcggcggagggaccaaggtggagatcaaa

SEQ ID NO: 123 - protein, 13 aa, synthetic construct
QGGAQWLVLAFDI

SEQ ID NO: 124 - protein, 11 aa, synthetic construct
RASQSVSSYLA

SEQ ID NO: 125 - protein, 9 aa, synthetic construct
QQYDSSPYT

SEQ ID NO: 126 - protein, 122 aa, synthetic construct
QVQLVQSGAEVKKPGASVQVSCKASGYTFTSYYMHWVRQAPGQGLEWMGIINPSGGSTSYAQKFQGRVTMTRDTSTSTVYMELSSLRSEDTAVYYCARQGGAQWLVLAFDIWGQGTMVTVSS

SEQ ID NO: 127 - protein, 107 aa, synthetic construct
EIVLTQSPGTLSLSPGERATLSCRASQSVSSYLAWYQQKPGQPPRLLIYGASSRATGIPDRFSGGGSGTDFTLTISRLEPEDFAVYYCQQYDSSPYTFGQGTKLEIK

SEQ ID NO: 128 - DNA, 366 bp, synthetic construct
  source 1..366: mol_type = other DNA
caggtgcagctggtgcagtctggggctgaggtgaagaagcctggggcctcagtgcaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagacaggggggagcccagtggctggtacttgcttttgatatctggggccaagggacaatggtcaccgtctcttca

SEQ ID NO: 129 - DNA, 321 bp, synthetic construct
  source 1..321: mol_type = other DNA
gaaattgtgttgacgcagtctccaggcaccctgtctttgtctccaggggaaagagccaccctctcctgcagggccagtcagagtgttagcagctacttagcctggtaccagcagaaacctggccagcctcccaggctcctcatctatggtgcatccagcagggccactggcatcccagacaggttcagtggcggtgggtctgggacagacttcactctcaccatcagcagactggagcctgaagattttgcagtgtattactgtcagcagtatgatagctcaccgtacacttttggccaggggaccaagctggagatcaaa

SEQ ID NO: 130 - protein, 9 aa, synthetic construct
QQYGNSPYT

SEQ ID NO: 131 - protein, 120 aa, synthetic construct
QVQLVQSGAEVKKPGASVKVSCKASGYTFTSYYMHWVRQAPGQGLEWMGIINPSGGSRSYAQKFQGRVTMTRDTSTSTVYMELSSLRSEDTAVYYCARQLELLGNAFDIWGQGTMVTVSS

SEQ ID NO: 132 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNSPYTFGQGTKLEIK

SEQ ID NO: 133 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagtagaagctacgcacagaagttccagggcagagtcactatgaccagggacacgtccacgagcacagtctacatggagctgagcagcctgagatctgaggacacggccgtgtattactgtgcgagacaactggaacttttggggaatgcttttgatatctggggccaagggacaatggtcaccgtctcttca

SEQ ID NO: 134 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtaactcgccgtacacttttggccaggggaccaagctggagatcaaa

SEQ ID NO: 135 - protein, 7 aa, synthetic construct
GFTFNNF

SEQ ID NO: 136 - protein, 6 aa, synthetic construct
KQDGSE

SEQ ID NO: 137 - protein, 6 aa, synthetic construct
ESYFDL

SEQ ID NO: 138 - protein, 14 aa, synthetic construct
RSSTGAVTTSNYAN

SEQ ID NO: 139 - protein, 7 aa, synthetic construct
GTNNRAP

SEQ ID NO: 140 - protein, 9 aa, synthetic construct
ALWYSNRWV

SEQ ID NO: 141 - protein, 115 aa, synthetic construct
EVQLVESGGGLVQPGGSLRLSCAASGFTFNNFWMSWVRQAPGKGLEWVANIKQDGSEKYYVDSVKGRFTISRDNAKNSLYLQMNSLRAEDTAVYYCARESYFDLWGRGTLVTVSS

SEQ ID NO: 142 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWYSNRWVFGGGTKLTVL

SEQ ID NO: 143 - DNA, 345 bp, synthetic construct
  source 1..345: mol_type = other DNA
gaggtgcagctggtggagtctgggggaggcttggtccagcctggggggtccctgagactctcctgtgcagcctctggattcacctttaataacttttggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagagagttacttcgatctctggggccgtggcaccctggtcactgtctcctca

SEQ ID NO: 144 - DNA, 327 bp, synthetic construct
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccgctgggtgttcggtggaggaaccaaactgactgtccta

SEQ ID NO: 145 - protein, 11 aa, synthetic construct
VADIVGATFDY

SEQ ID NO: 146 - protein, 7 aa, synthetic construct
GASSWAT

SEQ ID NO: 147 - protein, 120 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARVADIVGATFDYWGQGTLVTVSS

SEQ ID NO: 148 - protein, 108 aa, synthetic construct
EIVLTQSPGTLSLSPGERATLSCRASQSVSSSYLAWYQQKPGQAPRLLIYGASSWATGIPDRFSGSGSGTDFTLTISRLEPEDFAVYYCQQYDSSPYTFGQGTKLEIK

SEQ ID NO: 149 - DNA, 360 bp, synthetic construct
  source 1..360: mol_type = other DNA
caggtgcagctggtgcagtctggggctgaggtgaagaagcctggggcctcagtgaaagtttcctgcaaggcatctggatacaccttcaccagctactatatgcactgggtgcgacaggcccctggacaagggcttgagtggatgggaataatcaaccctagtggtggtagcactagctacgcacagaagttccagggcagagtcaccatgaccagggacacgtccacgagcacagtctacatggagctgagcagcctgagatctgaggacacggccgtgtattactgtgcgagagtggcggacatagtgggagctacttttgactactggggccagggaaccctggtcaccgtctcctca

SEQ ID NO: 150 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagctgggccactggcatcccagacaggttcagtggcagtgggtctgggacagacttcactctcaccatcagcagactggaacctgaagattttgcagtgtattactgtcagcagtatgatagctcaccctacacttttggccaggggaccaagctggagatcaaa

SEQ ID NO: 151 - protein, 7 aa, synthetic construct
GYTFTTY

SEQ ID NO: 152 - protein, 11 aa, synthetic construct
QGHYIGNYLDY

SEQ ID NO: 153 - protein, 120 aa, synthetic construct
QVQLVQSGAEVKKPGASVKVSCKASGYTFTTYYMHWVRQAPGQGLEWMGIINPSGGSTSYAQKFQGRVTMTRDTSTSTVYMELSSLRSEDTAVYYCARQGHYIGNYLDYWGQGTLVTVSS

SEQ ID NO: 154 - protein, 106 aa, synthetic construct; identical to SEQ ID NO: 84

SEQ ID NO: 155 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccacctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagacagggccattatattgggaactaccttgactactggggccagggaaccctggtcaccgtctcctca

SEQ ID NO: 156 - DNA, 318 bp, synthetic construct; identical to SEQ ID NO: 86
  source 1..318: mol_type = other DNA

SEQ ID NO: 157 - protein, 122 aa, synthetic construct; identical to SEQ ID NO: 126

SEQ ID NO: 158 - protein, 107 aa, synthetic construct
EIVLTQSPGTLSLSPGERATLSCRASQSVSSYLAWYQQKPGQAPRLLIYGASSRATGIPDRFSGGGSGTDFTLTISRLEPEDFAVYYCQQYGSSPYTFGQGTKLEIK

SEQ ID NO: 159 - DNA, 366 bp, synthetic construct; identical to SEQ ID NO: 128
  source 1..366: mol_type = other DNA

SEQ ID NO: 160 - DNA, 321 bp, synthetic construct
  source 1..321: mol_type = other DNA
gaaattgtgttgacgcagtctccaggcaccctgtctttgtctccaggggaaagagccaccctctcctgcagggccagtcagagtgttagcagctacttagcctggtaccagcagaaacctggccaggctcccaggctcctcatctatggtgcatccagcagggccactggcatcccagacaggttcagtggcggtgggtctgggacagacttcactctcaccatcagcagactggagcctgaagattttgcagtgtattactgtcagcagtatggtagctcaccgtacacttttggccaggggaccaagctggagatcaaa

SEQ ID NO: 161 - protein, 10 aa, synthetic construct
EWLRFGGLVY

SEQ ID NO: 162 - protein, 9 aa, synthetic construct
QQYNNWPLT

SEQ ID NO: 163 - protein, 119 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREWLRFGGLVYWGQGTLVTVSS

SEQ ID NO: 164 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 165 - DNA, 357 bp, synthetic construct
  source 1..357: mol_type = other DNA
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagagtggctacgatttgggggcttagtgtactggggccagggaaccctggtcaccgtctcctca

SEQ ID NO: 166 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggagatcaaa

SEQ ID NO: 167 - protein, 6 aa, synthetic construct
NPNSGG

SEQ ID NO: 168 - protein, 9 aa, synthetic construct
DGVITSFDY

SEQ ID NO: 169 - protein, 17 aa, synthetic construct
KSSQSVLYSSNIKTYLA

SEQ ID NO: 170 - protein, 7 aa, synthetic construct
WASTRES

SEQ ID NO: 171 - protein, 9 aa, synthetic construct
QQYYSAPYT

SEQ ID NO: 172 - protein, 118 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDGVITSFDYWGQGTLVTVSS

SEQ ID NO: 173 - protein, 113 aa, synthetic construct
DIVMTQSPDSLAVSLGERATINCKSSQSVLYSSNIKTYLAWYQQKPGQPPKLLIYWASTRESGVPDRFSGGGSGTDFTLTITSLQAEDVAVYYCQQYYSAPYTFGQGTKLEIK

SEQ ID NO: 174 - DNA, 354 bp, synthetic construct
  source 1..354: mol_type = other DNA
caggtgcagctggtgcagtctggggctgaggtgaagaagcctggggcctcagtgaaggtctcctgcaaggcttctggatacactttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagacggggtgattacgtcttttgactactggggccagggaaccctggtcaccgtctcctca

SEQ ID NO: 175 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacattaagacctacttagcttggtaccagcagaaaccaggacagcctcctaagctgctcatttactgggcatctacccgggaatccggagtccctgaccgattcagtggcggcgggtctgggacagatttcactctcaccatcaccagcctgcaggctgaagatgtggcagtttattactgtcagcagtattatagtgctccgtacacttttggccaggggaccaagctggagatcaaa

SEQ ID NO: 176 - protein, 6 aa, synthetic construct
DPSDGS

SEQ ID NO: 177 - protein, 11 aa, synthetic construct
DREYNYYGLDV

SEQ ID NO: 178 - protein, 16 aa, synthetic construct
RSSQSLVYRDGNTYLN

SEQ ID NO: 179 - protein, 9 aa, synthetic construct
MQGTHWPPT

SEQ ID NO: 180 - protein, 120 aa, synthetic construct
QVQLVQSGAEVKKPGASVKVSCKASGYTFTSYYIHWVRQAPGQGLEWMGIIDPSDGSTNYAQKFQGRVTMTRDTSTSTVYMELSSLRSEDTAVYYCARDREYNYYGLDVWGQGTTVTVSS

SEQ ID NO: 181 - protein, 112 aa, synthetic construct
DVVMTQSPLSLPVTLGQPASISCRSSQSLVYRDGNTYLNWFQQRPGQSPRRLIYKVSNRDSGVPDRFRGSGSGTDFTLKISRVEAEDVGVYYCMQGTHWPPTFGQGTKVEIK

SEQ ID NO: 182 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatacactgggtgcgacaggcccctggacaagggcttgagtggatgggaataatcgacccaagtgatggtagcacaaactacgcacagaagttccagggcagagtcaccatgaccagggacacgtccacgagcacagtctacatggagctgagcagcctgagatctgaggacacggccgtgtattactgtgcgagagatcgggaatataactactacggtttggacgtctggggccaagggaccacggtcaccgtctcctca

SEQ ID NO: 183 - DNA, 336 bp, synthetic construct
  source 1..336: mol_type = other DNA
gatgttgtgatgactcagtctccactctccctgcccgtcacccttggacagccggcctccatctcctgcaggtctagtcaaagcctcgtataccgtgatggaaacacctacttgaattggtttcagcagaggccaggccaatctccaaggcgcctaatttataaggtttctaaccgggactctggggtcccagacagattccgcggcagtgggtcaggcactgatttcacactgaaaatcagccgggtggaggctgaggatgttggggtttattactgcatgcaaggtacacactggcctccgacgttcggccaagggaccaaggtggaaatcaaa

SEQ ID NO: 184 - protein, 8 aa, synthetic construct
GFTFSNTW

SEQ ID NO: 185 - protein, 10 aa, synthetic construct
IKSKSDGGTT

SEQ ID NO: 186 - protein, 10 aa, synthetic construct
TQYYWNSFDY

SEQ ID NO: 187 - protein, 11 aa, synthetic construct
QASQDISNYLN

SEQ ID NO: 188 - protein, 7 aa, synthetic construct
DASNLET

SEQ ID NO: 189 - protein, 9 aa, synthetic construct
QQYDNLPLT

SEQ ID NO: 190 - protein, 119 aa, synthetic construct
EVQLVESGGGLVKPGGSLRLSCAASGFTFSNTWMSWVRQAPGKGLEWVGRIKSKSDGGTTDYAAPVKGRFTISRDDSKNTLYLQMNSLKTEDTAVYYCTQYYWNSFDYWGQGTLVTVSS

SEQ ID NO: 191 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 192 - DNA, 357 bp, synthetic construct
  source 1..357: mol_type = other DNA
gaggtgcagctggtggagtctggggggggcttggtaaagcctggggggtcccttagactctcctgtgcagcctctggattcactttcagtaacacctggatgagctgggtccgccaggctccagggaaggggctggagtgggttggccgtattaaaagcaaatctgatggtgggacaacagactacgctgcacccgtgaaaggcagattcaccatctcaagagatgattcaaaaaacacgctgtatctgcaaatgaacagcctgaaaaccgaggacacagccgtgtattactgtacccagtattattggaactcctttgactactggggccagggaaccctggtcaccgtctcctca

SEQ ID NO: 193 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgctcactttcggcggagggaccaaggtggagatcaaa

SEQ ID NO: 194 - protein, 6 aa, synthetic construct
KEDGSE

SEQ ID NO: 195 - protein, 9 aa, synthetic construct
DPGWAPFDY

SEQ ID NO: 196 - protein, 9 aa, synthetic construct
QQYNSFPYT

SEQ ID NO: 197 - protein, 118 aa, synthetic construct
EVQLVESGGGLVQPGGSQRLSCAASGFTFSSYWMNWVRQAPGKGLEWVANIKEDGSEKYYVDSVKGRFTISRDNAKNSLYLQMNSLRAEDTAVYYCARDPGWAPFDYWGQGTLVTVSS

SEQ ID NO: 198 - protein, 107 aa, synthetic construct
DIQMSQSPSSLSASVGDRVTITCRASQGISNYLAWFQQKPGKAPKSLIYAASSLQSGVPSKFSGSGSGTDFTLAISSLQPEDFATYYCQQYNSFPYTFGQGTTLEIK

SEQ ID NO: 199 - DNA, 354 bp, synthetic construct
  source 1..354: mol_type = other DNA
gaggtgcagctggtggagtctgggggaggcttggtccagcctggggggtcccagagactctcctgtgcagcctctggattcacctttagtagctattggatgaactgggtccgccaggctccagggaaggggctggagtgggtggccaacataaaggaagatggaagtgagaaatactatgtggactctgtgaagggccgattcaccatctccagagacaacgccaagaactcactgtatctgcaaatgaacagcctgagagccgaggacacggctgtgtattactgtgcgagagatccgggctgggctccctttgactactggggccagggaaccctggtcaccgtctcctca

SEQ ID NO: 200 - DNA, 321 bp, synthetic construct
  source 1..321: mol_type = other DNA
gacatccagatgtcccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcgccatcagcagcctgcagcctgaagattttgcaacttattactgccaacagtataatagtttcccgtacacttttggccaggggaccacgctggagatcaaa

SEQ ID NO: 201 - protein, 7 aa, synthetic construct
GGSINSY

SEQ ID NO: 202 - protein, 5 aa, synthetic construct
FYSGI

SEQ ID NO: 203 - protein, 10 aa, synthetic construct
IGVAGFYFDY

SEQ ID NO: 204 - protein, 9 aa, synthetic construct
QQYGTSPLT

SEQ ID NO: 205 - protein, 118 aa, synthetic construct
QVQLQESGPGLVKPSETLSLTCTVSGGSINSYYWSWIRQPPGKGLEWIGYIFYSGITNYNPSLKSRVTISLDTSKNQFSLKLSSVTAADTAVYYCARIGVAGFYFDYWGQGTLVTVSS

SEQ ID NO: 206 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGTSPLTFGGGTKVEIK

SEQ ID NO: 207 - DNA, 354 bp, synthetic construct
  source 1..354: mol_type = other DNA
caggtgcagctgcaggagtcgggcccaggactggtgaagccttcggagaccctgtccctcacctgcactgtctctggtggctccatcaatagttactactggagctggatccggcagcccccagggaagggactggagtggattgggtatatcttttacagtgggatcaccaactacaacccctccctcaagagtcgagtcaccatatcattagacacgtccaagaaccagttctccctgaagctgagctctgtgaccgctgcggacacggccgtgtattactgtgcgagaatcggcgtggctggtttttactttgactactggggccagggaaccctggtcaccgtctcctca

SEQ ID NO: 208 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccgctcactttcggcggagggaccaaggtggagatcaaa

SEQ ID NO: 209 - protein, 19 aa, synthetic construct
GGGGSGGGGSGGGSGGGGS

SEQ ID NO: 210 - protein, 15 aa, synthetic construct
GGGGSGGGGSGGGGS

SEQ ID NO: 211 - protein, 24 aa, synthetic construct
GGGGSGGGGSGGGGSGGGSGGGGS

SEQ ID NO: 212 - protein, 29 aa, synthetic construct
GGGGSGGGGSGGGGSGGGGSGGGSGGGGS

SEQ ID NO: 213 - protein, 5 aa, synthetic construct
GGGGS

SEQ ID NO: 214 - protein, 10 aa, synthetic construct
GGGGSGGGGS

SEQ ID NO: 215 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 216 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 217 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 218 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 219 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 220 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 221 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 222 - protein, 336 aa, synthetic construct
AGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGC

SEQ ID NO: 223 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 224 - protein, 4 aa, synthetic construct
YMNM

SEQ ID NO: 225-226 - skipped (2 entries)

SEQ ID NO: 227 - protein, 4 aa, synthetic construct
YENV

SEQ ID NO: 228 - protein, 4 aa, synthetic construct
YSNV

SEQ ID NO: 229 - protein, 4 aa, synthetic construct
YKNL

SEQ ID NO: 230 - protein, 4 aa, synthetic construct
YENQ

SEQ ID NO: 231 - protein, 4 aa, synthetic construct
YKNI

SEQ ID NO: 232 - protein, 4 aa, synthetic construct
YINQ

SEQ ID NO: 233 - protein, 4 aa, synthetic construct
YHNK

SEQ ID NO: 234 - protein, 4 aa, synthetic construct
YVNQ

SEQ ID NO: 235 - protein, 4 aa, synthetic construct
YLNP

SEQ ID NO: 236 - protein, 4 aa, synthetic construct
YLNT

SEQ ID NO: 237 - protein, 4 aa, synthetic construct
YDND

SEQ ID NO: 238 - protein, 4 aa, synthetic construct
YENI

SEQ ID NO: 239 - protein, 4 aa, synthetic construct
YENL

SEQ ID NO: 240 - protein, 4 aa, synthetic construct
YKNQ

SEQ ID NO: 241 - protein, 4 aa, synthetic construct
YKNV

SEQ ID NO: 242 - protein, 4 aa, synthetic construct
YANG

SEQ ID NO: 243 - protein, 4 aa, synthetic construct
YMDM

SEQ ID NO: 244 - protein, 4 aa, synthetic construct
YMPM

SEQ ID NO: 245 - protein, 4 aa, synthetic construct
YMRM

SEQ ID NO: 246 - protein, 4 aa, synthetic construct
YMSM

SEQ ID NO: 247 - skipped

SEQ ID NO: 248 - protein, 4 aa, synthetic construct
YTHM

SEQ ID NO: 249 - protein, 4 aa, synthetic construct
YVLM

SEQ ID NO: 250 - protein, 4 aa, synthetic construct
YIAM

SEQ ID NO: 251 - protein, 4 aa, synthetic construct
YVEM

SEQ ID NO: 252 - protein, 4 aa, synthetic construct
YVKM

SEQ ID NO: 253 - protein, 4 aa, synthetic construct
YVPM

SEQ ID NO: 254 - skipped

SEQ ID NO: 255 - protein, 4 aa, synthetic construct
YMAP

SEQ ID NO: 256-257 - skipped (2 entries)

SEQ ID NO: 258 - protein, 4 aa, synthetic construct
YMNV

SEQ ID NO: 259 - protein, 4 aa, synthetic construct
YENM

SEQ ID NO: 260 - protein, 4 aa, synthetic construct
YMNQ

SEQ ID NO: 261 - protein, 4 aa, synthetic construct
YMNL

SEQ ID NO: 262 - protein, 4 aa, synthetic construct
YSNM

SEQ ID NO: 263 - skipped

SEQ ID NO: 264 - protein, 4 aa, synthetic construct
YGGG

SEQ ID NO: 265 - protein, 4 aa, synthetic construct
YAAA

SEQ ID NO: 266 - protein, 4 aa, synthetic construct
YFFF

SEQ ID NO: 267 - protein, 4 aa, synthetic construct
YETV

SEQ ID NO: 268 - protein, 4 aa, synthetic construct
YQQQ

SEQ ID NO: 269 - protein, 4 aa, synthetic construct
YHAE

SEQ ID NO: 270 - protein, 4 aa, synthetic construct
YLDL

SEQ ID NO: 271 - protein, 4 aa, synthetic construct
YLIP

SEQ ID NO: 272 - protein, 4 aa, synthetic construct
YLRV

SEQ ID NO: 273 - protein, 4 aa, synthetic construct
YTAV

SEQ ID NO: 274 - protein, 4 aa, synthetic construct
YVHV

SEQ ID NO: 275 - protein, 41 aa, synthetic construct
RSKRSRLLHSDYENVTPRRPGPTRKHYQPYAPPRDFAAYRS

SEQ ID NO: 276 - protein, 41 aa, synthetic construct
RSKRSRLLHSDYKNITPRRPGPTRKHYQPYAPPRDFAAYRS

SEQ ID NO: 277 - protein, 41 aa, synthetic construct
RSKRSRLLHSDYMDMTPRRPGPTRKHYQPYAPPRDFAAYRS

SEQ ID NO: 278 - protein, 41 aa, synthetic construct
RSKRSRLLHSDYGGGTPRRPGPTRKHYQPYAPPRDFAAYRS

SEQ ID NO: 279 - protein, 41 aa, synthetic construct
RSKRSRLLHSDYSNVTPRRPGPTRKHYQPYAPPRDFAAYRS

SEQ ID NO: 280 - protein, 7 aa, synthetic construct
LGSNRAS